(19) United States
(12) Patent Application Publication
(10) Pub. No.: US 2018/0008671 A1
(43) Pub. Date: Jan. 11, 2018

(54) PROLONGED ANTI-DIABETIC EFFECT OF FIBROBLAST GROWTH FACTOR 1 (FGF1)

(71) Applicant: UNIVERSITY OF WASHINGTON, Seattle, WA (US)

(72) Inventors: Michael W. SCHWARTZ, Seattle, WA (US); Jarrad SCARLETT, Seattle, WA (US)

(73) Assignee: UNIVERSITY OF WASHINGTON, Seattle, WA (US)

(21) Appl. No.: 15/546,895

(22) PCT Filed: Feb. 10, 2016
(86) PCT No.: PCT/US16/17358

§ 371 Date: Jul. 27, 2017

Related U.S. Application Data
(60) Provisional application No. 62/114,451, filed on Feb. 10, 2015.
(60) Provisional application No. 62/217,344, filed on Sep. 11, 2015.

(51) Int. Cl. A61K 38/18; A61K 31/727; A61K 31/4439; A61K 45/06; A61K 8/14

(52) U.S. Cl. CPC A61K 38/1825; A61K 45/06; A61K 8/14; A61K 31/4439; A61K 31/727

(57) ABSTRACT

Disclosed are compositions and methods for inducing sustained diabetes remission by single administration of FGF1 to the brain. The composition and methods described herein result in basal glucose clearance by using a dosage of FGF1 that is lower than that needed for systemic efficacy and is devoid of the risk of hypoglycemia and changes in body weight, food intake, hepatic glucose production, insulin secretion or insulin sensitivity.

### CROSS-REFERENCE TO RELATED APPLICATION

This application claims benefit under 35 U.S.C. §119(e) of U.S. Provisional Application Nos. 62/114,451, filed Feb. 10, 2015 and 62/217,344, filed Sep. 11, 2015, the contents of which are incorporated herein by reference in their entireties.

### FUNDING SUPPORT

This invention was made with government support under Grant No. 1 R01 DK101997-01, awarded by the National Institutes of Health (NIH). The government has certain rights in the invention.

### TECHNICAL FIELD

The present invention relates to compositions and methods for the treatment of diabetes.

### SEQUENCE LISTING

The instant application contains a Sequence Listing which has been submitted electronically in ASCII format and is hereby incorporated by reference in its entirety. Said ASCII copy, created on Feb. 5, 2016, is named 034186-086312-PCT_SL.txt and is 261,550 bytes in size.

### BACKGROUND

Type 2 diabetes (T2D) is among the most common and costly disorders worldwide (1). Current medical therapy for T2D combines daily administration of one or more drugs that transiently lower blood glucose (BG) levels with frequent BG monitoring to optimize glycemic control and avert hypoglycemia. Thiazolidinediones (TZD) and metformin are oral anti-diabetic drugs that, when administered alone or in combination have a glucose lowering effects in patients with T2D and reduce plasma insulin concentrations. These currently available drugs work by increasing insulin sensitivity, whereas insulin therapy raises plasma insulin levels, and each carries the risk of causing glucose levels to drop below the normal range, potentially, resulting in life-threatening hypoglycemia. These drugs can, also have side effects including weight gain, nausea, and fatigue, as well as more serious cardiovascular and liver complications. Much of the effort, cost and risk associated with these established approaches to diabetes treatment would be mitigated by medical strategies for inducing diabetes remission. While this goal can be achieved in patients undergoing bariatric surgery (2,3), such procedures are not a viable solution to the public health crisis posed by T2D, and a medical strategy for realizing this outcome has yet to be identified.

Historically, diabetes drug development has focused primarily on pancreatic islet β cells and insulin-sensitive tissues as targets, based in part on the contribution made by defective insulin secretion or action (or both) to the development of impaired glucose tolerance (IGT) and T2D. However, an increasing body of evidence implicates the brain both in the control of glucose homeostasis and as a target for T2D treatment (4-6). In rodent models of T2D, hyperglycemia can be ameliorated transiently by either systemic or intracerebroventricular (icv) administration of fibroblast growth factor FGF19 (7-10) or FGF21 (11,12). FGF1, another member of the FGF family, is implicated in diverse processes ranging from brain development to wound healing, angiogenesis, inflammation and adipocyte differentiation (13). Interestingly, in the brain, FGF1 is synthesized by neurons, astrocytes, and ependymal cells (14,15) and central FGF1 administration can enhance learning and memory (15), reduce food intake (16), and limit damage associated with ischemic stroke or neurodegenerative disease (17,18). Mice lacking FGF1 develop insulin resistance and diabetes when challenged with a high fat diet, implying a physiological role for FGF1 in glucose homeostasis (19). While the other members of FGF family including FGF19 and FGF21, exert their biological role by binding to and activating a limited subset of FGF receptors (FGFR) via an interaction that requires the co-receptor β-Klotho, the tissue growth factor FGF1, is able to bind and activate all known FGFR isoforms without the need for β-Klotho (20). Systemic administration of FGF1 elicits transient glucose lowering lasting up to 42 h (21) which is longer than the effect elicited by either FGF19 (7) or FGF21 (22).

The steadily increasing prevalence of this disease, combined with the inability of most patients to achieve recommended glycemic targets (23), creates a compelling need for new treatment options. Accordingly, there is an unmet need for a therapeutic option that achieves sustained diabetic remission and yet do not cause hypoglycemia.

### SUMMARY

Described herein are compositions and methods to treat metabolic disorders involving abnormally elevated blood glucose levels by administration of FGF1 polypeptide to the brain. The inventors have shown that in rodent models of T2D, as little as one administration of FGF1 to the brain normalizes blood glucose levels and induces prolonged diabetes remission. This outcome is not observed following systemic FGF1 administration. Moreover, the prolonged diabetes remission induced by administration of FGF1 to the brain can be induced by a dose that is 10-fold lower than that required to achieve transient blood glucose reduction via systemic administration. Thus, the anti-diabetic effect of centrally administered FGF1 is not mediated by leakage from the brain to peripheral tissues. Furthermore, as opposed to anti-diabetic effect by systemic administration of FGF1, the anti-diabetic effect of FGF1 administration into the brain is independent of significant changes of insulin sensitivity, basal insulin levels or glucose-induced insulin secretion. Unlike current diabetes treatment strategies, FGF1-based therapeutic administration to the brain does not induce either hypoglycemia even in normal, non-diabetic animals or lasting changes of body weight or food intake.

Described herein are compositions and methods of inducing a prolonged blood glucose-normalizing effect involving administering FGF1 polypeptide to the brain, at a lower effective dosage than that required for transient blood glucose lowering when administered systemically.

Thus in one aspect, described herein are pharmaceutical compositions comprising a unit dose of a Fibroblast Growth Factor 1 (FGF1) polypeptide preparation comprising a pharmaceutically acceptable carrier and formulated for administration to the brain.

In one embodiment, the composition is formulated for administration via an intracerebroventricular, intranasal, intracranial, intracelial, intracerebellar, or intrathecal administration route.

In some embodiments, the composition is formulated for administration via an intranasal route and further comprises a ganglioside and/or a phosphotidylserine.

In some embodiments, the composition is formulated for administration via an intranasal route and further comprises saccharides selected from the group of cyclodextrins, disaccharides, polysaccharides, and combinations thereof.

In some embodiments of the aspects noted above, the pharmaceutical composition further comprises another FGF family member polypeptide.

In some embodiments, the FGF1 polypeptide is a human FGF1 polypeptide.

In some embodiments, the FGF1 polypeptide has at least 95% amino acid sequence identity to SEQ ID NO:1 and retains at least 80% of the biological activity of human FGF1 of SEQ ID NO: 1.

In some embodiments, the FGF1 polypeptide is a human recombinant polypeptide.

In some embodiments, the FGF1 polypeptide comprises amino acids 1-155 of SEQ ID NO: 1.

In some embodiments, the FGF1 polypeptide comprises at least amino acids 25-155 of SEQ ID NO: 1.

In some embodiments, the pharmaceutical composition is contained in a delivery device selected from the group consisting of a syringe, a blunt tip syringe, a catheter, an inhaler, a nebulizer, a nasal spray pump, a nasal irrigation pump or nasal lavage pump, and an implantable pump.

In some embodiments, the FGF1 polypeptide is formulated with a lipophilic molecular group.

In some embodiments, the FGF1 polypeptide is encapsulated in a liposome or a nanoparticle.

In some embodiments, FGF1 polypeptide is fused to a carrier polypeptide.

In some embodiments, the dose of Fibroblast Growth Factor 1 (FGF1) polypeptide is less than 50% of the unit dose required to treat diabetes via systemic administration.

In some embodiments, the unit dose comprises less than about 100 μg of the FGF1 polypeptide.

In one aspect, the technology described herein relates to a pharmaceutical composition comprising a unit dose of a Fibroblast Growth Factor 1 (FGF1) polypeptide preparation comprising a pharmaceutically acceptable carrier and formulated for administration to the brain, wherein the unit dose of FGF1 polypeptide is 100 μg or less.

In another aspect, the technology described herein relates to a pharmaceutical composition comprising a unit dose of a Fibroblast Growth Factor 1 (FGF1) polypeptide preparation comprising a pharmaceutically acceptable carrier and formulated for administration to the brain, wherein the unit dose of FGF1 polypeptide is less than half of the unit dose required to transiently normalize blood glucose levels when the FGF1 polypeptide is administered systemically.

In one aspect, the technology described herein relates to a pharmaceutical composition formulated for administering an FGF1 polypeptide to the brain, the composition comprising an FGF1 polypeptide and heparin.

In one aspect, the technology described herein relates to a pharmaceutical composition formulated for administering an FGF1 polypeptide to the brain, the composition comprising an FGF1 polypeptide and heparan sulfate.

In another aspect, the technology described herein relates to a method of treating a metabolic disorder in a subject, the method comprising administering a unit dose of a pharmaceutical composition comprising an FGF1 polypeptide preparation as described herein to the brain of a subject having a metabolic disorder, wherein the metabolic disorder is treated.

In some embodiments, the administration is intracerebroventricular administration, intranasal administration, intracranial administration, intracerebellar administration, intracelial administration, or intrathecal administration.

In some embodiments, the metabolic disorder is a disorder characterized by or involving abnormally elevated blood glucose levels.

In some embodiments, the metabolic disorder is selected from the group consisting of type 2 diabetes, gestational diabetes, drug-induced diabetes, high blood glucose, insulin resistance and metabolic syndrome.

In some embodiments, the method further comprises the step, prior to the administering step, of diagnosing the patient as having a metabolic disorder.

In some embodiments, prior to administration of the pharmaceutical composition the subject has a blood glucose level above the normal range, and wherein administration of the composition lowers blood glucose level to within the normal range.

In some embodiments, the administration of the pharmaceutical composition does not result in hypoglycemia.

In some embodiments, the administration does not result in a sustained loss of body weight and/or reduced food intake.

In some embodiments of the above noted aspects, the unit dose of the pharmaceutical composition required to normalize blood glucose level is less than 50% of the unit dose required to transiently normalize blood glucose when an FGF1 polypeptide is administered systemically.

In some embodiments of the above noted aspects the unit dose administered comprises 100 μg or less of the FGF1 polypeptide.

In some embodiments, a single unit dose of the administered pharmaceutical composition normalizes blood glucose level in the subject for at least one week.

Given the prolonged effects, re-administration can be performed, if necessary, when blood glucose normalization diminishes as evidenced by periodic blood glucose level monitoring. While longer intervals for FGF1 polypeptide administration to the brain can be achieved, if necessary (e.g., if fasting blood glucose levels rise outside of the normal range), re-administration can be performed weekly, biweekly, monthly, bimonthly, every three months, every 4 months, every 5 months, every 6 months or more.

In some embodiments, the method of any one of the foregoing aspects further comprises administering another FGF family member polypeptide to the subject. The co-administration of the other FGF family member can be systemic or to the brain.

In some embodiments, the method of any one of the foregoing aspects further comprises administering one or more agents selected from the group consisting of an anti-inflammatory agent, an anti-fibrotic agent, an anti-hypertensive agent, an anti-diabetic agent, a triglyceride lowering agent, and a cholesterol lowering agent to the subject.

In some embodiments, the anti-diabetic agent is selected from the group consisting of insulin, an insulin sensitizer, an insulin secretagogue, an alpha-glucosidase inhibitor, an amylin agonist, a dipeptidyl-peptidase 4 (DPP-4) inhibitor, meglitinide, sulphonylurea, Metformin, a glucagon-like peptide (GLP) agonist or a peroxisome proliferator-activated receptor (PPAR)-gamma agonist.

In some embodiments, the PPAR-gamma agonist is a Thiazolidinedione (TZD), aleglitazar, farglitazar, tesaglitazar, or muraglitazar.

In some embodiments, the TZD is troglitazone, pioglitazone, rosiglitazone or rivoglitazone.

In some embodiments, the Glucagon-like peptide (GLP) agonist is Liraglutide, Exenatide or Taspoglutide.

In some embodiments, the subject is a mammal.

In some embodiments, the subject is a human.

In some embodiments, the blood glucose levels are lowered to normal range in 6 hours or less after a single administration of the pharmaceutical composition.

In some embodiments, the blood glucose levels are normalized in 24 hours or less after a single administration the pharmaceutical composition.

In some embodiments, the blood glucose levels are normalized in 1 week or less after a single administration of the pharmaceutical composition.

In some embodiments, the FGF1 polypeptide comprised by the pharmaceutical composition is a human FGF1 polypeptide.

In some embodiments, the FGF1 polypeptide has at least 95% amino acid sequence identity to SEQ ID NO:1 and retains at least 80% of the biological activity of human FGF1 of SEQ ID NO: 1.

In some embodiments, the FGF1 polypeptide is a human recombinant polypeptide.

In some embodiments, the FGF1 polypeptide comprises amino acids 1-155 of SEQ ID NO: 1.

In some embodiments, the FGF1 polypeptide comprises at least amino acids 25-155 of SEQ ID NO: 1.

In some embodiments, the FGF1 polypeptide preparation comprises a carrier peptide or lipophilic molecular group and/or is encapsulated in a liposome or a nanoparticle.

In one aspect, the technology described herein relates to a method of treating diabetes in a subject, the method comprising administering a single unit dose of a pharmaceutical composition comprising a Fibroblast Growth Factor 1 (FGF1) polypeptide preparation to the brain of a subject having diabetes, wherein blood glucose levels are normalized for at least 18 weeks.

In another aspect, the technology described herein relates to a method of treating elevated blood glucose levels in a subject in need thereof, comprising administering an FGF1 polypeptide to the brain of the subject, whereby blood glucose levels are lowered to a normal range.

In another aspect, the technology described herein relates to a method to induce sustained diabetes remission in a subject in need thereof, comprising administering an FGF1 polypeptide to the brain of the subject.

In another aspect, the technology described herein relates to a method to treat high blood glucose levels in a subject in need thereof, comprising administering a therapeutically effective amount of an FGFR binding protein to the brain of the subject to normalize the blood glucose levels to within the normal range, wherein the FGFR is selected from the group, FGFR1, FGFR2, FGFR3, FGFR4 or a combination thereof.

In some embodiments of the above noted aspects, the FGFR binding protein is an FGF1 polypeptide.

In one aspect, the technology described herein relates to a method of treating diabetes in a subject, comprising administering to a subject having diabetes an FGF1 polypeptide composition as described herein.

In one aspect, the technology described herein relates to a pharmaceutical composition comprising a unit dose of a FGF1 polypeptide preparation for use in the treatment of a metabolic disorder, wherein the composition is formulated for delivery to the brain, wherein the unit dose of a FGF1 polypeptide is 100 μg or less.

In one aspect, the technology described herein relates to a pharmaceutical composition comprising a unit dose of a FGF1 polypeptide preparation for use in the treatment of a metabolic disorder, wherein the composition is formulated for delivery to the brain, wherein the unit dose of a FGF1 polypeptide is less than 50% of the unit dose required to normalize blood glucose when a FGF1 polypeptide is administered systemically.

In some embodiments of any one of the foregoing aspects, the metabolic disorder is selected from the group consisting of type 2 diabetes, gestational diabetes, drug-induced diabetes, high blood glucose, insulin resistance and metabolic syndrome.

In some embodiments, the composition is formulated for administration via an intracerebroventricular, intranasal, intracranial, intracelial, intracerebellar, or intrathecal administration route.

In some embodiments, the pharmaceutical composition of any one of the foregoing aspects further comprises another FGF family member polypeptide.

In some embodiments of any one of the foregoing aspects, the FGF1 polypeptide is a human FGF1 polypeptide.

In some embodiments, the FGF1 polypeptide has at least 95% amino acid sequence identity to SEQ ID NO:1 and retains at least 80% of the biological activity of human FGF1 of SEQ ID NO: 1.

In some embodiments, the FGF1 polypeptide is a human recombinant polypeptide.

In some embodiments, the FGF1 polypeptide comprises amino acids 1-155 of SEQ ID NO: 1.

In some embodiments, the FGF1 polypeptide comprises at least amino acids 25-155 of SEQ ID NO: 1.

In some embodiments, the pharmaceutical composition for use of any one of the foregoing aspects is contained in a delivery device selected from the group consisting of a syringe, a blunt tip syringe, a catheter, an inhaler, a nebulizer, a nasal spray pump, a nasal irrigation pump or nasal lavage pump, and an implantable pump.

In some embodiments of any of the foregoing aspects, the FGF1 polypeptide is formulated with a lipophilic molecular group.

In some embodiments, the FGF1 polypeptide is encapsulated in a liposome or a nanoparticle.

In some embodiments, the FGF1 polypeptide is fused to a carrier polypeptide.

In one aspect the technology described herein relates to a pharmaceutical composition formulated for intranasal administration to a subject in need thereof, comprising a unit dose of a Fibroblast Growth Factor 1 (FGF1) polypeptide preparation in combination with a ganglioside and/or a phosphotidylserine, wherein the unit dose of a Fibroblast Growth Factor 1 (FGF1) polypeptide is 100 μg or less.

In one aspect the technology described herein relates to a pharmaceutical composition formulated for intranasal administration to a subject in need thereof, comprising a unit dose of a Fibroblast Growth Factor 1 (FGF1) polypeptide preparation in combination with a ganglioside and/or a phosphotidylserine, wherein the unit dose of a Fibroblast Growth Factor 1 (FGF1) polypeptide is less than 50% of the unit dose required to transiently normalize blood glucose when an FGF1 polypeptide is administered systemically.

In one aspect the technology described herein relates to a pharmaceutical composition formulated for intranasal administration to a subject in need thereof, comprising a unit dose of a Fibroblast Growth Factor 1 (FGF1) polypeptide preparation in combination with a saccharide selected from the group consisting of cyclodextrins, disaccharides, polysaccharides, and combinations thereof, and wherein the unit dose of an FGF1 polypeptide is 100 μg or less.

In one aspect the technology described herein relates to a pharmaceutical composition formulated for intranasal administration to a subject in need thereof, comprising a unit dose of a Fibroblast Growth Factor 1 (FGF1) polypeptide preparation in combination with a saccharide selected from the group consisting of cyclodextrins, disaccharides, polysaccharides, and combinations thereof, and wherein the unit dose of an FGF1 polypeptide is less than 50% of the unit dose required to transiently normalize blood glucose when an FGF1 polypeptide is administered systemically.

In one aspect the technology described herein relates to a method of treating diabetes in a subject who has a blood glucose level greater than or equal to 300 mg/dL prior to treatment, the method comprising administering insulin and then administering a single dose FGF1 polypeptide preparation to the brain, wherein blood glucose levels are normalized for at least 1 week.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of” refers to those elements required for a given embodiment. The term permits the presence of elements that do not materially affect the basic and novel or functional characteristic(s) of that embodiment of the invention.

As used herein the term “consisting of” refers to compositions, methods, and respective components thereof as described herein, which are exclusive of any element not recited in that description of the embodiment.

The terms “disease”, “disorder”, or “condition” are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

The term “in need thereof” when used in the context of a therapeutic or prophylactic treatment, means having a disease, being diagnosed with a disease, or being in need of preventing a disease, e.g., for one at risk of developing the disease. Thus, a subject in need thereof can be a subject in need of treating or preventing a disease.

As used herein, the terms “treat,” “treatment,” “treating,” or “amelioration” refer to therapeutic treatments, wherein the object is to reverse, alleviate, ameliorate, inhibit, slow down or stop the progression or severity of a metabolic disorder or syndrome, e.g., Diabetes mellitus (DM), type 2 diabetes or other disorder characterized by or involving blood glucose dysregulation. The term “treating” includes reducing or alleviating at least one adverse effect or symptom of a metabolic syndrome. Treatment is generally “effective” if one or more symptoms or clinical markers are reduced. In the case of abnormally high blood glucose or diabetes, “effective treatment” refers to a treatment that reduces hyperglycemia to the normal blood sugar range and maintains it within the normal range for at least one week. Treatments described herein can reduce hyperglycemia and maintain normal ranges of blood sugar for at least two weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, or more, e.g, at least 20 weeks (or 5 months), 6 months or more. Alternatively, or in addition, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality. For example, treatment is considered effective if the condition is stabilized, or the elevated blood glucose levels are normalized. The term “treatment” of a disease also includes providing relief from the symptoms or side-effects of the disease (including palliative treatment).

As used herein, the term “administering,” refers to the placement of a compound as disclosed herein into a subject by a method or route that results in at least partial delivery of the agent at a desired site. Pharmaceutical compositions comprising the compounds disclosed herein can be administered by any appropriate route which results in an effective treatment in the subject, e.g., intracerebroventricular (“icv”) administration, intranasal administration, intracranial administration, intracelial administration, intracerebellar administration, or intrathecal administration

As used herein, a “subject”, “patient”, “individual” and like terms are used interchangeably and refers to a vertebrate, preferably a mammal, more preferably a primate, still more preferably a human. Mammals include, without limitation, humans, primates, rodents, wild or domesticated animals, including feral animals, farm animals, sport animals, and pets. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, and canine species, e.g., dog, fox, wolf, avian species, e.g., chicken, emu, ostrich, and fish, e.g., trout, catfish and salmon. The terms, “individual,” “patient” and “subject” are used interchangeably herein. A subject can be male or female.

Preferably, the subject is a mammal. The mammal can be a human, non-human primate, mouse, rat, dog, cat, horse, or cow, but is not limited to these examples. Mammals other than humans can be advantageously used as subjects that represent animal models of conditions or disorders associated with diabetes. Such models are known in the art and are described in (24). Non-limiting examples include the Lep^{ob/ob} murine model, the Lepr^{db/db} murine model, and the streptozocin-induced diabetes model. In addition, the compositions and methods described herein can be used to treat domesticated animals and/or pets.

A subject can be one who has been previously diagnosed with or identified as suffering from or under medical supervision for a metabolic disorder. A subject can be one who is diagnosed and currently being treated for, or seeking treatment, monitoring, adjustment or modification of an existing therapeutic treatment, or is at a risk of developing a metabolic disorder, e.g., due to sedentary lifestyle, family history etc.

As used herein, the terms “protein”, “peptide” and “polypeptide” are used interchangeably to designate a series of amino acid residues connected to each other by peptide bonds between the alpha-amino and carboxy groups of adjacent residues. The terms “protein”, “peptide” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein”, “peptide” and “polypeptide” are used interchangeably herein when referring to a gene product and fragments thereof.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically acceptable carrier” means a pharmaceutically-acceptable material, composition or vehicle, such as a liquid or solid filler, diluent, excipient, manufacturing aid or solvent encapsulating material necessary or used in formulating an active ingredient or agent for delivery to a subject. Each carrier must be “acceptable” in the sense of being compatible with the other ingredients of the formulation and not injurious to the patient.

The term “unit dose” described herein is defined as a unit containing a predetermined quantity of active material calculated to produce the desired therapeutic effect in association with the required diluent i.e. a carrier or vehicle. The stated amounts of active material, for example a polypeptide, refers to the weight of polypeptide without the carrier, when a carrier is used. The unit dose can be a physically discrete unit suitable as unitary dosages for animals. The specifications for the unit dose for embodiments described herein are dictated by and are directly dependent on (a) the unique characteristics of the active material and the particular therapeutic effect to be achieved, and (b) the limitations inherent in the art of compounding such active material for anti-diabetic use in animals, via administration to the brain as disclosed in detail herein, these being features of the embodiments described herein. A unit dose for example, contains the principal active ingredient, FGF1 polypeptide, in amounts ranging from 250 μg to 5 μg. The unit dose is further defined as the dose, containing the principal active ingredient, FGF1 polypeptide, required to produce the desired therapeutic effect of prolonged lowering or normalization of blood glucose levels upon administration to the brain and is lower than that required for similar, albeit transient blood-glucose normalizing effect when administered systemically. For example, the unit dose of FGF1 polypeptide can be less than 50% of that needed to be effective when administered systemically, preferably less than 40%, less than 30%, less than 25%, less than 20%, less than 15%, or 10% or lower relative to the dose required for transient systemic blood glucose-lowering effect.

The term “therapeutically effective amount” as used herein refers to an amount sufficient to effect a beneficial or desired clinical result upon treatment. Specifically, the term “therapeutically effective amount” means an amount of an FGF1 polypeptide-containing composition as described herein sufficient to measurably lower or normalize elevated blood glucose levels without causing hypoglycemia in a relevant blood glucose monitoring assay, or sufficient to cause a measurable improvement in an animal model of metabolic syndrome and/or diabetes. Alternatively, a “therapeutically effective amount” is an amount of an FGF polypeptide-containing composition described herein sufficient to confer a therapeutic or prophylactic effect on the subject treated for metabolic syndrome, diabetes or other disorder involving or characterized by abnormally high blood sugar. In some embodiments, a therapeutically effective amount of an FGF1 polypeptide composition is formulated in a single unit dose, which is effective for administration to the brain and sufficient to normalize blood glucose levels for an extended or prolonged period with administration of the single unit dose.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents. Furthermore, therapeutically effective amounts will vary, as recognized by those skilled in the art, depending on the specific disease treated, the route of administration, the excipient selected, and the possibility of combination therapy.

Physiological effects that can be measured to determine therapeutic effect and/or the therapeutically effective amount include, without limitation, lowering of blood glucose levels, changes in insulin sensitivity, insulin secretion, body weight and food intake. Relevant assays to measure such effects include, without limitation, measurement of fasting blood glucose levels and the oral glucose tolerance test. Blood glucose can be measured in a sample of blood taken from a vein or from a small finger stick sample of blood. It can be measured in a laboratory either alone or with other blood tests, or it can be measured using a handheld glucometer, a small device that allows frequent monitoring of blood glucose levels without the need for a doctor's office or laboratory.

In certain embodiments, FGF1 polypeptide can be formulated in liposomes to promote delivery across membranes. As used herein, the term “liposome” refers to a vesicular structure having lipid-containing membranes enclosing an aqueous interior. In cell biology, a vesicular structure is a hollow, lamellar, spherical structure, and provides a small and enclosed compartment, separated from the cytosol by at least one lipid bilayer. Liposomes can have one or more lipid membranes. Oligolamellar large vesicles and multilamellar vesicles have multiple, usually concentric, membrane layers and are typically larger than 100 nm. Liposomes with several nonconcentric membranes, i.e., several smaller vesicles contained within a larger vesicle, are termed multivesicular vesicles.

Liposomes can further comprise one or more additional lipids and/or other components such as sterols, e.g., cholesterol. Additional lipids can be included in the liposome compositions for a variety of purposes, such as to prevent lipid oxidation, to stabilize the bilayer, to reduce aggregation during formation or to attach ligands onto the liposome surface. Any of a number of additional lipids and/or other components can be present, including amphipathic, neutral, cationic, anionic lipids, and programmable fusion lipids. Such lipids and/or components can be used alone or in combination. One or more components of the liposome can comprise a ligand, e.g., a targeting ligand.

Liposome compositions can be prepared by a variety of methods that are known in the art. See e.g., patents cited as reference, (25, 26, 27, 28, 29, 30). Niosomes are non-phospholipid based synthetic vesicles that have properties and function like liposomes.

As used herein, “micelles” are a particular type of molecular assembly in which amphipathic molecules are arranged in a spherical structure such that all hydrophobic portions on the molecules are directed inward, leaving the hydrophilic portions in contact with the surrounding aqueous phase. The converse arrangement exists if the environment is hydrophobic.

In some embodiments, FGF1 polypeptide formulations comprise micelles formed from lipid-associated FGF1 polypeptide, e.g., FGF1 conjugated to at least one amphiphilic carrier, in which the micelles have an average diameter of less than about 100 nm, preferably. More preferred embodiments provide micelles having an average diameter less than about 50 nm, and even more preferred embodiments provide micelles having an average diameter less than about 100 nm, or even less than about 20 nm.

As used herein, the term “nanoparticle” refers to a particle having a size between 1 and 1000 nm which can be manufactured from artificial or natural macromolecular substances. To such nanoparticles can be bound drugs or other biologically active materials by covalent, ionic or adsorptive linkage, or the latter can be incorporated into the material of the nanoparticles. Nanoparticles may or may not exhibit size-related properties that differ significantly from those observed in fine particles or bulk materials (31). Nanoparticles provide improved bioavailability by enhancing aqueous solubility, increasing resistance time in the body (increasing half-life for clearance/increasing specificity for its cognate receptors and targeting drug to specific location in the body (its site of action). This results in concomitant reduction in quantity of the drug required and dosage toxicity, enabling the safe delivery of toxic therapeutic drugs and protection of non target tissues and cells from severe side effects. As described in reference 32 and 33, non-limiting examples of nanoparticles include solid lipid nanoparticles (comprise lipids that are in solid phase at room temperature and surfactants for emulsification, the mean diameters of which range from 50 nm to 1000 nm for colloid drug delivery applications), liposomes, nanoemulsions (oil-in-water emulsions done on a nano-scale), albumin nanoparticles, and polymeric nanoparticles.

Nanoparticles can be surface coated to modulate their stability, solubility, and targeting. A coating that is multivalent or polymeric confers high stability (34). A non-limiting example includes coating with hydrophilic polymer such as polyethylene glycol or ploysorbate-80.

As used herein the term “lipophilic molecular group” refers to a lipid moiety, such as a fatty acid, glyceride or phospholipid which when coupled to a therapeutic molecule to be a targeted to the brain, increases its lipophilicity and hence movement across blood brain barrier. The lipophilic molecular group can be attached to the therapeutic molecule through an ester bond.

As used herein the term “carrier polypeptide” refers to a peptide which exhibits substantially no bioactivity and which is capable of passing the blood-brain barrier. When conjugated with a biologically active therapeutic peptide incapable of passing the blood brain barrier, the carrier polypeptide enables the uniform transport of the therapeutic peptide to the brain without any side effect of the carrier polypeptide. The carrier peptide can be an endogenous peptide whose receptor is present on the cerebral capillary endothelial cell, such as insulin, insulin-like growth factor (IGF), leptin and transferrin or fragments thereof (see, e.g., reference 35). The carrier peptide can be, for example, a short cell penetrating peptide of less than 30 amino acids that are amphipathic in nature and are able to interact with lipidic membranes. Non-limiting examples of carrier peptides include SynB3, TAT (HIV-1 transactivating transcriptor).

As used herein, the term “in combination” refers to the use of more than one prophylactic and/or therapeutic agent simultaneously or sequentially and in a manner such that their respective effects are additive or synergistic.

The terms “increased”, “increase”, or “enhance” are all used herein to generally mean an increase by a statically significant amount; for the avoidance of doubt, the terms “increased”, “increase”, or “enhance”, mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms, “decrease”, “reduce”, “reduction”, “lower” or “lowering,” or “inhibit” are all used herein generally to mean a decrease by a statistically significant amount. For example, “decrease”, “reduce”, “reduction”, or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The term “statistically significant” or “significantly” refers to statistical significance and generally means a difference of two standard deviations (2SD) or more.

A normal fasting (no food for 8 hours) blood glucose level is between 70 and 100 mg/dL—this is a fasting blood glucose level “within the normal range” as the term is used herein. Blood glucose levels will rise after food is ingested, but will normally be less than 140 mg/dL two hours after eating. A fasting blood glucose level between 100 and 125 mg/dL or any value between 140 and 199 mg/dL during a two hour 75 g oral glucose tolerance test is considered to be a marker of pre-diabetes and constitutes an “elevated,” “abnormally high” or “abnormally elevated” blood glucose level, also referred to herein as “hyperglycemia” or a level “above the normal range.” An individual is considered diabetic (and also to have an “elevated,” “abnormally high” or “abnormally elevated” or “hyperglycemic” blood glucose level) if they have two consecutive fasting blood glucose tests greater than 126 mg/dL, any random blood glucose test level greater than 200 mg/dL, or a two hour 75 g oral glucose tolerance test with any level over 200 mg/dL.

The term “normalizing” refers to a change in blood glucose levels to within the normal range from an elevated or hyperglycemic level, without becoming hypoglycemic. “Normalizing” refers not only to the activity of promoting a decrease in an abnormally high blood glucose level, but also maintaining such levels for a prolonged period of time, e.g., at least one week for a single unit dose pharmaceutical composition administration as described herein.

The term “hypoglycemia” refers to a condition displaying lower blood glucose levels than those accepted as within the normal range.

The term “anti-inflammatory agent” refers to an agent (e.g., a small molecule compound, a protein) that blocks, inhibits, or reduces inflammation or signaling from an inflammatory signaling pathway. Non-limiting example include IL-1 or IL-1 receptor antagonist, such as anakinra (KINERET®), rilonacept, or canakinumab, anti-TNFα antibody, such as infliximab (REMICADE®), golimumab (SIMPONI®), adalimumab (HUMIRA®), certolizumab pegol (CIMZIA®) or etanercept.

The term “anti-fibrotic agent” refers to an agent (e.g., a small molecule compound, a protein) that blocks, inhibits, or reduces fibrosis or tissue scarring.

The term “anti-hypertensive agent” refers to an agent (e.g., a small molecule compound, a protein) that reduces high blood pressure when administered to a patient (e.g., a hypertensive patient). Exemplary anti-hypertensive agents, include but are not limited to, renin angiotensin aldosterone system antagonists (“RAAS antagonists”), angiotensin converting enzyme (ACE) inhibitors, and angiotensin II receptor blockers (AT₁ blockers).

The term “anti-diabetic agent” refers to an agent (e.g., a small molecule compound, a protein) other than an FGF1 polypeptide as described herein, that lowers blood glucose level to a normal range and relieves diabetes symptoms such as thirst, polyuria, weight loss and/or ketoacidosis. In the long-term, such an agent can prevent the development of or slow the progression of long term complications of the disease, such as kidney disease, high blood pressure and/or stroke when administered to a patient (e.g., a diabetic patient). Non-limiting examples include insulin and oral medications such as thiazolidinediones, metformin and liraglutide.

A triglyceride lowering agent (e.g., a small molecule compound, a protein) refers to an agent that lowers triglyceride level to a normal level below 100 milligrams per deciliter of blood. In the long-term, such agents can prevent the development of or slow the progression of long term complications of the disease such as heart disease, obesity and metabolic syndrome. Non-limiting examples include niacin, fibrates and statins.

A cholesterol lowering agent (e.g., a small molecule compound, a protein) refers to agents that lower blood cholesterol levels to typical normal levels of less than 200 mg/dL of total cholesterol and less than 100 mg/dL of LDL cholesterol levels. Non limiting examples include statins, bile-acid-binding resins and cholesterol absorption inhibitors.

As used herein the term “insulin sensitizer” refers to an agent (e.g., a small molecule compound, a protein) that improves the sensitivity of cells to the metabolic effects of insulin when administered to a patient (e.g., patient with insulin resistance, diabetes). Non limiting examples of insulin sensitizers include thiazolidinediones and metformin.

As used herein the term “insulin secretagogue” refers to an agent that increase insulin release from beta cells in the pancreas when administered to a patient (e.g., a type 2 diabetes patient). Non limiting examples of insulin secretagogue include sulphonylurea, meglitinides and glucagon-like peptide.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other than in the operating examples, or where otherwise indicated, all numbers expressing quantities of ingredients or reaction conditions used herein should be understood as modified in all instances by the term “about.” The term “about” when used in connection with percentages means ±1% of the value being referred to. For example, about 100 means from 99 to 101.

Although methods and materials similar or equivalent to those described herein can be used in the practice or testing of this disclosure, suitable methods and materials are described below. The abbreviation, “e.g.,” is derived from the Latin exempli gratia, and is used herein to indicate a non-limiting example. Thus, the abbreviation “e.g.,” is synonymous with the term “for example.”

As used in this specification and appended claims, the singular forms “a,” “an”, and “the” include plural references unless the context clearly dictates otherwise. Thus for example, reference to “the method” included one or more methods, and/or steps of the type described herein and/or which will become apparent to those persons skilled in the art upon reading this disclosure and so forth.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

### BRIEF DESCRIPTION OF THE DRAWINGS

FIGS. 1A-1I Sustained glucose lowering induced by a single icv FGF1 injection in ob/ob mice. Blood glucose (BG) levels during an ipGTT performed in fasted ob/ob (B6) mice 6 h after (FIG. 1A) a single icv injection of either vehicle (Veh; open symbols; n=8) or 3 μg of mFGF1 (black symbols: n=9), or (FIG. 1B) a single sc injection of either Veh or the same dose of mFGF1 (Veh, n=7; FGF1, n=6). (FIGS. 1C-1E) BG values from an ipGTT performed in fasted ob/ob (B6) mice either 7 d (FIG. 1C), 4 wk (FIG. 1D), or 18 wk (FIG. 1E) following a single icv injection of mFGF1 (3 μg). (FIG. 1F) Time course of BG levels from the same cohort of ad-libitum (ad-lib)-fed ob/ob mice both prior to and after a single icv injection of mFGF1 (3 μg). (FIG. 1G) Body weight, (FIG. 1H) fat mass, and (FIG. 1I) food intake of ob/ob (B6) mice following icv injection of either mFGF1 or Veh. Data are the mean±s.e.m. P-values for group (Veh vs. FGF1) computed by repeated measures designs by linear mixed model analyses and within time-point 95% confidence intervals for group differences shown in FIG. 3.

FIGS. 2A-2C Effect of icv FGF1 on glucoregulatory hormones in diabetic mice. Plasma levels of (FIG. 2A) insulin, (FIG. 2B) glucagon and (FIG. 2C) corticosterone in ob/ob (B6) mice 18 wk after a single icv injection of either mFGF1 (3 μg; black bars, n=9) or Veh (open bars, n=8). Data are mean±s.e.m. P=ns, icv mFGF1 vs. Veh as determined by two-tailed t-test.

FIGS. 3A-3F 95% confidence intervals for FGF1 minus Veh group differences in BG induced by single icv FGF1 injection in ob/ob mice. ipGTT BG differences in fasted ob/ob (B6) mice, 6 h after receiving either a single icv injection (FIG. 3A) (Veh, n=8, mFGF1, n=9), or sc injection (FIG. 3B) (Veh, n=7, mFGF1, n=6) of mFGF1 (3 μg). (FIG. 3C-3E) ipGTT BG differences in samples from fasted ob/ob (B6) mice measured (FIG. 3C) 1 wk, (FIG. 3D) 4 wk, and (e) 18 wk following a single icv injection of mFGF1 (3 μg) or Veh. (FIG. 3F) Differences in basal ad-lib-fed BG obtained from ob/ob (B6) mice after a single icv injection of mFGF1. Intervals that exclude zero correspond to P<0.05, two-tailed. Statistics by independent groups t-tests.

FIGS. 4A-4F Diabetes remission induced by a single icv FGF1 injection across multiple murine models of T2D. (FIG. 4A) Daily BG levels from ad-lib-fed ob/ob (B6) mice following a single icv injection of either mFGF1 (3 μg; n=6; black symbols), hFGF1 (3 μg; n=6; grey symbols) or Veh (n=4; open symbols). (FIG. 4B) Fasting BG values from lean, WT mice 6 h after icv injection of either mFGF1 (3 μg; n=5) or Veh (n=5). (FIG. 4C) BG values from ad-lib-fed ob/ob (B6) mice following a single sc injection of either mFGF1 (0.5 mg/kg body weight; n=11) or Veh (n=10). (FIG. 4D) Fasting (0 and 90 min) and ad-lib-fed BG levels (Day 5) following icv injection of FGF19 (3 μg; n=5) or Veh (n=5) in ob/ob (B6) mice. (FIG. 4E) Time course of BG levels from ad-lib-fed db/db mice both prior to and following a single icv injection of mFGF1 (3 μg; n=6) or Veh (n=9). (FIG. 4F) Time course of BG levels from ad-lib-fed DIO WT mice rendered diabetic with a low dose of STZ (DIO-LD STZ) both prior to and following a single icv injection of mFGF1 (3 μg; n=3) or Veh (n=4). Data are mean±s.e.m. P-values for group (Veh vs. FGF1) by repeated measures designs by linear mixed model analyses and within time-point 95% confidence intervals for group differences shown in FIG. 5. *P<0.05, FGF1 (or FGF19) vs. Veh as determined by two-tailed t-test.

FIGS. 5A-5D 95% confidence intervals for FGF1 minus veh group differences in BG induced by single icv FGF1 injection across multiple murine models of T2D. (FIG. 5A) Differences in BG in samples from ob/ob (B6) mice fed ad-lib following a single icv injection (3 μg) of either mFGF1 or hFGF1 (combined n=12) or Veh (n=4). (FIG. 5B) Fasting BG differences from WT mice 6 h after icv injection of either mFGF1 (3 μg) (n=5) or Veh (n=5). (FIG. 5C) BG differences from db/db mice fed ad-lib following a single icv mFGF1 (3 μg; n=6) or Veh (n=9). (FIG. 5D) BG differences from DIO-LD STZ mice fed ad-lib following a single icv mFGF1 (3 μg; n=3) or Veh (n=4). Intervals that exclude zero correspond to P<0.05, two-tailed. Statistics by independent groups t-tests.

FIGS. 6A-6F Effect of icv FGF1 on food intake and body weight across multiple murine models of T2D. Time course of changes of (FIG. 6A) food intake and (FIG. 6B) body weight of ob/ob (B6) mice following icv injection of mFGF1 (3 μg; black symbols; n=6), hFGF1 (3 μg; grey symbols; n=6) or Veh (open symbols; n=4). Time course of changes of (FIG. 6C) food intake and (FIG. 6D) body weight in db/db mice following icv injection of either mFGF1 (3 μg; n=6) or Veh (n=9). Time course of changes of (FIG. 6E) food intake and (FIG. 6F) body weight in DIO WT mice treated with low dose STZ (DIO-LD STZ) following icv injection of either mFGF1 (3 μg; n=3) or Veh (n=4). Data are mean±s.e.m. *P<0.05, icv mFGF1 vs. Veh. ^{#}P<0.05, icv hFGF1 vs. Veh by mixed factorial analyses.

FIGS. 7A-7D The anti-diabetic effect of a single icv FGF1 injection is reproducible in a rat model of T2D. (FIG. 7A) Daily BG levels from ad-lib-fed ZDF rats following a single icv injection of either rFGF1 (3 μg; n=10; black symbols) or Veh (n=10; open symbols). (FIG. 7B) Body weight, (FIG. 7C) food intake, and (FIG. 7D) fat mass of ZDF rats following icv injection of either rFGF1 or Veh. Data are the mean±s.e.m. P-values for group (Veh vs. FGF1) by repeated measures designs by linear mixed model analyses and within time-point 95% confidence intervals for group differences shown in FIG. 8. Significant main effects in b (P=0.028) and c (P<0.0001) reflected group differences at earlier time points (treatment by day interaction is significant (P<0.0001) in b, c; see FIG. 8).

FIGS. 8A-8C 95% confidence intervals for FGF1 minus veh group differences in BG, body weight, and food intake induced by single icv FGF1 injection in a rat model of T2D. Differences in BG levels (FIG. 8A), body weight (BW) (FIG. 8B), and food intake (FI) (FIG. 8C) in ZDF rats fed ad-lib following a single icv injection (3 μg) of either rFGF1 (n=10) or Veh (n=10). Intervals that exclude zero correspond to P<0.05, 2-tailed. Statistics by independent groups t-tests.

FIGS. 9A-9I Effect a single icv injection of FGF1 on whole-body glucose kinetics in ob/ob mice. ob/ob (B6) mice underwent a basal glucose turnover study followed by a frequently sampled intravenous glucose tolerance test (FSIGT) 7 d after a single icv injection of mFGF1 (3 μg, black symbols; n=13) or Veh (open symbols; n=9). (FIG. 9A) Mean basal glucose turnover rate (GTR); (FIG. 9B) basal glucose clearance rate. (FIG. 9C) Fasting BG levels, and (FIG. 9D) delta area under the glucose curve (A AUC) during the FSIGT (after correcting for differences of basal glucose). (FIG. 9E) Plasma insulin levels, and (FIG. 9F) the acute insulin response to glucose (AIR_{g}) during the FSIGT. (FIG. 9G) Liver glycogen content and (FIG. 9H) levels of mRNA encoding liver glucoregulatory genes from samples obtained at study termination. (FIG. 9I) Basal plasma lactate levels obtained prior to the FSIGT. Data are mean±s.e.m. *P<0.05, FGF1 vs. Veh as determined by two-tailed t-test.

FIGS. 10A-10I Effect of icv FGF1 on insulin sensitivity, insulin-independent glucose disposal and plasma lipid levels. (FIG. 10A) Insulin sensitivity (S_{I}), (FIG. 10B) insulin-independent glucose disposal (S_{G}), and (FIG. 10C) brown adipose tissue (BAT) UCP-1 gene expression in ob/ob (B6) mice that underwent a basal glucose turnover study followed by a FSIGT 7 d after a single icv injection of mFGF1 (3 μg, black symbols; n=13) or Veh (open symbols; n=9). Plasma levels of (FIG. 10D) triglyceride (TG), (FIG. 10E) cholesterol (Chol) and (FIG. 10F) non-esterified free fatty acids (NEFA) in ob/ob (B6) mice on samples obtained 28 d following a single icv injection of mFGF1 (3 μg; black symbols; n=6), hFGF1 (3 μg; grey symbols; n=6) or Veh (open symbols; n=4). (FIG. 10G) Plasma levels of plasma TG, (FIG. 10H) Chol, and (FIG. 10I) NEFA from db/db mice on samples obtained 28 d following a single icv injection of either mFGF1 (3 μg; n=6) or Veh (n=9). Data are mean±s.e.m. ^{#}P<0.05, icv hFGF1 vs. Veh as determined by one-way ANOVA.

FIGS. 11A-11F Requirement for intact basal insulin signaling in central FGF1-mediated glucose lowering. (FIG. 11A) Time course of BG levels in more severely hyperglycemic, ad-lib-fed ob/ob (BTBR) mice following icv injection of mFGF1 (black symbols; n=8) or Veh (open symbols; n=8). (FIG. 11B) Time course of BG levels in more severely hyperglycemic, ad-lib-fed db/db following icv injection of mFGF1 (black symbols; n=4) or Veh (open symbols; n=9). (FIG. 11C) Time course of BG levels in more severely hyperglycemic, ad-lib-fed DIO WT mice treated with high dose-STZ (DIO-HD STZ) following icv injection of mFGF1 (black symbols; n=4) or Veh (open symbols; n=3). (FIG. 11D) Food intake; (FIG. 11E) body weight and (FIG. 11F) BG levels from ad-lib-fed DIO WT mice receiving continuous sc infusion of the insulin receptor antagonist S961 that received icv injection of either Veh (open symbols; n=10) or mFGF1 (3 μg; black symbols; n=11). Data are the mean±s.e.m. For FIG. 11D, the groups differed on Days 1 and 2 (P<0.0001 and P=0.043, respectively). *P-values for group (Veh vs. FGF1) by repeated measures designs by linear mixed model analyses and within time-point 95% confidence intervals for group differences shown in FIG. 12.

FIG. 12 95% confidence interval for FGF1 minus Veh group differences in food intake induced by a single icv FGF1 injection in ad-lib-fed DIO WT mice receiving continuous sc infusion of the insulin receptor antagonist S961. Differences in food intake from ad-lib-fed DIO WT mice receiving continuous sc infusion of the insulin receptor antagonist S961 that received icv injection of either Veh (n=10) or mFGF1 (3 μg; n=11). Intervals that exclude zero correspond to P<0.05, two-tailed. Statistics by independent groups t-tests.

FIGS. 13A-13B In normal, non-diabetic mice, icv FGF1 does not cause side effects associated with insulin therapy such as hypoglycemia (FIG. 13A) or weight gain (FIG. 13B).

### DETAILED DESCRIPTION

Described herein are compositions and methods to treat metabolic disorders involving abnormally elevated blood glucose levels by administration of FGF1 polypeptide to the brain. The inventors have shown that as little as one intracerebroventricular administration of FGF1 normalizes blood glucose levels and induces prolonged diabetes remission compared to that induced by its systemic administration. The prolonged blood glucose normalization and/or diabetes remission induced by administration of FGF1 to the brain can be induced by a dosage that is 10-fold lower than that required to achieve transient blood glucose reduction via systemic administration. Furthermore, as opposed to blood glucose normalization and/or anti-diabetic effect by systemic administration of FGF1, the effect of FGF1 administration to the brain is independent of significant changes in insulin sensitivity, basal insulin levels or glucose-induced insulin secretion. Without wishing to be bound by theory, the effect is believed to involve changes in basal glucose clearance. Unlike current diabetes treatment strategies, FGF1-based therapeutic administration to the brain does not induce hypoglycemia or lasting changes in body weight or food intake. The various considerations for one of skill in the art to make the compositions and perform the methods necessary to treat metabolic disorders via administration of FGF1 polypeptides to the brain are described herein below.

### Fibroblast Growth Factors

Fibroblast growth factors (FGFs) form a family of generally extracellular signaling polypeptides, which are key regulators of a number of biological processes. In humans, the FGF family includes 22 known members (FGF-1 to 14 and FGF-16 to 23), which are further divided into subfamilies according to their sequence homology and function (36). FGFs are small proteins (between 17 and 34 kDa) characterized by a relatively well conserved central domain of 120 to 130 amino acids. This domain is organized into 12 antiparallel β sheets forming a triangular structure referred to as a beta trefoil. Some FGFs possess significant extensions, either C-terminal, N-terminal, or both, outside of this core sequence. All FGFs, with the exception of the intracellular FGFs (iFGFs, FGF11-14), signal through a family of tyrosine kinase receptors, the FGF receptors (FGFRs). Two FGF ligands bind a dimeric receptor in the presence of heparan sulfate proteoglycan (HSPG), allowing the transphosphorylation and activation of the intracellular tyrosine kinase domain of the receptor. Binding of FGF polypeptides to FGFRs usually activates several intracellular cascades (i.e., Ras/MAPK, PI3K/Akt, and PLC/PKC), which can regulate the transcription of different target genes. Intracellular FGFs (iFGFs, FGF11-14), also known as FGF homologous factors 1-4 (FHF1-FHF4), have been shown to have distinct functions compared to the FGFs. Although these factors possess remarkably similar sequence homology, they do not bind FGFRs.

### Fibroblast Growth Factor-1 (FGF-1)

Fibroblast growth factor-1 (FGF-1), also known as acidic FGF1, is a 155 amino acid polypeptide growth factor involved in the regulation of diverse physiological processes such as development, angiogenesis, wound healing, adipogenesis, and neurogenesis. As used herein, the term “Fibroblast Growth Factor 1 polypeptide” or “FGF1 polypeptide” refers to a full length FGF1 polypeptide or to a fragment or derivative thereof that retains the ability, at a minimum, to reduce or normalize elevated blood sugar when administered to the brain of a subject with abnormally elevated blood sugar or diabetes. As demonstrated herein, the effect of central administration of FGF1 polypeptides is consistent in different animal models of diabetes, including in the ob/ob diabetic mouse model, the db/db mouse model and the streptozocin-induced diabetes model—an FGF1 polypeptide or polypeptide fragment that retains the ability to reduce abnormally high blood glucose levels when administered to the brain in humans or in any of these models is an “FGF1 polypeptide” or a “functional FGF1 polypeptide” as those terms are used herein.

The FGF1 polypeptide of the compositions and methods described herein can be full length human FGF1 and/or functional fragments thereof, a species homologue and/or functional fragments thereof, an ortholog of human FGF1 and/or functional fragments thereof. The FGF1 polypeptide can be a mammalian FGF1 polypeptide. The FGF1 polypeptide can also be a functional isoform of the full length FGF1 or functional fragment thereof.

In some embodiments, the FGF1 polypeptide includes or is derived from human FGF1 having the following amino acid sequence (SEQ ID NO:1).

| | |
|---|---|
| 1 | MAEGEITTFT ALTEKFNLPP GNYKKPKLLY CSNGGHFLRI LPDGTVDGTR DRSDQHIQLQ |
| 61 | LSAESVGEVY IKSTETGQYL AMDTDGLLYG SQTPNEECLF LERLEENHYN TYISKKHAEK |
| 121 | NWFVGLKKNG SCKRGPRTHY GQKAILFLPL PVSSD |

(See GenBank Accession No. AAH32697, which is incorporated herein by reference in its entirety).

The polypeptide and coding nucleic acid sequences of FGF1 and of other members of the family of human origin and those of a number of animals are publically available, e.g., from the NCBI website. Examples include, but are not limited to,

| SEQ ID NO: 1 | |
|---|---|
| FGF1 protein [Homo sapiens] | |
| GenBank: AAH32697.1 | |
| 1 | MAEGEITTFT ALTEKFNLPP GNYKKPKLLY CSNGGHFLRI LPDGTVDGTR DRSDQHIQLQ |
| 61 | LSAESVGEVY IKSTETGQYL AMDTDGLLYG SQTPNEECLF LERLEENHYN TYISKKHAEK |
| 121 | NWFVGLKKNG SCKRGPRTHY GQKAILFLPL PVSSD |
| SEQ ID NO: 2 | |
| FGF1 protein [Homo sapiens] | |
| Amino acid residue 25-155 of SEQ ID NO: 1 | |
| 1 | KPKLLY CSNGGHFLRI LPDGTVDGTR DRSDQHIQLQ |
| 37 | LSAESVGEVY IKSTETGQYL AMDTDGLLYG SQTPNEECLF LERLEENHYN TYISKKHAEK |
| 97 | NWFVGLKKNG SCKRGPRTHY GQKAILFLPL PVSSD |
| SEQ ID NO: 3 | |
| FGF1 protein [Homo sapiens] | |
| Amino acid residue 29-155 OF SEQ ID NO: 1 | |
| 1 | LY CSNGGHFLRI LPDGTVDGTR DRSDQHIQLQ |
| 33 | LSAESVGEVY IKSTETGQYL AMDTDGLLYG SQTPNEECLF LERLEENHYN TYISKKHAEK |
| 93 | NWFVGLKKNG SCKRGPRTHY GQKAILFLPL PVSSD |
| SEQ ID NO: 4 | |
| Fgf1 protein [Mus musculus] | |
| GenBank: AAH37601.1 | |
| 1 | MAEGEITTFA ALTERFNLPL GNYKKPKLLY CSNGGHFLRI LPDGTVDGTR DRSDQHIQLQ |
| 61 | LSAESAGEVY IKGTETGQYL AMDTEGLLYG SQTPNEECLF LERLEENHYN TYTSKKHAEK |
| 121 | NWFVGLKKNG SCKRGPRTHY GQKAILFLPL PVSSD |
| SEQ ID NO: 5 | |
| FGF1 protein [Rattus norvegicus] | |
| UniProtKB/Swiss-Prot: P61149.1 | |
| 1 | MAEGEITTFA ALTERFNLPL GNYKKPKLLY CSNGGHFLRI LPDGTVDGTR DRSDQHIQLQ |
| 61 | LSAESAGEVY IKGTETGQYL AMDTEGLLYG SQTPNEECLF LERLEENHYN TYTSKKHAEK |
| 121 | NWFVGLKKNG SCKRGPRTHY GQKAILFLPL PVSSD |
| SEQ ID NO: 6 | |
| FGF1 protein [Bos taurus] | |
| GenBank: AAI03226.1 | |
| 1 | MAEGETTTFT ALTEKFNLPL GNYKKPKLLY CSNGGYFLRI LPDGTVDGTK DRSDQHIQLQ |
| 61 | LCAESIGEVY IKSTETGQFL AMDTDGLLYG SQTPNEECLF LERLEENHYN TYISKKHAEK |
| 121 | HWFVGLKKNG RSKLGPRTHF GQKAILFLPL PVSSD |
| SEQ ID NO: 7 | |
| fibroblast growth factor 2 [Homo sapiens] | |
| NCBI Reference Sequence: NP_001997.5 | |
| 1 | MVGVGGGDVE DVTPRPGGCQ ISGRGARGCN GIPGAAAWEA ALPRRRPRRH PSVNPRSRAA |
| 61 | GSPRTRGRRT EERPSGSRLG DRGRGRALPG GRLGGRGRGR APERVGGRGR GRGTAAPRAA |
| 121 | PAARGSRPGP AGTMAAGSIT TLPALPEDGG SGAFPPGHFK DPKRLYCKNG GFFLRIHPDG |
| 181 | RVDGVREKSD PHIKLQLQAE ERGVVSIKGV CANRYLAMKE DGRLLASKCV TDECFFFERL |
| 241 | ESNNYNTYRS RKYTSWYVAL KRTGQYKLGS KTGPGQKAIL FLPMSAKS |
| SEQ ID NO: 8 | |
| fibroblast growth factor 2 (basic) [Homo sapiens] | |
| GenBank: EAX05222.1 | |
| 1 | MAAGSITTLP ALPEDGGSGA FPPGHFKDPK RLYCKNGGFF LRIHPDGRVD GVREKSDPHI |
| 61 | KLQLQAEERG VVSIKGVCAN RYLAMKEDGR LLASKCVTDE CFFFERLESN NYNTYRSRKY |
| 121 | TSWYVALKRT GQYKLGSKTG PGQKAILFLP MSAKS |
| SEQ ID NO: 9 | |
| fibroblast growth factor 2 [Mus musculus] | |
| GenBank: AAK53871.1 | |
| 1 | MAASGITSLP ALPEDGGAAF PPGHFKDPKR LYCKNGGFFL RIHPDGRVDG VREKSDPHVK |
| 61 | LQLQAEERGV VSIKGVCANR YLAMKEDGRL LASKCVTEEC FFFERLESNN YNTYRSRKYS |
| 121 | SWYVALKRTG QYKLGSKTGP GQKAILFLPM SAKS |
| SEQ ID NO: 10 | |
| fibroblast growth factor 2 [Rattus norvegicus] | |
| UniProtKB/Swiss-Prot: P13109.1 | |
| 1 | MAAGSITSLP ALPEDGGGAF PPGHFKDPKR LYCKNGGFFL RIHPDGRVDG VREKSDPHVK |
| 61 | LQLQAEERGV VSIKGVCANR YLAMKEDGRL LASKCVTEEC FFFERLESNN YNTYRSRKYS |
| 121 | SWYVALKRTG QYKLGSKTGP GQKAILFLPM SAKS |
| SEQ ID NO: 11 | |
| fibroblast growth factor 2 precursor [Bos taurus] | |
| Accession: NP_776481.2 | |
| 1 | MAAGSITTLP SLPEDGGSGA FPPGHFKDPK RLYCKNGGFF LRIHPDGRVD GVREKSDPHI |
| 61 | KLQLQAEERG VVSIKGVCAN RYLAMKEDGR LLASKCVTDE CFFFERLESN NYNTYRSRKY |
| 121 | SSWYVALKRT GQYKLGPKTG PGQKAILFLP MSAKS |
| SEQ ID NO: 12 | |
| fibroblast growth factor 3 precursor [Homo sapiens] | |
| NCBI Reference Sequence: NP_005238.1 | |
| 1 | MGLIWLLLLS LLEPGWPAAG PGARLRRDAG GRGGVYEHLG GAPRRRKLYC ATKYHLQLHP |
| 61 | SGRVNGSLEN SAYSILEITA VEVGIVAIRG LFSGRYLAMN KRGRLYASEH YSAECEFVER |
| 121 | IHELGYNTYA SRLYRTVSST PGARRQPSAE RLWYVSVNGK GRPRRGFKTR RTQKSSLFLP |
| 181 | RVLDHRDHEM VRQLQSGLPR PPGKGVQPRR RRQKQSPDNL EPSHVQASRL GSQLEASAH |
| SEQ ID NO: 13 | |
| Fibroblast growth factor 3 [Mus musculus] | |
| GenBank: AAI17062.1 | |
| 1 | MGLIWLLLLS LLEPSWPTTG PGTRLRRDAG GRGGVYEHLG GAPRRRKLYC ATKYHLQLHP |
| 61 | SGRVNGSLEN SAYSILEITA VEVGVVAIKG LFSGRYLAMN KRGRLYASDH YNAECEFVER |
| 121 | IHELGYNTYA SRLYRTGSSG PGAQRQPGAQ RPWYVSVNGK GRPRRGFKTR RTQKSSLFLP |
| 181 | RVLGHKDHEM VRLLQSSQPR APGEGSQPRQ RRQKKQSPSD HGKMETLSTR |
| 231 | ATPSTQLHTG GLAVA |
| SEQ ID NO: 14 | |
| FGF3 [Rattus norvegicus] | |
| GenBank: BAB84564.1 | |
| 1 | MGLIWLLLLS LLEPGWPATG PGTRLRRDAG GRGGVYEHLG GAPRRRKLYC ATKYHLQLHP |
| 61 | SGRVNGSLEN SAYSILEITA VEVGVVAIKG LFSGRYLAMN KRGRLYASEH YNAECEFVER |
| 121 | IHELGYNTYA SRLYRTGPSG PGARRQPGAQ RPWYVSVNGK GRPRRGFKTR RTQKSSLFLP |
| 181 | RVLGHKDHEM VRLLQSGQPQ APGEGSQPRQ RRQKKQSPGD HGKMEHLPTK |
| 231 | ATTSAQLDTG GLAMA |
| SEQ ID NO: 15 | |
| PREDICTED: FIBROBLAST GROWTH FACTOR 3 [BOS TAURUS] | |
| NCBI REFERENCE SEQUENCE: XP_002699485.1 | |
| 1 | MDLIWLLLLS LLEPGWPAAG PVARPRRDAG GRGGVYEHLG GAPRRRKLYC ATKYHLQLHP |
| 61 | SGRVNGSLEN SAYSILEITA VEVGVVAIKG LFSGRYLAMN KRGRLYASES YNAECEFVER |
| 121 | IHELGYNTYA SRLYRTAPSG RGARRQPSAE RLWYVSVNGK GRPRRGFKTR RTQKSSLFLP |
| 181 | RVLDRKDHEM VRLLLGTAGL RGGQARPPPP GRAASMRQRR RRQQRRPRDR DRGGRA |
| SEQ ID NO: 16 | |
| fibroblast growth factor 4 precursor [Homo sapiens] | |
| NCBI Reference Sequence: NP_001998.1 | |
| 1 | MSGPGTAAVA LLPAVLLALL APWAGRGGAA APTAPNGTLE AELERRWESL VALSLARLPV |
| 61 | AAQPKEAAVQ SGAGDYLLGI KRLRRLYCNV GIGFHLQALP DGRIGGAHAD TRDSLLELSP |
| 121 | VERGVVSIFG VASRFFVAMS SKGKLYGSPF FTDECTFKEI LLPNNYNAYE SYKYPGMFIA |
| 181 | LSKNGKTKKG NRVSPTMKVT HFLPRL |
| SEQ ID NO: 17 | |
| Fibroblast growth factor 4 [Mus musculus] | |
| GenBank: AAI04313.1 | |
| 1 | MAKRGPTTGT LLPRVLLALV VALADRGTAA PNGTRHAELG HGWDGLVARS LARLPVAAQP |
| 61 | PQAAVRSGAG DYLLGLKRLR RLYCNVGIGF HLQVLPDGRI GGVHADTRDS LLELSPVQRG |
| 121 | VVSIFGVASR FFVAMSSRGK LFGVPFFTDE CKFKEILLPN NYNAYESYAY PGMFMALSKN |
| 181 | GRTKKGNRVS PTMKVTHFLP RL |
| SEQ ID NO: 18 | |
| fibroblast growth factor 4 precursor [Rattus norvegicus] | |
| NCBI Reference Sequence: NP_446261.1 | |
| 1 | MAKRGPTTGT LLPGVLLALV VALADRGTAA PNGTRHAELG HGWDGLVARS LARLPVAAQP |
| 61 | PHAAVRSGAG DYLLGLKRLR RLYCNVGIGF HLQVLPDGRI GGVPRGHEGQ QRGVVSIFGV |
| 121 | ASRFFVAMSS RGKLFGVPFF TDECKFKEIL LPNNYNAYES YAYPGMFMAL SKNGRTKKGN |
| 181 | RVSPTMKVTH FLPRL |
| SEQ ID NO: 19 | |
| fibroblast growth factor 4 [Bos taurus] | |
| GenBank: BAL04177.1 | |
| 1 | MAGPGAAAAA LLPAVLLAVL APWAGRGGAA APTAPNGTLE AELERRWESL VARSLARLPV |
| 61 | AAQPKEAAVQ SGAGDYLLGI KRLRRLYCNV GIGFHLQVLP DGRIGGVHAD TSDSLLELSP |
| 121 | VERGVVSIFG VASRFFVAMS SRGRLYGSPF FTDECRFREI LLPNNYNAYE CDRHPGMFIA |
| 181 | LSKNGKAKKG NRVSPTMKVT HFLPRL |
| SEQ ID NO: 20 | |
| Fibroblast growth factor 5 [Homo sapiens] | |
| GenBank: AAH74858.1 | |
| 1 | MSLSFLLLLF FSHLILSAWA HGEKRLAPKG QPGPAATDRN PRGSSSRQSS SSAMSSSSAS |
| 61 | SSPAASLGSQ GSGLEQSSFQ WSPSGRRTGS LYCRVGIGFH LQIYPDGKVN GSHEANMLSV |
| 121 | LEIFAVSQGI VGIRGVFSNK FLAMSKKGKL HASAKFTDDC KFRERFQENS YNTYASAIHR |
| 181 | TEKTGREWYV ALNKRGKAKR GCSPRVKPQH ISTHFLPRFK QSEQPELSFT VTVPEKKKPP |
| 241 | SPIKPKIPLS APRKNTNSVK YRLKFRFG |
| SEQ ID NO: 21 | |
| Fibroblast growth factor 5 [Mus musculus] | |
| GenBank: AAH71227.1 | |
| 1 | MSLSLLFLIF CSHLIHSAWA HGEKRLTPEG QPAPPRNPGD SSGSRGRSSA TFSSSSASSP |
| 61 | VAASPGSQGS GSEHSSFQWS PSGRRTGSLY CRVGIGFHLQ IYPDGKVNGS HEASVLSILE |
| 121 | IFAVSQGIVG IRGVFSNKFL AMSKKGKLHA SAKFTDDCKF RERFQENSYN TYASAIHRTE |
| 181 | KTGREWYVAL NKRGKAKRGC SPRVKPQHVS THFLPRFKQS EQPELSFTVT VPEKKKPPVK |
| 241 | PKVPLSQPRR SPSPVKYRLK FRFG |
| SEQ ID NO: 22 | |
| fibroblast growth factor 5 [Rattus norvegicus] | |
| GenBank: EDL99619.1 | |
| 1 | MSLSLLFLIF CSHLILSAPA QGEKRLTPEG QPAPPRNPGD SSGSRGRSSA TFASSSASSP |
| 61 | VAASPGSQGS GSEHSSFQWS PSGRRTGSLY CRVGIGFHLQ IYPDGKVNGS HEASVLSILE |
| 121 | IFAVSQGIVG IRGVFSNKFL AMSKKGKLHA SAKFTDDCKF RERFQENSYN TYASAIHRTE |
| 181 | KTGREWYVAL NKRGKAKRGC SPRVKPQHVS THFLPRFKQS EQPELSFTVT VPEKKKPPSP |
| 241 | VKPKVPLSPP RRSPSPVKYR LKFRFG |
| SEQ ID NO: 23 | |
| fibroblast growth factor 5 [Bos taurus] | |
| GenBank: ABK34274.1 | |
| 1 | MSLSFLLLLF LSHLILSAWA QGEKRLAPKG QPGPAATERN PGGASSRRSS SSTATSSSSP |
| 61 | ASSSSAASRG GPGSSLEQSS FQWSPSGRRT GSLYCRVGIG FHLQIYPDGK VNGSHEANML |
| 121 | SILEIFAVSQ GIVGIRGVFS NKFLAMSKKG KLHASAKFTD DCKFRERFQE NSYNTYASAI |
| 181 | HRTEKTGREW YVALNKRGKA KRGCSPRVKP QHVSTHFLPR FKQLEQPELS FTVTVPEKKK |
| 241 | PPNPVKPKVP LSAPRRSPNT VKYRLKFRFG |
| SEQ ID NO: 24 | |
| Fibroblast growth factor 6 [Homo sapiens] | |
| GenBank: AAI21099.1 | |
| 1 | MALGQKLFIT MSRGAGRLQG TLWALVFLGI LVGMVVPSPA GTRANNTLLD SRGWGTLLSR |
| 61 | SRAGLAGEIA GVNWESGYLV GIKRQRRLYC NVGIGFHLQV LPDGRISGTH EENPYSLLEI |
| 121 | STVERGVVSL FGVRSALFVA MNSKGRLYAT PSFQEECKFR ETLLPNNYNA YESDLYQGTY |
| 181 | IALSKYGRVK RGSKVSPIMT VTHFLPRI |
| SEQ ID NO: 25 | |
| fibroblast growth factor 6 precursor [Mus musculus] | |
| NCBI Reference Sequence: NP_034334.1 | |
| 1 | MALGQRLFIT MSRGAGRVQG TLQALVFLGV LVGMVVPSPA GARANGTLLD SRGWGTLLSR |
| 61 | SRAGLAGEIS GVNWESGYLV GIKRQRRLYC NVGIGFHLQV PPDGRISGTH EENPYSLLEI |
| 121 | STVERGVVSL FGVKSALFIA MNSKGRLYTT PSFHDECKFR ETLLPNNYNA YESDLYRGTY |
| 181 | IALSKYGRVK RGSKVSPIMT VTHFLPRI |
| SEQ ID NO: 26 | |
| fibroblast growth factor 6 [Rattus norvegicus] | |
| NCBI Reference Sequence: NP_571983.1 | |
| 1 | MALGQRLFIT MSRGAGRVQG TLQALVFLGV LVGMVVPSPA GARANGTLLD SRGWGTLLSR |
| 61 | SRAGLAGEIS GVNWESGYLV GIKRQRRLYC NVGIGFHLQV PPDGRISGTH EENPYSLLEI |
| 121 | STVERGVVSL FGVKSALFIA MNSKGRLYTT PSFQDECKFR ETLLPNNYNA YESDLYRGTY |
| 181 | IALSKYGRVK RGSKVSPIMT VTHFLPRI |
| SEQ ID NO: 27 | |
| fibroblast growth factor 6 [Bos taurus] | |
| NCBI REFERENCE SEQUENCE: NP_001179329.1 | |
| 1 | MARGQTPLIT MSRGAGRPQG TLRALVFLGV LVGMVVPSPA GTRANGTLLA SRGWGTLLSR |
| 61 | SRAGLAGEIA GVNWESGYLV GIKRQRRLYC NVGIGFHLQV PPDGRISGTH EENPYSLLEI |
| 121 | STVERGVVSL FGVKSALFVA MNSKGKLYAT PSFQEECKFR ETLLPNNYNA YESDLYRGAY |
| 181 | IALSKYGRVK RGSKVSPTMT VTHFLPRI |
| SEQ ID NO: 28 | |
| fibroblast growth factor 7 precursor [Homo sapiens] | |
| NCBI Reference Sequence: NP_002000.1 | |
| 1 | MHKWILTWIL PTLLYRSCFH IICLVGTISL ACNDMTPEQM ATNVNCSSPE RHTRSYDYME |
| 61 | GGDIRVRRLF CRTQWYLRID KRGKVKGTQE MKNNYNIMEI RTVAVGIVAI KGVESEFYLA |
| 121 | MNKEGKLYAK KECNEDCNFK ELILENHYNT YASAKWTHNG GEMFVALNQK |
| 171 | GIPVRGKKTK KEQKTAHFLP MAIT |
| SEQ ID NO: 29 | |
| Fibroblast growth factor 7 [Mus musculus] | |
| GenBank: AAH52847.1 | |
| 1 | MRKWILTRIL PTLLYRSCFH LVCLVGTISL ACNDMSPEQT ATSVNCSSPE RHTRSYDYME |
| 61 | GGDIRVRRLF CRTQWYLRID KRGKVKGTQE MKNSYNIMEI RTVAVGIVAI KGVESEYYLA |
| 121 | MNKEGKLYAK KECNEDCNFK ELILENHYNT YASAKWTHSG GEMFVALNQK |
| 171 | GIPVKGKKTK KEQKTAHFLP MAIT |
| SEQ ID NO: 30 | |
| fibroblast growth factor 7 [Rattus norvegicus] | |
| GenBank: EDL80082.1 | |
| 1 | MRKWILTRIL PTPLYRSCFH LVCLVGTISL ACNDMSPEQT ATSVNCSSPE RHTRSYDYME |
| 61 | GGDIRVRRLF CRTQWYLRID KRGKVKGTQE MRNSYNIMEI RTVAVGIVAI KGVESEYYLA |
| 121 | MNKEGKLYAK KECNEDCNFK ELILENHYNT YASAKWTHSG GEMFVALNQK |
| 171 | GLPVKGKKTK KEQKTAHFLP MAIT |
| SEQ ID NO: 31 | |
| fibroblast growth factor 7 precursor [Bos taurus] | |
| NCBI Reference Sequence: NP_001180060.1 | |
| 1 | MRKWILTWIL PSLLYRSCFH IICLVGTISL ACNDMTPEQM ATNVNCSSPE RHTRSYDYME |
| 61 | GGDIRVRRLF CRTQWYLRID KRGKVKGTQE MKNNYNIMEI RTVAVGIVAI KGVESEYYLA |
| 121 | MNKEGKLYAK KECNEDCNFK ELILENHYNT YASAKWTHSG GEMFVALNQK |
| 171 | GVPVRGKKTK KEQKTAHFLP MAIT |
| SEQ ID NO: 32 | |
| fibroblast growth factor 8 precursor [Homo sapiens] | |
| GenBank: AAC50784.1 | |
| 1 | MGSPRSALSC LLLHLLVLCL QAQEGPGRGP ALGRELASLF RAGREPQGVS QQHVREQSLV |
| 61 | TDQLSRRLIR TYQLYSRTSG KHVQVLANKR INAMAEDGDP FAKLIVETDT FGSRVRVRGA |
| 121 | ETGLYICMNK KGKLIAKSNG KGKDCVFTEI VLENNYTALQ NAKYEGWYMA |
| 171 | FTRKGRPRKG SKTRQHQREV HFMKRLPRGH HTTEQSLRFE FLNYPPFTRS LRGSQRTWAP |
| 221 | EPR |
| SEQ ID NO: 33 | |
| Fgf8 protein [Mus musculus] | |
| GenBank: AAH48734.1 | |
| 1 | MGSPRSALSC LLLHLLVLCL QAQEGPGGGP ALGREPTSLL RAGREPQGVS QQVTVQSSPN |
| 61 | FTQHVREQSL VTDQLSRRLI RTYQLYSRTS GKHVQVLANK RINAMAEDGD PFAKLIVETD |
| 121 | TFGSRVRVRG AETGLYICMN KKGKLIAKSN GKGKDCVFTE IVLENNYTAL |
| 171 | QNAKYEGWYM AFTRKGRPRK GSKTRQHQRE VHFMKRLPRG HHTTEQSLRF |
| 221 | EFLNYPPFTR SLRGSQRTWA PEPR |
| SEQ ID NO: 34 | |
| FGF8 [Rattus norvegicus] | |
| GenBank: BAB84359.1 | |
| 1 | MGSPRSALSC LLLHLLVLCL QAQHVREQSL VTDQLSRRLI RTYQLYSRTS GKHVQVLANK |
| 61 | RINAMAEDGD PFAKLIVETD TFGSRVRVRG AETGLYICMN KKGKLIAKSN GKGKDCVFTE |
| 121 | IVLENNYTAL QNAKYEGWYM AFTRKGRPRK GSKTRQHQRE VHFMKRLPRG |
| 171 | HHTTEQSLRF EFLNYPPFTR SLRGSQRTWA PEPR |
| SEQ ID NO: 35 | |
| fibroblast growth factor 8 precursor [Bos taurus] | |
| NCBI Reference Sequence: NP_001193607.1 | |
| 1 | MGSPRSALSC LLLHLLVLCL QAQEGPGGGP ALGRELASLF RAGRESQGVS QQVTVQSSPN |
| 61 | FTQHVREQSL VTDQLSRRLI RTYQLYSRTS GKHVQVLANK RINAMAEDGD PFAKLIVETD |
| 121 | TFGSRVRVRG AETGLYICMN KKGKLIAKSN GKGKDCVFTE IVLENNYTAL |
| 171 | QNAKYEGWYM AFTRKGRPRK GSKTRQHQRE VHFMKRLPRG HHTTEQSLRF |
| 221 | EFLNYPPFTR SLRGSQRTWA PEPR |
| SEQ ID NO: 36 | |
| fibroblast growth factor 9 precursor [Homo sapiens] | |
| NCBI Reference Sequence: NP_002001.1 | |
| 1 | MAPLGEVGNY FGVQDAVPFG NVPVLPVDSP VLLSDHLGQS EAGGLPRGPA VTDLDHLKGI |
| 61 | LRRRQLYCRT GFHLEIFPNG TIQGTRKDHS RFGILEFISI AVGLVSIRGV DSGLYLGMNE |
| 121 | KGELYGSEKL TQECVFREQF EENWYNTYSS NLYKHVDTGR RYYVALNKDG |
| 171 | TPREGTRTKR HQKFTHFLPR PVDPDKVPEL YKDILSQS |
| SEQ ID NO: 37 | |
| fibroblast growth factor 9 [Mus musculus] | |
| GenBank: ADL60500.1 | |
| 1 | MAPLGEVGSY FGVQDAVPFG NVPVLPVDSP VLLSDHLGQS EAGGLPRGPA VTDLDHLKGI |
| 61 | LRRRQLYCRT GFHLEIFPNG TIQGTRKDHS RFGILEFISI AVGLVSIRGV DSGLYLGMNE |
| 121 | KGELYGSEKL TQECVFREQF EENWYNTYSS NLYKHVDTGR RCYVALNKDG |
| 171 | TPREGTRTKR HQKFTHFLPR PVDPDKVPEL YKDILSQS |
| SEQ ID NO: 38 | |
| fibroblast growth factor 9 precursor [Rattus norvegicus] | |
| NCBI Reference Sequence: NP_037084.1 | |
| 1 | MAPLGEVGSY FGVQDAVPFG NVPVLPVDSP VLLSDHLGQS EAGGLPRGPA VTDLDHLKGI |
| 61 | LRRRQLYCRT GFHLEIFPNG TIQGTRKDHS RFGILEFISI AVGLVSIRGV DSGLYLGMNE |
| 121 | KGELYGSEKL TQECVFREQF EENWYNTYSS NLYKHVDTGR RYYVALNKDG |
| 171 | TPREGTRTKR HQKFTHFLPR PVDPDKVPEL YKDILSQS |
| SEQ ID NO: 39 | |
| fibroblast growth factor 9 [Bos taurus] | |
| GenBank: ACG75898.1 | |
| 1 | MAPLGEVGNY FGVQDAVPFG NGPVLPVDSP VLLSDHLGQS EAGGLPRGPA VTDLDHLKGI |
| 61 | LRRRQLYCRT GFHLEIFPNG TIQGTRKDHS RFGILEFISI AVGLVSIRGV DSGLYLGMNE |
| 121 | KGELYGSEKL TQECVFREQF EENWYNTYSS NLYKHVDTGR RFYVALNKDG |
| 171 | TPREGTRTKR HQKFTHFLPR PVDPDKVPEL YKDILSQS |
| SEQ ID NO: 40 | |
| FGF10 [Homo sapiens] | |
| GenBank: CAG46466.1 | |
| 1 | MWKWILTHCA SAFPHLPGCC CCCFLLLFLV SSVPVTCQAL GQDMVSPEAT NSSSSSFSSP |
| 61 | SSAGRHVRSY NHLQGDVRWR KLFSFTKYFL KIEKNGKVSG TKKENCPYSI LEITSVEIGV |
| 121 | VAVKAINSNY YLAMNKKGKL YGSKEFNNDC KLKERIEENG YNTYASFNWQ |
| 171 | HNGRQMYVAL NGKGAPRRGQ KTRRKNTSAH FLPMVVHS |
| SEQ ID NO: 41 | |
| fibroblast growth factor 10 [Mus musculus] | |
| GenBank: EDL18354.1 | |
| 1 | MWKWILTHCA SAFPHLPGCC CCFLLLFLVS SFPVTCQALG QDMVSQEATN CSSSSSSFSS |
| 61 | PSSAGRHVRS YNHLQGDVRW RRLFSFTKYF LTIEKNGKVS GTKNEDCPYS VLEITSVEIG |
| 121 | VVAVKAINSN YYLAMNKKGK LYGSKEFNND CKLKERIEEN GYNTYASFNW |
| 171 | QHNGRQMYVA LNGKGAPRRG QKTRRKNTSA HFLPMTIQT |
| SEQ ID NO: 42 | |
| fibroblast growth factor 10 [Rattus norvegicus] | |
| GenBank: EDM10410.1 | |
| 1 | MWKWILTHCA SAFPHLPGCC CCFLLLFLVS SVPVTCQALG QDMVSPEATN SSSSSSSSSS |
| 61 | SSSFSSPSSA GRHVRSYNHL QGDVRWRKLF SFTKYFLKIE KNGKVSGTKK ENCPYSILEI |
| 121 | TSVEIGVVAV KAINSNYYLA MNKKGKLYGS KEFNNDCKLK ERIEENGYNT |
| 171 | YASFNWQHNG RQMYVALNGK GAPRRGQKTR RKNTSAHFLP MVVHS |
| SEQ ID NO: 43 | |
| fibroblast growth factor 10 precursor [Bos taurus] | |
| NCBI Reference Sequence: NP_001193255.1 | |
| 1 | MWKWILTHCA SAFPHLSGCC CCFLLLFLVS SVPVTCQALD QDMVSPGATN SSSSSSSSSS |
| 61 | SSVSLPSSAG RHVRSYNHLQ GDVRWRKLFS FTKYFLKIEN GKVSGTKKEN CPYSILEITS |
| 121 | VEIGVVAVKA INSNYYLAMN KKGKLYGSKE FNNDCKLKER IEENGYNTYA |
| 171 | SFNWQHNGRQ MYVALNGKGA PRRGQKTRRK NTSAHFLPMV VHS |
| SEQ ID NO: 44 | |
| Fibroblast growth factor 11 [Homo sapiens] | |
| GenBank: AAI08266.1 | |
| 1 | MAALASSLIR QKREVREPGG SRPVSAQRRV CPRGTKSLCQ KQLLILLSKV RLCGGRPARP |
| 61 | DRGPEPQLKG IVTKLFCRQG FYLQANPDGS IQGTPEDTSS FTHFNLIPVG LRVVTIQSAK |
| 121 | LGHYMAMNAE GLLYSSPHFT AECRFKECVF ENYYVLYASA LYRQRRSGRA |
| 171 | WYLGLDKEGQ VMKGNRVKKT KAAAHFLPKL LEVAMYQEPS LHSVPEASPS SPPAP |
| SEQ ID NO: 45 | |
| Fgf11 protein [Mus musculus] | |
| GenBank: AAH66859.1 | |
| 1 | MAALASSLIR QKREVREPGG SRPVSAQRRV CPRGTKSLCQ KQLLILLSKV RLCGGRPTRQ |
| 61 | DRGPEPQLKG IVTKLFCRQG FYLQANPDGS IQGTPEDTSS FTHFNLIPVG LRVVTIQSAK |
| 121 | LGHYMAMNAE GLLYSSRRSG RAWYLGLDKE GRVMKGNRVK KTKAAAHFVP |
| 171 | KLLEVAMYRE PSLHSVPETS PSSPPAH |
| SEQ ID NO: 46 | |
| FGF11 [Rattus norvegicus] | |
| GenBank: BAB84565.1 | |
| 1 | MAALASSLIR QKREVREPGG SRPVSAQRRV CPRGTKSLCQ KQLLILLSKV RLCGGRPTRQ |
| 61 | DRGPEPQLKG IVTKLFCRQG FYLQANPDGS IQGTPEDTSS FTHFNLIPVG LRVVTIQSAK |
| 121 | LGHYMAMNAE GLLYSSPHFT AECRFKECVF ENYYVLYASA LYRQRRSGRA |
| 171 | WYLGLDKEGR VMKGNRVKKT KAAAHFVPKL LEVAVYREPS LHSVPETSPS SPPAH |
| SEQ ID NO: 47 | |
| fibroblast growth factor 11 [Bos taurus] | |
| NCBI Reference Sequence: NP_001179868.1 | |
| 1 | MAALASSLIR QKREVREPGG SRPVSAQRRV CPRGTKSLCQ KQLLILLSKV RLCGGRPART |
| 61 | DRGPEPQLKG IVTKLFCRQG FYLQANPDGS IQGTPEDTSS FTHFNLIPVG LRVVTIQSAK |
| 121 | LGHYMAMNAE GLLYSSPHFT AECRFKECVF ENYYVLYASA LYRQRRSGRA |
| 171 | WYLGLDKEGR VMKGNRVKKT KAAAHFVPKL LEVAMYREPS LHSVPETSPS SPPAP |
| SEQ ID NO: 48 | |
| Fibroblast growth factor 12 [Homo sapiens] | |
| GenBank: AAH22524.1 | |
| 1 | MESKEPQLKG IVTRLFSQQG YFLQMHPDGT IDGTKDENSD YTLFNLIPVG LRVVAIQGVK |
| 61 | ASLYVAMNGE GYLYSSDVFT PECKFKESVF ENYYVIYSST LYRQQESGRA WFLGLNKEGQ |
| 121 | IMKGNRVKKT KPSSHFVPKP IEVCMYREQS LHEIGEKQGR SRKSSGTPTM NGGKVVNQDS |
| 181 | T |
| SEQ ID NO: 49 | |
| Fibroblast growth factor 12 [Mus musculus] | |
| GenBank: AAH30485.1 | |
| 1 | MESKEPQLKG IVTRLFSQQG YFLQMHPDGT IDGTKDENSD YTLFNLIPVG LRVVAIQGVK |
| 61 | ASLYVAMNGE GYLYSSDVFT PECKFKESVF ENYYVIYSST LYRQQESGRA WFLGLNKEGQ |
| 121 | IMKGNRVKKT KPSSHFVPKP IEVCMYREPS LHEIGEKQGR SRKSSGTPTM NGGKVVNQDS |
| 181 | T |
| SEQ ID NO: 50 | |
| fibroblast growth factor12 [Rattus norvegicus] | |
| GenBank: BAB84568.1 | |
| 1 | MAAAIASSLI RQKRQARESN SDRVSASKRR SSPSKDGRSL CERHVLGVFS KVRFCSGRKR |
| 61 | PVRRRPEPQL KGIVTRLFSQ QGYFLQMHPD GTIDGTKDEN SDYTLFNLIP VGLRVVAIQG |
| 121 | VKASLYVAMN GEGYLYSSDV FTPECKFKES VFENYYVIYS STLYRQQESG RAWFLGLNKE |
| 181 | GQIMKGNRVK KTKPSSHFVP KPIEVCMYRE PSLHEIGEKQ GRSRXSSGTP TMNGGKVVNQ |
| 241 | DST |
| SEQ ID NO: 51 | |
| Fibroblast growth factor 12 [Bos taurus] | |
| GenBank: AAI18170.1 | |
| 1 | MESKEPQLKG IVTRLFSQQG YFLQMHPDGT IDGTKDENSD YTLFNLIPVG LRVVAIQGVK |
| 61 | ASLYVAMNGE GYLYSSDVFT PECKFKESVF ENYYVIYSST LYRQQESGRA WFLGLNKEGQ |
| 121 | IMKGNRVKKT KPSSHFVPKP IEVCMYREPS LHEIGEKQGR SRKSSGTPTM NGGKVVNQDS |
| 181 | T |
| SEQ ID NO: 52 | |
| Fibroblast growth factor 13 [Homo sapiens] | |
| GenBank: AAH12347.1 | |
| 1 | MAAAIASSLI RQKRQARERE KSNACKCVSS PSKGKTSCDK NKLNVFSRVK LFGSKKRRRR |
| 61 | RPEPQLKGIV TKLYSRQGYH LQLQADGTID GTKDEDSTYT LFNLIPVGLR VVAIQGVQTK |
| 121 | LYLAMNSEGY LYTSELFTPE CKFKESVFEN YYVTYSSMIY RQQQSGRGWY LGLNKEGEIM |
| 181 | KGNHVKKNKP AAHFLPKPLK VAMYKEPSLH DLTEFSRSGS GTPTKSRSVS GVLNGGKSMS |
| 241 | HNEST |
| SEQ ID NO: 53 | |
| Fibroblast growth factor 13 [Mus musculus] | |
| GenBank: AAH18238.1 | |
| 1 | MAAAIASSLI RQKRQARERE KSNACKCVSS PSKGKTSCDK NKLNVFSRVK LFGSKKRRRR |
| 61 | RPEPQLKGIV TKLYSRQGYH LQLQADGTID GTKDEDSTYT LFNLIPVGLR VVAIQGVQTK |
| 121 | LYLAMNSEGY LYTSEHFTPE CKFKESVFEN YYVTYSSMIY RQQQSGRGWY LGLNKEGEIM |
| 181 | KGNHVKKNKP AAHFLPKPLK VAMYKEPSLH DLTEFSRSGS GTPTKSRSVS GVLNGGKSMS |
| 241 | HNEST |
| SEQ ID NO: 54 | |
| Fibroblast growth factor 13 [Rattus norvegicus] | |
| UniProtKB/Swiss-Prot: Q9ERW3.2 | |
| 1 | MAAAIASSLI RQKRQARERE KSNACKCVSS PSKGKTSCDK NKLNVFSRVK LFGSKKRRRR |
| 61 | RPEPQLKGIV TKLYSRQGYH LQLQADGTID GTKDEDSTYT LFNLIPVGLR VVAIQGVQTK |
| 121 | LYLAMNSEGY LYTSEHFTPE CKFKESVFEN YYVTYSSMIY RQQQSGRGWY LGLNKEGEIM |
| 181 | KGNHVKKNKP AAHFLPKPLK VAMYKEPSLH DLTEFSRSGS GTPTKSRSVS GVLNGGKSMS |
| 241 | HNEST |
| SEQ ID NO: 55 | |
| FGF13 protein [Bos taurus] | |
| GenBank: AAI46027.1 | |
| 1 | MAAAIASSLI RQKRQARERE KSNACKCVSS PSKGKTSCDK NKLNVFSRVK LFGSKKRRRR |
| 61 | RPEPQLKGIV TKLYSRQGYH LQLQADGTID GTKDEDSTYT LFNLIPVGLR VVAIQGVQTK |
| 121 | LYLAMNSEGY LYTSEHFTPE CKFKESVFEN YYVTYSSMIY RQQQSGRGWY LGLNKEGEIM |
| 181 | KGNHVKKNKP AAHFLPKPLK VAMYKEPSLH DLTEFSRSGS GTPTKSRSVS GVLNGGKSMS |
| 241 | HNEST |
| SEQ ID NO: 56 | |
| Fibroblast growth factor 14 [Homo sapiens] | |
| GenBank: AAI00923.1 | |
| 1 | MVKPVPLFRR TDFKLLLCNH KDLFFLRVSK LLDCFSPKSM WFLWNIFSKG THMLQCLCGK |
| 61 | SLKKNKNPTD PQLKGIVTRL YCRQGYYLQM HPDGALDGTK DDSTNSTLFN LIPVGLRVVA |
| 121 | IQGVKTGLYI AMNGEGYLYP SELFTPECKF KESVFENYYV IYSSMLYRQQ ESGRAWFLGL |
| 181 | NKEGQAMKGN RVKKTKPAAH FLPKPLEVAM YREPSLHDVG ETVPKPGVTP |
| 231 | SKSTSASAIM NGGKPVNKSK TT |
| SEQ ID NO: 57 | |
| fibroblast growth factor 14 [Mus musculus] | |
| GenBank: EDL02968.1 | |
| 1 | MAAAIASGLI RQKRQAREQH WDRPSASRRR SSPSKNRGLC NGNLVDIFSK VRIFGLKKRR |
| 61 | LRRQDPQLKG IVTRLYCRQG YYLQMHPDGA LDGTKDDSTN STLFNLIPVG LRVVAIQGVK |
| 121 | TGLYIAMNGE GYLYPSELFT PECKFKESVF ENYYVIYSSM LYRQQESGRA WFLGLNKEGQ |
| 181 | VMKGNRVKKT KPAAHFLPKP LEVAMYREPS LHDVGETVPK AGVTPSKSTS |
| 231 | ASAIMNGGKP VNKCKTT |
| SEQ ID NO: 58 | |
| fibroblast growth factor14 [Rattus norvegicus] | |
| GenBank: BAB84580.1 | |
| 1 | MAAAIASGLI RQKRQAREQH WDRPSASRRR SSPSKNRGLC NGNLVDIFSK VRIFGLKKRR |
| 61 | LRRQDPQLKG IVTRLYCRQG YYLQMHPDGA LDGTKDDSTN STLFNLIPVG LRVVAIQGVK |
| 121 | TGLYIAMNGE GYLYPSELFT PECKFKESVF ENYYVIYSSM LYRQQESGRA WFLGLNKEGQ |
| 181 | VMKGNRVKKT KPAAHFLPKP LEVAMYREPS LHDVGETVPK AGVTPSKSTS |
| 231 | ASAIMNGGKP VNKCKTT |
| SEQ ID NO: 59 | |
| fibroblast growth factor 14 [Bos taurus] | |
| NCBI Reference Sequence: NP_001193761.1 | |
| 1 | MVKPVPLFRR TDFKLLLCNH KDLFFLRVSK LLDCFSPKSM WFLWNIFSKG THMLQCLCGK |
| 61 | SLKKNKNPTD PQLKGIVTRL YCRQGYYLQM HPDGALDGTK EDSTNSTLFN LIPVGLRVVA |
| 121 | IQGVKTGLYV AMNGEGYLYP SELFTPECKF KESVFENYYV IYSSMLYRQQ ESGRAWFLGL |
| 181 | NKEGQVMKGN RVKKTKPAAH FLPKPLEVAM YREPSLHDVG ETVPKAGVTP |
| 231 | SKSTSASAIM NGGKPVNKSK TT |
| SEQ ID NO: 60 | |
| FGF15 [MUS MUSCULUS] | |
| GENBANK: AAO13811.1 | |
| 1 | MARKWNGRAV ARALVLATLW LAVSGRPLAQ QSQSVSDEDP LFLYGWGKIT RLQYLYSAGP |
| 61 | YVSNCFLRIR SDGSVDCEED QNERNLLEFR AVALKTIAIK DVSSVRYLCM SADGKIYGLI |
| 121 | RYSEEDCTFR EEMDCLGYNQ YRSMKHHLHI IFIQAKPREQ LQDQKPSNFI PVFHRSFFET |
| 181 | GDQLRSKMFS LPLESDSMDP FRMVEDVDHL VKSPSFQK |
| SEQ ID NO: 61 | |
| fibroblast growth factor 15 [Rattus norvegicus] | |
| GenBank: BAB84298.1 | |
| 1 | MARKWSGRIV ARALVLATLW LAVSGRPLVQ QSQSVSDEGP LFLYGWGKIT RLQYLYSAGP |
| 61 | YVSNCFLRIR SDGSVDCEED QNERNLLEFR AVALKTIAIK DVSSVRYLCM SADGKIYGLI |
| 121 | RYSEEDCTFR EEMDCLGYNQ YRSMKHHLHI IFIKAKPREQ LQGQKPSNFI PIFHRSFFES |
| 181 | TDQLRSKMFS LPLESDSMDP FRMVEDVDHL VKSPSFQK |
| SEQ ID NO: 62 | |
| fibroblast growth factor 19 precursor [Homo sapiens] | |
| NCBI Reference Sequence: NP_005108.1 | |
| 1 | MRSGCVVVHV WILAGLWLAV AGRPLAFSDA GPHVHYGWGD PIRLRHLYTS GPHGLSSCFL |
| 61 | RIRADGVVDC ARGQSAHSLL EIKAVALRTV AIKGVHSVRY LCMGADGKMQ GLLQYSEEDC |
| 121 | AFEEEIRPDG YNVYRSEKHR LPVSLSSAKQ RQLYKNRGFL PLSHFLPMLP MVPEEPEDLR |
| 181 | GHLESDMFSS PLETDSMDPF GLVTGLEAVR SPSFEK |
| SEQ ID NO: 63 | |
| PREDICTED: fibroblast growth factor 19 [Bos taurus] | |
| NCBI Reference Sequence: XP_599739.3 | |
| 1 | MNATEDISES SSALRSVITV RCSPVPARRA PRELHAQPLE KLSGTQGQHR RRKTQQKQRS |
| 61 | LPALRALERT AAGRARPIPG LKRHLALARA TLLFLREPRS RLAPSRGTKA SGPPPSLPHP |
| 121 | HRQICAQSSE PEGGAMRSAP SRCAVARALV LAGLWLAAAG RPLAFSDAGP |
| 171 | HVHYGWGESV RLRHLYTAGP QGLYSCFLRI HSDGAVDCAQ VQSAHSLMEI |
| 231 | RAVALSTVAI KGERSVLYLC MDADGKMQGL TQYSAEDCAF EEEIRPDGYN |
| 281 | VYWSRKHHLP VSLSSSRQRQ LFKSRGFLPL SHFLPMLSTI PAEPEDLQEP LKPDFFLPLK |
| 241 | TDSMDPFGLA TKLGSVKSPS FYN |
| SEQ ID NO: 64 | |
| fibroblast growth factor 20 [Homo sapiens] | |
| NCBI Reference Sequence: NP_062825.1 | |
| 1 | MAPLAEVGGF LGGLEGLGQQ VGSHFLLPPA GERPPLLGER RSAAERSARG GPGAAQLAHL |
| 61 | HGILRRRQLY CRTGFHLQIL PDGSVQGTRQ DHSLFGILEF ISVAVGLVSI RGVDSGLYLG |
| 121 | MNDKGELYGS EKLTSECIFR EQFEENWYNT YSSNIYKHGD TGRRYFVALN |
| 171 | KDGTPRDGAR SKRHQKFTHF LPRPVDPERV PELYKDLLMY T |
| SEQ ID NO: 65 | |
| fibroblast growth factor 20 [Mus musculus] | |
| GenBank: BAB16406.1 | |
| 1 | MAPLTEVGAF LGGLEGLGQQ VGSHFLLPPA GERPPLLGER RGALERGARG GPGSVELAHL |
| 61 | HGILRRRQLY CRTGFHLQIL PDGTVQGTRQ DHSLFGILEF ISVAVGLVSI RGVDSGLYLG |
| 121 | MNDKGELYGS EKLTSECIFR EQFEENWYNT YSSNIYKHGN TGRRYFVALN |
| | KDGTPRDGAR SKRRQKFTHF LPRPVDPERV PELYKDLLMY TG |
| SEQ ID NO: 66 | |
| fibroblast growth factor 20 [Rattus norvegicus] | |
| GenBank: EDL78810.1 | |
| 1 | MAPLTEVGAF LGGLEGLGQQ VGSHFLLPPA GERPPLLGER RGALERGARG GPGSVELAHL |
| 61 | HGILRRRQLY CRTGFHLQIL PDGSVQGTRQ DHSLFGILEF ISVAVGLVSI RGVDSGLYLG |
| 121 | MNGKGELYGS EKLTSECIFR EQFEENWYNT YSSNIYKHGD TGRRYFVALN |
| 171 | KDGTPRDGAR SKRHQKFTHF LPRPVDPERV PELYKDLLVY TG |
| SEQ ID NO: 67 | |
| fibroblast growth factor 20 [Bos taurus] | |
| NCBI Reference Sequence: NP_001179094.1 | |
| 1 | MAPLAEVGGF LGGLEGLGQQ VGSHFLLPPA GERPPLLGER RSAAERGARG GPGAAELAHL |
| 61 | HGFLRRRQLY CRTGFHLQIL PDGSVQGTRQ DHSLFGILEF ISVAVGLVSI RGVDSGLYLG |
| 121 | MNDKGELYGS EKLTSECIFR EQFEENWYNT YSSNIYKHGD TGRRYFVALN |
| 171 | KDGTPRDGAR SKRHQKFTHF LPRPVDPERV PELYKDLLMY S |
| SEQ ID NO: 68 | |
| FGF21 [Homo sapiens] | |
| GenBank: AAQ89444.1 | |
| 1 | MDSDETGFEH SGLWVSVLAG LLGACQAHPI PDSSPLLQFG GQVRQRYLYT DDAQQTEAHL |
| 61 | EIREDGTVGG AADQSPESLL QLKALKPGVI QILGVKTSRF LCQRPDGALY GSLHFDPEAC |
| 121 | SFRELLLEDG YNVYQSEAHG LPLHLPGNKS PHRDPAPRGP ARFLPLPGLP PALPEPPGIL |
| 181 | APQPPDVGSS DPLSMVGPSQ GRSPSYAS |
| SEQ ID NO: 69 | |
| fibroblast growth factor 21 [Rattus norvegicus] | |
| GenBank: BAB84299.1 | |
| 1 | MDWMKSRVGA PGLWVCLLLP VFLLGVCEAY PISDSSPLLQ FGGQVRQRYL YTDDDQDTEA |
| 61 | HLEIREDGTV VGTAHRSPES LLELKALKPG VIQILGVKAS RFLCQQPDGT LYGSPHFDPE |
| 121 | ACSFRELLLK DGYNVYQSEA HGLPLRLPQK DSQDPATRGP VRFLPMPGLP HEPQEQPGVL |
| 181 | PPEPPDVGSS DPLSMVEPLQ GRSPSYAS |
| SEQ ID NO: 70 | |
| fibroblast growth factor 21 [Bos taurus] | |
| UniProtKB - ElBDA6 | |
| 1 | MGWDEAKFKH LGLWVPVLAV LLLGTCRAHP IPDSSPLLQF GGQVRQRYLY |
| 51 | TDDAQETEAH LEIRADGTVV GAARQSPESL LELKALKPGV IQILGVKTSR |
| 101 | FLCQGPDGKL YGSLHFDPKA CSFRELLLED GYNVYQSETL GLPLRLPPQR |
| 151 | SSNRDPAPRG PARFLPLPGL PAAPPDPPGI LAPEPPDVGS SDPLSMVGPS |
| 201 | YGRSPSYTS |
| SEQ ID NO: 71 | |
| FGF22 [Homo sapiens] | |
| GenBank: AAQ89955.1 | |
| 1 | MRRRLWLGLA WLLLARAPDA AGTPSASRGP RSYPHLEGDV RWRRLFSSTH FFLRVDPGGR |
| 61 | VQGTRWRHGQ DSILEIRSVH VGVVVIKAVS SGFYVAMNRR GRLYGSRLYT VDCRFRERIE |
| 121 | ENGHNTYASQ RWRRRGQPMF LALDRRGGPR PGGRTRRYHL SAHFLPVLVS |
| SEQ ID NO: 72 | |
| Fgf22 protein [Mus musculus] | |
| GenBank: AAI19136.1 | |
| 1 | MRSRLWLGLA WLLLARAPGA PGGYPHLEGD VRWRRLFSST HFFLRVDLGG |
| 51 | RVQGTRWRHG QDSIVEIRSV RVGTVVIKAV YSGFYVAMNR RGRLYGSVPG AHRGERLQHI |
| 120 | RLATLEAPRP THVPGT |
| SEQ ID NO: 73 | |
| fibroblast growth factor 22 [Rattus norvegicus] | |
| GenBank: BAB84300.1 | |
| 1 | MRRRLWLGLA WLLLARAPGA PGGYPHLEGD VRWRRLFSST HFFLRVDPGG |
| 51 | RVQGTRWRHG QDSIVEIRSV RVGTVVIKAV YSGFYVAMNR RGRLYGSRVY SVDCRFRERI |
| 111 | EENGYNTYAS RRWRHHGRPM FLALDSQGIP RQGRRTRRHQ LSTHFLPVLV SS |
| SEQ ID NO: 74 | |
| fibroblast growth factor 22 precursor [Bos taurus] | |
| NCBI Reference Sequence: NP_001192790.1 | |
| 1 | MRGRLWLGLV WLLLARAPGT AGTLNTPRRP RSYPHLEGDV RWRRLFSSTH FFLLVDPSGR |
| 61 | VQGTRWRDNP DSVLEIRSIR VGVVVLKAVH SGFYVAMNRL GRLYGSRFCA AHCRFRERIE |
| 121 | ENGYNTYASV RWRHQGRPMF LALDGRGAPR LGGRTQRHHP STLFLPVLVS |
| SEQ ID NO: 75 | |
| FGF23 [Homo sapiens] | |
| GenBank: AAG09917.1 | |
| 1 | MLGARLRLWV CALCSVCSMS VLRAYPNASP LLGSSWGGLI HLYTATARNS YHLQIHKNGH |
| 61 | VDGAPHQTIY SALMIRSEDA GFVVITGVMS RRYLCMDFRG NIFGSHYFDP ENCRFQHQTL |
| 121 | ENGYDVYHSP QYHFLVSLGR AKRAFLPGMN PPPYSQFLSR RNEIPLIHFN TPIPRRHTRS |
| 181 | AEDDSERDPL NVLKPRARMT PAPASCSQEL PSAEDNSPMA SDPLGVVRGG |
| 231 | RVNTHAGGTG PEGCRPFAKF I |
| SEQ ID NO: 76 | |
| FGF23 [Mus musculus] | |
| GenBank: AAG09916.1 | |
| 1 | MLGTCLRLLV GVLCTVCSLG TARAYPDTSP LLGSNWGSLT HLYTATARTS YHLQIHRDGH |
| 61 | VDGTPHQTIY SALMITSEDA GSVVITGAMT RRFLCMDLHG NIFGSLHFSP ENCKFRQWTL |
| 121 | ENGYDVYLSQ KHHYLVSLGR AKRIFQPGTN PPPFSQFLAR RNEVPLLHFY TVRPRRHTRS |
| 181 | AEDPPERDPL NVLKPRPRAT PVPVSCSREL PSAEEGGPAA SDPLGVLRRG RGDARGGAGG |
| 241 | ADRCRPFPRF V |
| SEQ ID NO: 77 | |
| fibroblast growth factor 23 [Rattus norvegicus] | |
| GenBank: BAB84108.1 | |
| 1 | MLGACLRLLV GALCTVCSLG TARAYSDTSP LLGSNWGSLT HLYTATARNS YHLQIHRDGH |
| 61 | VDGTPHQTIY SALMITSEDA GSVVIIGAMT RRFLCMDLRG NIFGSYHFSP ENCRFRQWTL |
| 121 | ENGYDVYLSP KHHYLVSLGR SKRIFQPGTN PPPFSQFLAR RNEVPLLHFY TARPRRHTRS |
| 181 | AEDPPERDPL NVLKPRPRAT PIPVSCSREL PSAEEGGPAA SDPLGVLRRG RGDARRGAGG |
| 241 | TDRCRPFPRF V |
| SEQ ID NO: 78 | |
| PREDICTED: fibroblast growth factor 23 [Bos taurus] | |
| NCBI Reference Sequence: XP_003582326.1 | |
| 1 | MLGARLGLWV CTLSCVVQAY PNSSPLLGSS WGGLTHLYTA TARNSYHLQI HGDGHVDGSP |
| 61 | QQTVYSALMI RSEDAGFVVI TGVMSRRYLC MDFTGNIFGS HHFSPESCRF RQRTLENGYD |
| 121 | VYHSPQHRFL VSLGRAKRAF LPGTNPPPYA QFLSRRNEIP LPHFAATARP RRHTRSAHDS |
| 181 | GDPLSVLKPR ARATPVPAAC SQELPSAEDS GPAASDPLGV LRGHRLDVRA GSAGAERCRP |
| 241 | FPGFA |

In some embodiments, the FGF1 polypeptide comprises amino acids 1-155 of amino acids in SEQ ID NO: 1. In another embodiment, the FGF1 polypeptide derived from human FGF1 comprises amino acids 25-155 of amino acids in SEQ ID NO: 1.

In some embodiments, the FGF1 polypeptide is a mammalian homolog of human FGF1 or a functional fragment thereof and reduces or normalizes elevated blood glucose when administered to the brain. In some embodiments, the FGF1 polypeptide has an amino acid sequence at least 85%, at least 90%, at least 95%, at least 97% or at least 99% identical to the amino acid sequence of SEQ ID NO:1 and reduces or normalizes elevated blood glucose when administered to the brain. In some embodiments, the FGF1 polypeptide has an amino acid sequence that has at least 85%, at least 90%, at least 95%, at least 97% or at least 99% amino acid sequence homology to amino acid sequence of SEQ ID NO: 1 and reduces or normalizes elevated blood glucose when administered to the brain. Percent (%) amino acid sequence identity for a given polypeptide sequence relative to a reference sequence is defined as the percentage of identical amino acid residues identified after aligning the two sequences and introducing gaps if necessary, to achieve the maximum percent sequence identity, and not considering any conservative substitutions as part of the sequence identity. Percent (%) amino acid sequence homology for a given polypeptide sequence relative to a reference sequence is defined as the percentage of identical or strongly similar amino acid residues identified after aligning the two sequences and introducing gaps if necessary, to achieve the maximum percent homology. Non identities of amino acid sequences include conservative substitutions, deletions or additions that do not affect the blood sugar reducing or normalizing activity of FGF1. Strongly similar amino acids can include, for example, conservative substitutions known in the art. Percent identity and/or homology can be calculated using alignment methods known in the art, for instance alignment of the sequences can be conducted using publicly available software software such as BLAST, Align, ClustalW2. Those skilled in the art can determine the appropriate parameters for alignment, but the default parameters for BLAST are specifically contemplated.

The FGF-1 polypeptide can be recombinant, purified, isolated, naturally occurring or synthetically produced. The term “recombinant” when used in reference to a nucleic acid, protein, cell or a vector indicates that the nucleic acid, protein, vector or cell containing them have been modified by introduction of a heterologous nucleic acid or protein or the alteration of a native nucleic acid or a protein, or that the cell is derived from a cell so modified. The term “heterologous” (meaning ‘derived from a different organism’) refers to the fact that often the transferred protein was initially derived from a different cell type or a different species from the recipient. Typically the protein itself is not transferred, but instead the genetic material coding for the protein (often the complementary DNA or cDNA) is added to the recipient cell. Methods of generating and isolating recombinant polypeptides are known to those skilled in the art and can be performed using routine techniques in the field of recombinant genetics and protein expression. For standard recombinant methods, see Sambrook et al, Molecular Cloning: A Laboratory Manual, Cold Spring Harbor Laboratory Press, N Y (1989); Deutscher, Methods in Enzymology 182:83-9 (1990); Scopes, Protein Purification: Principles and Practice, Springer-Verlag, NY (1982).

### Biological Activity of FGF1 Polypeptides

In addition to the required activity of reducing or normalizing abnormally elevated blood sugar when administered to the brain, FGF1 polypeptides as described herein can also have biological activities of FGF1 including, for example, binding to heparin and heparan sulfate, and binding to one or more FGF receptors.

### Binding to FGF Receptors.

FGF family members exert their activities by binding to one or more of the four FGF receptors (FGFRs) FGFR1-FGFR4. The receptor binding specificity of individual FGFs and their isoforms is distinct. FGF1 binds to all four receptors. FGFRs consist of three extracellular immunoglobulin domains (D1-D3), a single-pass transmembrane domain and a cytoplasmic tyrosine kinase domain. A hallmark of FGFRs is the presence of an acidic, serine-rich sequence in the linker between D1 and D2, termed the acid box. The D2-D3 fragment of the FGFR ectodomain is necessary and sufficient for ligand binding and specificity, whereas the D1 domain and the acid box are proposed to have a role in receptor autoinhibition. Several FGFR isoforms exist, as exon skipping removes the D1 domain and/or acid box in FGFR1-FGFR3. Alternative splicing in the second half of the D3 domain of FGFR1-3 yields b (FGFR1b-3b) and c (FGFR1c-3c) isoforms that have distinct FGF binding specificities and are predominantly epithelial and mesenchymal, respectively. Each FGF binds to either epithelial or mesenchymal FGFRs, with the exception of FGF1, which activates both splice isoforms. Alternative splicing in Ig domain III dramatically changes the specificity of the FGFR for certain FGFs. This splicing event is tissue-specific and is essential for directional FGF signaling across epithelial-mesenchymal boundaries (such as in the developing limb bud). The heparin-binding domain is a stretch of 18 conserved amino acid residues and is essential for receptor activity and by itself has the capacity to interact with heparin. FGF-FGFR binding specificity is regulated both by primary sequence differences between the 18 FGFs and the 4 main FGFRs (FGFR1, FGFR2, FGFR3, and FGFR4). Structural studies of FGF1, FGF2, FGF8 and FGF10 with their cognate FGFRs show that sequence diversity at FGF N-termini, variation in β1 strand length and the alternatively spliced regions in D3 dictate their binding specificities.

In some embodiments, the FGF1 polypeptide of the methods described herein can bind one or more FGFRs or ligand-binding fragment(s) thereof. The polypeptide and coding nucleic acid sequences of FGFRs of human origin and those for a number of animals are publicly available, e.g., from the NCBI website. Examples include but are not limited to;

| SEQ ID NO: 79 | |
|---|---|
| FGFR1 protein [Homo sapiens] | |
| GenBank: AAH15035.1 | |
| 1 | MWSWKCLLFW AVLVTATLCT ARPSPTLPEQ AQPWGAPVEV ESFLVHPGDL LQLRCRLRDD |
| 61 | VQSINWLRDG VQLAESNRTR ITGEEVEVQD SVPADSGLYA CVTSSPSGSD TTYFSVNVSD |
| 121 | ALPSSEDDDD DDDSSSEEKE TDNTKPNRMP VAPYWTSPEK MEKKLHAVPA |
| 171 | AKTVKFKCPS SGTPNPTLRW LKNGKEFKPD HRIGGYKVRY ATWSIIMDSV |
| 221 | VPSDKGNYTC IVENEYGSIN HTYQLDVVER SPHRPILQAG LPANKTVALG SNVEFMCKVY |
| 281 | SDPQPHIQWL KHIEVNGSKI GPDNLPYVQI LKTAGVNTTD KEMEVLHLRN VSFEDAGEYT |
| 341 | CLAGNSIGLS HHSAWLTVLE ALEERPAVMT SPLYLEIIIY CTGAFLISCM VGSVIVYKMK |
| 401 | SGTKKSDFHS QMAVHKLAKS IPLRRQVSAD SSASMNSGVL LVRPSRLSSS GTPMLAGVSE |
| 461 | YELPEDPRWE LPRDRLVLGK PLGEGCFGQV VLAEAIGLDK DKPNRVTKVA |
| 511 | VKMLKSDATE KDLSDLISEM EMMKMIGKHK NIINLLGACT QDGPLYVIVE |
| 561 | YASKGNLREY LQARRPPGLE YCYNPSHNPE EQLSSKDLVS CAYQVARGME |
| 611 | YLASKKCIHR DLAARNVLVT EDNVMKIADF GLARDIHHID YYKKTTNGRL |
| 661 | PVKWMAPEAL FDRIYTHQSD VWSFGVLLWE IFTLGGSPYP GVPVEELFKL LKEGHRMDKP |
| 721 | SNCTNELYMM MRDCWHAVPS QRPTFKQLVE DLDRIVALTS NQEYLDLSMP |
| 771 | LDQYSPSFPD TRSSTCSSGE DSVFSHEPLP EEPCLPRHPA QLANGGLKRR |
| SEQ ID NO: 80 | |
| Fibroblast growth factor receptor 1 [Mus musculus] | |
| GENBANK: AAH10200.1 | |
| 1 | MWGWKCLLFW AVLVTATLCT ARPAPTLPEQ AQPWGVPVEV ESLLVHPGDL LQLRCRLRDD |
| 61 | VQSINWLRDG VQLVESNRTR ITGEEVEVRD SIPADSGLYA CVTSSPSGSD TTYFSVNVSD |
| 121 | ALPSSEDDDD DDDSSSEEKE TDNTKPNPVA PYWTSPEKME KKLHAVPAAK TVKFKCPSSG |
| 181 | TPNPTLRWLK NGKEFKPDHR IGGYKVRYAT WSIIMDSVVP SDKGNYTCIV ENEYGSINHT |
| 241 | YQLDVVERSP HRPILQAGLP ANKTVALGSN VEFMCKVYSD PQPHIQWLKH IEVNGSKIGP |
| 301 | DNLPYVQILK TAGVNTTDKE MEVLHLRNVS FEDAGEYTCL AGNSIGLSHH SAWLTVLEAL |
| 361 | EERPAVMTSP LYLEIIIYCT GAFLISCMLG SVIIYKMKSG TKKSDFHSQM AVHKLAKSIP |
| 421 | LRRQVTVSAD SSASMNSGVL LVRPSRLSSS GTPMLAGVSE YELPEDPRWE LPRDRLVLGK |
| 481 | PLGEGCFGQV VLAEAIGLDK DKPNRVTKVA VKMLKSDATE KDLSDLISEM |
| 531 | EMMKMIGKHK NIINLLGACT QDGPLYVIVE YASKGNLREY LQARRPPGLE YCYNPSHNPE |
| 591 | EQLSSKDLVS CAYQVARGME YLASKKCIHR DLAARNVLVT EDNVMKIADF |
| 641 | GLARDIHHID YYKKTTNGRL PVKWMAPEAL FDRIYTHQSD VWSFGVLLWE IFTLGGSPYP |
| 701 | GVPVEELFKL LKEGHRMDKP SNCTNELYMM MRDCWHAVPS QRPTFKQLVE |
| 751 | DLDRIVALTS NQEYLDLSIP LDQYSPSFPD TRSSTCSSGE DSVFSHEPLP EEPCLPRHPT |
| 811 | QLANSGLKRR |
| SEQ ID NO: 81 | |
| fibroblast growth factor receptor 1 precursor [Rattus norvegicus] | |
| NCBI Reference Sequence: NP_077060.1 | |
| 1 | MWGWRGLLFW AVLVTATLCT ARPAPTLPEQ AQPWGVPVEV ESLLVHPGDL LQLRCRLRDD |
| 61 | VQSINWLRDG VQLAESNRTR ITGEEVEVRD SIPADSGLYA CVTNSPSGSD TTYFSVNVSD |
| 121 | ALPSSEDDDD DDDSSSEEKE TDNTKPNRRP VAPYWTSPEK MEKKLHAVPA AKTVKFKCPS |
| 181 | SGTPSPTLRW LKNGKEFKPD HRIGGYKVRY ATWSIIMDSV VPSDKGNYTC IVENEYGSIN |
| 241 | HTYQLDVVER SPHRPILQAG LPANKTVALG SNVEFMCKVY SDPQPHIQWL KHIEVNGSKI |
| 301 | GPDNLPYDQI LKTAGVNTTD KEMEVLHLRN VSFEDAGEYT CLAGNSIGLS HHSAWLTVLE |
| 361 | ALEERPAVMT SPLYLEIIIY CTGAFLISCM VGSVIIYKMK SGTKKSDFHS QMAVHKLAKS |
| 421 | IPLRRQVTVS ADSSASMNSG VLLVRPSRLS SSGTPMLAGV SEYELPEDPR WELPRDRLVL |
| 481 | GKPLGEGCFG QVVLAEAIGL DKDKPNRVTK VAVKMLKSDA TEKDLSDLIS |
| 531 | EMEMMKMIGK HKNIINLLGA CTQDGPLYVI VEYASKGNLR EYLQARRPPG |
| 581 | LEYCYNPSHN PEEQLSSKDL VSCAYQVARG MEYLASKKCI HRDLAARNVL |
| 631 | VTEDNVMKIA DFGLARDIHH IDYYKKTTNG RLPVKWMAPE ALFDRIYTHQ |
| 681 | SDVWSFGVLL WEIFTLGGSP NPGVPVEELF KLLKEGHRMD KPSNCTNELY |
| 731 | MMMRDCWNAV PSQRPTFKQL VEDLDRIVAL TSNQEYLDLS MPLDQDSPSF PDTRSSTCSS |
| 791 | GEDSVFSHEP FPEEPCLPRH PTQLANGGLN RR |
| SEQ ID NO: 82 | |
| FGFR1 protein [Bos taurus] | |
| GenBank: AAI34638.2 | |
| 1 | MWSRKCLLFW AVLVTATLCT AKPAPTLPEQ AQPWGAPVEV ESLLVHPGDL LQLRCRLRDD |
| 61 | VQSINWLRDG VQLADSNRTR ITGEEVEVRG SVPADSGLYA CVTSSPSGSD TTYFSVNVSD |
| 121 | ALPSSEDDDD DDDSSSEEKE TDNTKPNPVA PYWTSPEKME KKLHAVPAAK TVKFKCPSSG |
| 181 | TPNPTLRWLK NGKEFKPDHR IGGYKVRYAT WSIIMDSVVP SDKGNYTCIV ENEYGSINHT |
| 241 | YQLDVVERSP HRPILQAGLP ANKTVALGSN VEFMCKVYSD PQPHIQWLKH IEVNGSKIGP |
| 301 | DNLPYVQILK TAGVNTTDKE MEVLHLRNVS FEDAGEYTCL AGNSIGLSHH SAWLTVLEAL |
| 361 | EERPAVMTSP LYLEIIIYCT GAFLISCMVG SVIIYKMKSG TKKSDFHSQM AVHKLAKSIP |
| 421 | LRRQVTVSAD SSASMNSGVL LVRPSRLSSS GTPMLAGVSE YELPEDPRWE LPRDRLVLGK |
| 481 | PLGEGCFGQV VLAEAIGLDK DRPNRVTKVA VKMLKSDATE KDLSDLISEM |
| 531 | EMMKMIGKHK NIINLLGACT QDGPLYVIVE YASKGNLREY LQARRPPGLE YCYNPSHHPE |
| 591 | EQLSSKDLVS CAYQVARGME YLASKKCIHR DLAARNVLVT EDNVMKIADF |
| 641 | GLARDIHHID YYKKTTNGRL PVKWMAPEAL FDRIYTHQSD VWSFGVLLWE IFTLGGSPYP |
| 701 | GVPVEELFKL LKEGHRMDKP SNCTNELYMM MRDCWHAVPS QRPTFKQLVE |
| 751 | DLDRIVALTS NQEYLDLSMP LDQYSPSFPD TRSSTCSSGE DSVFSHEPLP EEPCLPRHPA |
| 811 | QLANGGLKRR |
| SEQ ID NO: 83 | |
| FGFR2 [Homo sapiens] | |
| GenBank: CAA96492.1 | |
| 1 | MVSWGRFICL VVVTMATLSL ARPSFSLVED TTLEPEEPPT KYQISQPEVY VAAPGESLEV |
| 61 | RCLLKDAAVI SWTKDGVHLG PNNRTVLIGE YLQIKGATPR DSGLYACTAS RTVDSETWYF |
| 121 | MVNVTDAISS GDDEDDTDGA EDFVSENSNN KRAPYWTNTE KMEKRLHAVP |
| 171 | AANTVKFRCP AGGNPMPTMR WLKNGKEFKQ EHRIGGYKVR NQHWSLIMES |
| 221 | VVPSDKGNYT CVVENEYGSI NHTYHLDVVE RSPHRPILQA GLPANASTVV GGDVEFVCKV |
| 281 | YSDAQPHIQW IKHVEKNGSK YGPDGLPYLK VLKAAGVNTT DKEIEVLYIR NVTFEDAGEY |
| 341 | TCLAGNSIGI SFHSAWLTVL PAPGREKEIT ASPDYLEIAI YCIGVFLIAC MVVTVILCRM |
| 401 | KNTTKKPDFS SQPAVHKLTK RIPLRRQVTV SAESSSSMNS NTPLVRITTR LSSTADTPML |
| 461 | AGVSEYELPE DPKWEFPRDK LTLGKPLGEG CFGQVVMAEA VGIDKDKPKE |
| 511 | AVTVAVKMLK DDATEKDLSD LVSEMEMMKM IGKHKNIINL LGACTQDGPL |
| 561 | YVIVEYASKG NLREYLRARR PPGMEYSYDI NRVPEEQMTF KDLVSCTYQL |
| 611 | ARGMEYLASQ KCIHRDLAAR NVLVTENNVM KIADFGLARD INNIDYYKKT |
| 661 | TNGRLPVKWM APEALFDRVY THQSDVWSFG VLMWEIFTLG GSPYPGIPVE ELFKLLKEGH |
| 721 | RMDKPANCTN ELYMMMRDCW HAVPSQRPTF KQLVEDLDRI LTLTTNEEYL |
| 771 | DLSQPLEQYS PSYPDTRSSC SSGDDSVFSP DPMPYEPCLP QYPHINGSVK T |
| SEQ ID NO: 84 | |
| FGFR2 [Mus musculus] | |
| GenBank: ABL89211.1 | |
| 1 | MVSWGRFICL VLVTMATLSL ARPSFSLVED TTLEPEEPPT KYQISQPEAY VVAPGESLEL |
| 61 | QCMLKDAAVI SWTKDGVHLG PNNRTVLIGE YLQIKGATPR DSGLYACTAA RTVDSETWIF |
| 121 | MVNVTDAISS GDDEDDTDSS EDVVSENRSN QRAPYWTNTE KMEKRLHACP |
| 171 | AANTVKFRCP AGGNPTSTMR WLKNGKEFKQ EHRIGGYKVR NQHWSLIMES |
| 221 | VVPSDKGNYT CLVENEYGSI NHTYHLDVVE RSPHRPILQA GLPANASTVV |
| 226 | GGDVEFVCKV YSDAQPHIQW IKHVEKNGSK NGPDGLPYLK VLKAAGVNTT |
| 276 | DKEIEVLYIR NVTFEDAGEY TCLAGNSIGI SFHSAWLTVL PAPVREKEIT ASPDYLEIAI |
| 336 | YCIGVFLIAC MVVTVIFCRM KTTTKKPDFS SQPAVHKLTK RIPLRRQVTV SAESSSSMNS |
| 396 | NTPLVRITTR LSSTADTPML AGVSEYELPE DPKWEFPRDK LTLGKPLGEG CFGQVVMAEA |
| 456 | VGIDKDKPKE AVTVAVKMLK DDATEKDLSD LVSEMEMMKM IGKHKNIINL |
| 516 | LGACTQDGPL YVIVEYASKG NLREYLRARR PPGMEYSYDI NRVPEEQMTF |
| 566 | KDLVSCTYQL ARGMEYLASQ KCIHRDLAAR NVLVTENNVM KIADFGLARD |
| 616 | INNIDYYKKT TNGRLPVKWM APEALFDRVY THQSDVWSFG VLMWEIFTLG |
| 666 | GSPYPGIPVE ELFKLLKEGH RMDKPTNCTN ELYMMMRDCW HAVPSQRPTF |
| 716 | KQLVEDLDRI LTLTTNEEYL DLTQPLEQYS PSYPDTRSSC SSGDDSVFSP DPMPYEPCLP |
| 776 | QYPHINGSVK T |
| SEQ ID NO: 85 | |
| fibroblast growth factor receptor 2 isoform a [Rattus norvegicus] | |
| NCBI Reference Sequence: NP_036844.1 | |
| 1 | MGLPSTWRYG TGPGIGTVTM VSWGRFICLV LVTMATLSLA RPSFSLVEDT TLEPEEPPTK |
| 61 | YQISQPEACV VAPGESLELR CMLKDAAVIS WTKDGVHLGP NNRTVLIGEY LQIKGATPRD |
| 121 | SGLYACAAAR TVDSETLYFM VNVTDAISSG DDEDDTDSSE DFVSENRSNQ |
| 171 | RAPYWTNTEK MEKRLHAVPA ANTVKFRCPA GGNPTPTMRW LKNGKEFKQE |
| 221 | HRIGGYKVRN QHWSLIMESV VPSDKGNYTC LVENEYGSIN HTYHLDVVER SPHRPILQAG |
| 282 | LPANASTVVG GDVEFVCKVY SDAQPHIQWI KHVEKNGSKY GPDGLPYLKV |
| 331 | LKHSGINSSN AEVLALFNVT EMDAGEYICK VSNYIGQANQ SAWLTVLPKQ QAPVREKEIT |
| 391 | ASPDYLEIAI YCIGVFLIAC MVVTVIFCRM KTTTKKPDFS SQPAVHKLTK RIPLRRQVTV |
| 451 | SAESSSSMNS NTPLVRITTR LSSTADTPML AGVSEYELPE DPKWEFPRDK LTLGKPLGEG |
| 511 | CFGQVVMAEA VGIDKDRPKE AVTVAVKMLK DDATEKDLSD LVSEMEMMKM |
| 561 | IGKHKNIINL LGACTQDGPL YVIVEYASKG NLREYLRARR PPGMEYSYDI NRVPEEQMTF |
| 621 | KDLVSCTYQL ARGMEYLASQ KCIHRDLAAR NVLVTENNVM KIADFGLARD |
| 671 | INNIDYYKKT TNGRLPVKWM APEALFDRVY THQSDVWSFG VLMWEIFTLG |
| 721 | GSPYPGIPVE ELFKLLKEGH RMDKPTNCTN ELYMMMRDCW HAVPSQRPTF |
| 771 | KQLVEDLDRI LTLTTNEEYL DLTQPLEQYS PSYPDTRSSC SSGDDSVFSP DPMPYDPCLP |
| 831 | QYPHINGSVK T |
| SEQ ID NO: 86 | |
| FIBROBLAST GROWTH FACTOR RECEPTOR 2 [BOS TAURUS] | |
| NCBI REFERENCE SEQUENCE: NP_001192239.1 | |
| 1 | MGLTSTWRYG RGQGIGTVTM VSWGRFLCLV VVTMATLSLA RPSFNLVDDT TVEPEEPPTK |
| 61 | YQISQPEVYV AAPRESLELR CLLRDAAMIS WTKDGVHLGP NNRTVLIGEY LQIKGATPRD |
| 121 | SGLYACTAAR NVDSETVYFM VNVTDAISSG DDEDDADGSE DFVSENSNSK |
| 171 | RAPYWTNTEK MEKRLHAVPA ANTVKFRCPA GGNPTPTMRW LKNGKEFKQE |
| 221 | HRIGGYKVRN QHWSLIMESV VPSDKGNYTC VVENDYGSIN HTYHLDVVER |
| 271 | SPHRPILQAG LPANASTVVG GDVEFVCKVY SDAQPHIQWI KHVEKNGSKY |
| 321 | GPDGLPYLKV LKHSGINSSN AEVLALFNVT EADAGEYICK VSNYIGQANQ SAWLTVLPKQ |
| 381 | QAPVREKEIP ASPDYLEIAI YCIGVFFIAC MVVTVILCRM RNTTKKPDFS SQPAVHKLTK |
| 441 | RIPLRRQVSA ESSSSMNSNT PLVRITTRLS STADTPMLAG VSEYELPEDP KWEFPRDKLT |
| 501 | LGKPLGEGCF GQVVMAEAVG IDKEKPKEAV TVAVKMLKDD ATEKDLSDLV |
| 551 | SEMEMMKMIG KHKNIINLLG ACTQDGPLYV IVEYASKGNL REYLRARRPP |
| 601 | GMEYSYDINR VPEEQMAFKD LVSCTYQLAR GMEYLASQKC IHRDLAARNV |
| 651 | LVTENNVMKI ADFGLARDIN NIDYYKKTTN GRLPVKWMAP EALFDRVYTH |
| 701 | QSDVWSFGVL MWEIFTLGGS PYPGIPVEEL FKLLKEGHRM DKPANCTNEL |
| 751 | YMMMRDCWHA VPSQRPTFKQ LVEDLDRILT LTTNEEYLDL SQLLEQYSPS YPDTRSSCSS |
| 811 | GDDSVFSPDP MPYEPCLPQY PHRNGSVKT |
| SEQ ID NO: 87 | |
| FIBROBLAST GROWTH FACTOR RECEPTOR 3 PRECURSOR [HOMO SAPIENS] | |
| UNIPROTKB/SWISS-PROT: P22607.1 | |
| 1 | MGAPACALAL CVAVAIVAGA SSESLGTEQR VVGRAAEVPG PEPGQQEQLV FGSGDAVELS |
| 61 | CPPPGGGPMG PTVWVKDGTG LVPSERVLVG PQRLQVLNAS HEDSGAYSCR QRLTQRVLCH |
| 121 | FSVRVTDAPS SGDDEDGEDE AEDTGVDTGA PYWTRPERMD KKLLAVPAAN |
| 171 | TVRFRCPAAG NPTPSISWLK NGREFRGEHR IGGIKLRHQQ WSLVMESVVP SDRGNYTCVV |
| 231 | ENKFGSIRQT YTLDVLERSP HRPILQAGLP ANQTAVLGSD VEFHCKVYSD AQPHIQWLKH |
| 291 | VEVNGSKVGP DGTPYVTVLK TAGANTTDKE LEVLSLHNVT FEDAGEYTCL |
| 241 | AGNSIGFSHH SAWLVVLPAE EELVEADEAG SVYAGILSYG VGFFLFILVV AAVTLCRLRS |
| 301 | PPKKGLGSPT VHKISRFPLK RQVSLESNAS MSSNTPLVRI ARLSSGEGPT LANVSELELP |
| 461 | ADPKWELSRA RLTLGKPLGE GCFGQVVMAE AIGIDKDRAA KPVTVAVKML |
| 511 | KDDATDKDLS DLVSEMEMMK MIGKHKNIIN LLGACTQGGP LYVLVEYAAK |
| 561 | GNLREFLRAR RPPGLDYSFD TCKPPEEQLT FKDLVSCAYQ |
| 601 | VARGMEYLAS QKCIHRDLAA RNVLVTEDNV MKIADFGLAR DVHNLDYYKK |
| 651 | TTNGRLPVKW MAPEALFDRV YTHQSDVWSF GVLLWEIFTL GGSPYPGIPV EELFKLLKEG |
| 711 | HRMDKPANCT HDLYMIMREC WHAAPSQRPT FKQLVEDLDR VLTVTSTDEY |
| 761 | LDLSAPFEQY SPGGQDTPSS SSSGDDSVFA HDLLPPAPPS SGGSRT |
| SEQ ID NO: 88 | |
| FIBROBLAST GROWTH FACTOR RECEPTOR 3 [MUS MUSCULUS] | |
| GENBANK: AAH53056.1 | |
| 1 | MVVPACVLVF CVAVVAGATS EPPGPEQRVV RRAAEVPGPE PSQQEQVAFG SGDTVELSCH |
| 61 | PPGGAPTGPT VWAKDGTGLV ASHRILVGPQ RLQVLNASHE DAGVYSCQHR LTRRVLCHFS |
| 121 | VRVTDAPSSG DDEDGEDVAE DTGAPYWTRP ERMDKKLLAV PAANTVRFRC |
| 171 | PAAGNPTPSI SWLKNGKEFR GEHRIGGIKL RHQQWSLVME SVVPSDRGNY TCVVENKFGS |
| 231 | IRQTYTLDVL ERSPHRPILQ AGLPANQTAI LGSDVEFHCK VYSDAQPHIQ WLKHVEVNGS |
| 291 | KVGPDGTPYV TVLKTAGANT TDKELEVLSL HNVTFEDAGE YTCLAGNSIG |
| 341 | FSHHSAWLVV LPAEEELMET DEAGSVYAGV LSYGVVFFLF ILVVAAVILC RLRSPPKKGL |
| 401 | GSPTVHKVSR FPLKRQVSLE SNSSMNSNTP LVRIARLSSG EGPVLANVSE LELPADPKWE |
| 461 | LSRTRLTLGK PLGEGCFGQV VMAEAIGIDK DRTAKPVTVA VKMLKDDATD |
| 511 | KDLSDLVSEM EMMKMIGKHK NIINLLGACT QGGPLYVLVE YAAKGNLREF |
| 561 | LRARRPPGMD YSFDACRLPE EQLTCKDLVS CAYQVARGME |
| 601 | YLASQKCIHR DLAARNVLVT EDNVMKIADF GLARDVHNLD YYKKTTNGRL |
| 651 | PVKWMAPEAL FDRVYTHQSD VWSFGVLLWE IFTLGGSPYP GIPVEELFKL |
| 701 | LKEGHRMDKP ASCTHDLYMI MRECWHAVPS QRPTFKQLVE DLDRILTVTS |
| 751 | TDEYLDLSVP FEQYSPGGQD TPSSSSSGDD SVFTHDLLPP GPPSNGGPRT |
| SEQ ID NO: 89 | |
| FIBROBLAST GROWTH FACTOR RECEPTOR 3 [RATTUS NORVEGICUS] | |
| GENBANK: AAF97795.1 | |
| 1 | MVVPACVLVF CVAVVAGVTS EPPGPEQRVG RRAAEVPGPE PSQQEQVAFG SGDTVELSCH |
| 61 | PPGGAPTGPT LWAKDGVGLV ASHRILVGPQ RLQVLNATHE DAGVYSCQQR LTRRVLCHFS |
| 121 | VRVTDAPSSG DDEDGEDVAE DTGAPYWTRP ERMDKKLLAV PAANTVRFRC |
| 171 | PAAGNPTPSI PWLKNGKEFR GEHRIGGIKL RHQQWSLVME SVVPSDRGNY TCVVENKFGS |
| 231 | IRQTYTLDVL ERSPHRPILQ AGLPANQTAV LGSDVEFHCK VYSDAQPHIQ WLKHVEVNGS |
| 291 | KVGPDGTPYV TVLKTAGANT TDRELEVLSL HNVTFEDAGE YTCLAGNSIG |
| 341 | FSHHSAWLVV LPAEEELMEV DEAGSVYAGV LSYGVGFFLF ILVVAAVTLC RLRSPPKKGL |
| 401 | GSPTVHKVSR FPLKRQVSLE SNSSMNSNTP LVRIARLSSG EGPVLANVSE LELPADPKWE |
| 461 | LSRTRLTLGK PLGEGCFGQV VMAEAIGIDK DRTAKPVTVA VKMLKDDATD |
| 511 | KDLSDLVSEM EMMKMIGKHK NIINLLGACT QGGPLYVLVE YAAKGNLREF |
| 561 | LRARRPPGMD YSFDACRLPE EQLTCKDLVS CAYQVARGME YLASQKCIHR |
| 611 | DLAARNVLVT EDNVMKIADF GLARDVHNLD YYKKTTNGRL PVKWMAPEAL |
| 661 | FDRVYTHQSD VWSFGVLLWE IFTLGGSPYP GIPVEELFKL LKEGHRMDKP ANCTHDLYMI |
| 721 | MRECWHAVPS QRPTFKQLVE DLDRILTVTS TDEYLDLSVP FEQYSPGGQD TPSSSSSGDD |
| 781 | SVFTHDLLPP GPPSNGGPRT |
| SEQ ID NO: 90 | |
| FIBROBLAST GROWTH FACTOR RECEPTOR 3 [BOS TAURUS] | |
| GENBANK: BAB69587.1 | |
| 1 | MGAPARALAF CVAVAVMTGA ALGSPGVEPR VARRAAEVPG PEPSPQERAF GSGDTVELSC |
| 61 | RLPAGVPTEP TVWVKDGVGL APSDRVLVGP QRLQVLNASH EDAGAYSCRQ RLSQRLLCLF |
| 121 | SVRVTDAPSS GDDEGGDDEA EDTAGAPYWT RPERMDKKLL AVPAANTVRF |
| 171 | RCPAAGNPTP SITWLKNGKE FRGEHRIGGI KLRQQQWSLV MESVVPSDRG |
| 221 | NYTCVVENKF GRIQQTYTLD VLERSPHRPI LQAGLPANQT AVLGSDVEFH CKVYSDAQPH |
| 281 | IQWLKHVEVN GSKVGPDGTP YVTVLKTAGA NTTDKELEVL SLRNVTFEDA |
| 331 | GEYTCLAGNS IGFSHHSAWL VVLPAEEELV EAGEAGGVFA GVLSYGLGFL LFILAVAAVT |
| 391 | LYRLRSPPKK GLGSPAVHKV SRFPLKRQVS LESSSSMSSN TPLVRIARLS SGEGPTLANV |
| 451 | SELELPADPK WELSRARLTL GKPLGEGCFG QVVMAEAIGI DKDRAAKPVT |
| 501 | VAVKMLKDDA TDKDLSDLVS EMEMMKMIGK HKNIINLLGA CTQGGPLYVL |
| 551 | VEYAAKGNLR EYLRARRPPG TDYSFDTCRL PEEQLTFKDL VSCAYQVARG |
| 601 | MEYLASQKCI HRDLAARNVL VTEDNVMKIA DFGLARDVHN LDYYKKTTNG |
| 651 | RLPVKWMAPE ALFDRVYTHQ SDVWSFGVLL WEIFTLGGSP YPGIPVEELF |
| 701 | KLLKEGHRMD KPANCTHDLY MIMRECWHAA PSQRPTFKQL VEDLDRVLTV |
| 751 | TSTDEYLDLS VPFEQYSPGG QDTPSSGSSG DDSVFAHDLL PPAPSGSGGS RT |
| SEQ ID NO: 91 | |
| FIBROBLAST GROWTH FACTOR RECEPTOR 4 [HOMO SAPIENS] | |
| GENBANK: AAB59389.1 | |
| 1 | MRLLLALLGV LLSVPGPPVL SLEASEEVEL EPCLAPSLEQ QEQELTVALG QPVRLCCGRA |
| 61 | ERGGHWYKEG SRLAPAGRVR GWRGRLEIAS FLPEDAGRYL CLARGSMIVL QNLTLITGDS |
| 121 | LTSSNDDEDP KSHRDPSNRH SYPQQAPYWT HPQRMEKKLH AVPAGNTVKF |
| 171 | RCPAAGNPTP TIRWLKDGQA FHGENRIGGI RLRHQHWSLV MESVVPSDRG |
| 221 | TYTCLVENAV GSIRYNYLLD VLERSPHRPI LQAGLPANTT AVVGSDVELL CKVYSDAQPH |
| 281 | IQWLKHIVIN GSSFGADGFP YVQVLKTADI NSSEVEVLYL RNVSAEDAGE YTCLAGNSIG |
| 341 | LSYQSAWLTV LPEEDPTWTA AAPEARYTDI ILYASGSLAL AVLLLLAGLY RGQALHGRHP |
| 401 | RPPATVQKLS RFPLARQFSL ESGSSGKSSS SLVRGVRLSS SGPALLAGLV SLDLPLDPLW |
| 461 | EFPRDRLVLG KPLGEGCFGQ VVRAEAFGMD PARPDQASTV AVKMLKDNAS |
| 511 | DKDLADLVSE MEVMKLIGRH KNIINLLGVC TQEGPLYVIV ECAAKGNLRE FLRARRPPGP |
| 571 | DLSPDGPRSS EGPLSFPVLV SCAYQVARGM QYLESRKCIH RDLAARNVLV |
| 621 | TEDNVMKIAD FGLARGVHHI DYYKKTSNGR LPVKWMAPEA LFDRVYTHQS |
| 671 | DVWSFGILLW EIFTLGGSPY PGIPVEELFS LLREGHRMDR PPHCPPELYG |
| 721 | LMRECWHAAP SQRPTFKQLV EALDKVLLAV SEEYLDLRLT FGPYSPSGGD ASSTCSSSDS |
| 781 | VFSHDPLPLG SSSFPFGSGV QT |
| SEQ ID NO: 92 | |
| FIBROBLAST GROWTH FACTOR RECEPTOR 4 [MUS MUSCULUS] | |
| GENBANK: AAH33313.1 | |
| 1 | MWLLLALLSI FQGTPALSLE ASEEMEQEPC LAPILEQQEQ VLTVALGQPV RLCCGRTERG |
| 61 | RHWYKEGSRL ASAGRVRGWR GRLEIASFLP EDAGRYLCLA RGSMTVVHNL TLLMDDSLTS |
| 121 | ISNDEDPKTL SSSSSGHVYP QQAPYWTHPQ RMEKKLHAVP AGNTVKFRCP AAGNPMPTIH |
| 181 | WLKDGQAFHG ENRIGGIRLR HQHWSLVMES VVPSDRGTYT CLVENSLGSI RYSYLLDVLE |
| 241 | RSPHRPILQA GLPANTTAVV GSDVELLCKV YSDAQPHIQW LKHVVINGSS FGADGFPYVQ |
| 301 | VLKTTDINSS EVEVLYLRNV SAEDAGEYTC LAGNSIGLSY QSAWLTVLPE EDLTWTTATP |
| 361 | EARYTDIILY VSGSLVLLVL LLLAGVYHRQ VIRGHYSRQP VTIQKLSRFP LARQFSLESR |
| 421 | SSGKSSLSLV RGVRLSSSGP PLLTGLVNLD LPLDPLWEFP RDRLVLGKPL GEGCFGQVVR |
| 481 | AEAFGMDPSR PDQTSTVAVK MLKDNASDKD LADLVSEMEV MKLIGRHKNI |
| 531 | INLLGVCTQE GPLYVIVECA AKGNLREFLR ARRPPGPDLS PDGPRSSEGP LSFPALVSCA |
| 591 | YQVARGMQYL ESRKCIHRDL AARNVLVTED DVMKIADFGL ARGVHHIDYY |
| 641 | KKTSNGRLPV KWMAPEALFD RVYTHQSDVW SFGILLWEIF TLGGSPYPGI PVEELFSLLR |
| 701 | EGHRMERPPN CPSELYGLMR ECWHAVPSQR PTFKQLVEAL DKVLLAVSEE |
| 751 | YLDLRLTFGP FSPSNGDASS TCSSSDSVFS HDPLPLEPSP FPFSDSQTT |
| SEQ ID NO: 93 | |
| FIBROBLAST GROWTH FACTOR RECEPTOR 4 [RATTUS NORVEGICUS] | |
| GENBANK: AAI00261.1 | |
| 1 | MWLLLALLSI FQETPAFSLE ASEEMEQEPC PAPISEQQEQ VLTVALGQPV RLCCGRTERG |
| 61 | RHWYKEGSRL ASAGRVRGWR GRLEIASFLP EDAGRYLCLA RGSMTVVHNL TLIMDDSLPS |
| 121 | INNEDPKTLS SSSSGHSYLQ QAPYWTHPQR MEKKLHAVPA GNTVKFRCPA |
| 171 | AGNPMPTIHW LKNGQAFHGE NRIGGIRLRH QHWSLVMESV VPSDRGTYTC LVENSLGSIR |
| 231 | YSYLLDVLER SPHRPILQAG LPANTTAVVG SNVELLCKVY SDAQPHIQWL KHIVINGSSF |
| 291 | GADGFPYVQV LKTTDINSSE VEVLYLRNVS AEDAGEYTCL AGNSIGLSYQ SAWLTVLPAE |
| 351 | EEDLAWTTAT SEARYTDIIL YVSGSLALVL LLLLAGVYHR QAIHGHHSRQ PVTVQKLSRF |
| 411 | PLARQFSLES RSSGKSSLSL VRGVRLSSSG PPLLTGLVSL DLPLDPLWEF PRDRLVLGKP |
| 471 | LGEGCFGQVV RAEALGMDSS RPDQTSTVAV KMLKDNASDK DLADLISEME |
| 521 | MMKLIGRHKN IINLLGVCTQ EGPLYVIVEY AAKGNLREFL RARRPPGPDL SPDGPRSSEG |
| 581 | PLSFPALVSC AYQVARGMQY LESRKCIHRD LAARNVLVTE DDVMKIADFG |
| 631 | LARGVHHIDY YKKTSNGRLP VKWMAPEALF DRVYTHQSDV WSFGILLWEI |
| 681 | FTLGGSPYPG IPVEELFSLL REGHRMERPP NCPSELYGLM RECWHAAPSQ RPTFKQLVEA |
| 741 | LDKVLLAVSE EYLDLRLTFG PYSPNNGDAS STCSSSDSVF SHDPLPLEPS PFPFPEAQTT |
| SEQ ID NO: 94 | |
| FIBROBLAST GROWTH FACTOR RECEPTOR 4 PRECURSOR [BOS TAURUS] | |
| NCBI REFERENCE SEQUENCE: NP_001179513.1 | |
| 1 | MRLLLVLLGV LLGAPGAPAL SFEASEETEL EPCLAPSPEQ QEQELTVALG QPVRLCCGRA |
| 61 | ERSGHWYKEG SRLTPAGRVR GWRGRLEIAS FLPEDAGQYL CLSRGSLLLH NVTLVVDDSM |
| 121 | TSSNGDEDPK IHRGPLNGHV YPQQAPYWTH PQRMEKKLHA VPAGNTVKFR |
| 171 | CPAAGNPMPT IRWLKDGQDF HGEHRIGGIR LRHQHWSLVM ESVVPSDRGT |
| 221 | YTCLVENSLG SIRYSYLLDV LERSPHRPIL QAGLPANTTA VVGSDVELLC KVYSDAQPHI |
| 281 | QWLKHIVING SSFGADGFPY VQVLKTADIN SSEVEVLYLR NVSAEDAGEY TCLAGNSIGL |
| 341 | SYQSAWLTVL PEEDLTWTAT APEGRYTDII LYSSGSLALI VFLLLVGLYR RQTLLTRHHR |
| 401 | QPATVQKLSR FPLARQFSLE SGSSAKSSLS LVRGVRLSSS GPPLLAGLVS LDLPLDPLWE |
| 461 | FPRDRLVLGK PLGEGCFGQV VCAEAFGMDP TRPDQASTVA VKMLKDNASD |
| 511 | KDLADLVSEM EVMKLIGRHK NIINLLGVCT QEGPLYVIVE CAAKGNLREF LRARRPPGPD |
| 571 | LSPDGPRSSE GPLSFPALVS CAYQVARGMQ YLESRKCIHR DLAARNVLVT EDNVMKIADF |
| 631 | GLARGIHHID YYKKTSNGRL PVKWMAPEAL FDRVYTHQSD VWSFGILLWE IFTLGGSPYP |
| 691 | GIPVEELFSL LREGHRMDRP PHCPPELYGL MRECWHAAPS QRPTFKQLVE ALDKVLLAVS |
| 751 | EEYLDLRLTF GPYSPAGGDA SSTCSSSDSV |
| 781 | FSHDPLPLRP SSFSFPGVQT |

### Binding to Heparin or Heparan Sulfate.

In addition to FGFRs, the FGFs also bind to heparan sulfate proteoglycans (HPSGs) and their analog, heparin. Thus, in some embodiment, an FGF polypeptide as described herein can bind heparin and/or heparan sulfate proteoglycans. These interactions facilitate FGF-FGFR dimerization by simultaneously binding both FGF and FGFR, thereby promoting and stabilizing protein-protein contacts between ligand and receptor. The interaction also stabilizes FGFs against proteolysis and thermal denaturation, and heparan sulfate-bound FGF acts as a storage reservoir for FGFs. Heparan sulfate binding determines the radius of FGF diffusion by limiting its diffusion into interstitial spaces. The heparan sulfate glycosaminoglycan (HSGAG) binding site (HBS) within the FGF core is composed of the β1-β2 loop and parts of the region spanning β10 and β12. For paracrine FGFs, the elements of the HBS form a contiguous, positively charged surface. By contrast, the HBS of the FGF19, FGF21 and FGF23 subfamily contains ridges formed by the β1-β2 loop and the β10-β12 region that sterically reduce HSGAG binding to the core backbone of the FGFs and lead to the endocrine nature of this subfamily. In some embodiments, the FGF1 is administered with heparin or is linked to heparin. In some embodiments, the FGF1 is administered with heparan sulfate or is linked to heparan sulfate. Interactions with heparin or heparin sulfate can allow FGF1 diffusion and prevents accumulation due to its interactions with the extracellular matrix. This can be advantageous, for example to promote dispersion in brain tissue after administration to the brain, as opposed to remaining strictly at the site of administration, e.g., bound to the nearby extracellular matrix.

### Anti-Diabetic Activity

As noted above, the minimum, central biological activity and/or biological effect of the FGF1 polypeptides as described herein is lowering abnormally high blood glucose levels when administered to the brain, without causing hypoglycemia, and/or normalizing blood glucose levels. In this context, the term “normalizing blood glucose levels” refers not just to reducing the blood glucose levels to within the normal range, but also maintaining the levels there for a prolonged period of time. Thus, in some embodiments, the FGF1 biological activity is that of an anti-diabetic agent. In some embodiments, the FGF1 polypeptide retains at least 85%, at least 90%, at least 95%, at least 97% or at least 99% of the anti-diabetic activity of human FGF1 of SEQ ID NO:1. The glucose lowering biological activity can be assayed by measuring fasting or fed blood glucose levels by methods known to those skilled in the art. The fasting plasma glucose (FPG) test and the 75-g oral glucose tolerance test (OGTT) are examples of suitable assays for measuring blood glucose levels and/or screening for diabetes. The fasting blood glucose level, which is measured after a fast of at least 8 hours, is the most commonly used indication of overall glucose homeostasis, largely because disturbing events such as food intake are avoided.

As noted above, a normal fasting blood glucose level is between 70 and 100 mg/dL. Blood glucose levels will rise after food is ingested, but will normally be less than 140 mg/dL two hours after eating. A fasting blood glucose level between 100 and 125 mg/dL or any value between 140 and 199 mg/dL during a two hour 75 g oral glucose tolerance test is considered to be a marker of pre-diabetes. An individual is considered diabetic if they have two consecutive fasting blood glucose tests greater than 126 mg/dL, any random blood glucose test level greater than 200 mg/dL, or a two hour 75 g oral glucose tolerance test with any level over 200 mg/dL. Methods to measure the glucose levels in a sample of blood are known in the art. For instance, blood glucose can be measured in a sample of blood taken from a vein or from a small finger stick sample of blood. It can be measured in a laboratory either alone or with other blood tests, or it can be measured using a handheld glucometer, which permits frequent monitoring of blood glucose levels without the need for a doctor's office or laboratory.

The anti-diabetic biological activity of FGF1 administration to the brain is demonstrated herein in rodent models of type 2 diabetes (T2D). FGF1 and FGF21 can also transiently reduce blood glucose levels in diabetes when administered systemically (11,12). The role of FGF21 in metabolic regulation was discovered in association with its adipocyte-specific ability to cause glucose uptake, which is accomplished in part by upregulating transcription of the glucose transporter GLUT1 (3). FGF21 stimulates glucose uptake into adipocytes in an insulin-independent fashion. Systemic administration of FGF1 elicits a glucose lowering effect that is transient, albeit longer in duration (up to 42 h) (21) than that elicited by either FGF19 (7) or FGF21(22). Methods and compositions suitable to achieve this anti-diabetic effect upon systemic administration of FGF1, and fragments and mutant forms that achieve the systemic effect are described in reference (37, 38, 39). However, the FGF administration in these studies failed to maintain a biological effect of sustained blood glucose normalization or diabetic remission. As demonstrated in the Examples herein, normalization of fed and fasting blood glucose levels can be established for 18 weeks or more, and possibly indefinitely, with a single unit dose of FGF1 administered to the brain. There is no reason to believe that normalization maintained for 18 weeks, the longest period examined in the studies detailed herein, would suddenly end at week 19 (i.e., 4¾ months), or for that matter, longer. The term “prolonged period” as used herein refers to at least 1 week of blood glucose normalization on a single unit dose administration of FGF1 polypeptide, and can be, for example, at least 2 weeks, at least 3 weeks, at least 4 weeks (or one month), at least 5 weeks, at least 6 weeks, at least 7 weeks, at least 8 weeks (or two months), at least 9 weeks, at least 10 weeks, at least 11 weeks, at least 12 weeks (or 3 months), at least 13 weeks, at least 14 weeks, at least 15 weeks, at least 16 weeks (or 4 months), at least 17 weeks, at least 18 weeks, at least 20 weeks (5 months), at least 6 months, at least 1 year or more. In some embodiments the methods described herein induce sustained diabetic remission (defined as maintaining the blood glucose levels within the normal range for a prolonged period) after a single unit dose administration of FGF1 polypeptide. To the extent necessary if regular glucose monitoring shows the blood glucose normalizing effect diminishing over time, repeated administration of FGF1 polypeptide to the brain can be performed to re-establish or maintain the blood glucose regulation or normalization effect.

It is contemplated that preparations of the FGF1 molecules and fragments described in reference (37,38,39), which are incorporated herein by reference in their entireties, can be used in methods described herein to achieve a sustained or prolonged blood glucose normalizing or anti-diabetic effect.

In some embodiments the methods of treatment described herein prevent toxicities or side effects, e.g., hypoglycemia and/or loss of body weight and/or reduction of food intake. In some embodiments the administration of FGF1 polypeptide to the brain can increase the rate of peripheral glucose clearance (a measure of the efficiency of glucose removal from the circulation relative to rate in absence of the treatment) in an individual with disease. The treatment can increase the glucose clearance rate by at least 1.5 fold, at least 2-fold or at least 3-fold or more. In some embodiments the increase in rate of glucose clearance occurs in the basal state, i.e. a change in glucose clearance without change in hepatic glucose production, glucose tolerance, insulin secretion or insulin sensitivity. In some embodiments the treatment lowers the blood glucose levels and/or increases rate of glucose clearance without change in circulating levels of glucoregulatory hormones, e.g., insulin (which promotes absorption of glucose from the blood to skeletal muscles and fat tissue), glucagon (which stimulates the liver to convert glycogen to glucose thereby increasing hepatic glucose production), corticosterone (antagonist of insulin). In some embodiments, the methods of treatment increase the hepatic glycogen content, levels of glucoregulatory enzymes e.g., glucokinase (GCK), liver-type pyruvate kinase (L-PK), glycogen synthase relative to absence of treatment in an individual with disease. In some embodiments the treatment of high blood glucose levels, diabetes, metabolic disorders occur by increasing hepatic glucose uptake, glycogen synthesis and glycolysis relative to the levels in an untreated individual having high blood glucose levels. In some embodiments, the methods of treatment are not associated with reduction in plasma levels of triglycerides (TG), cholesterol (Chol), or non-esterifed fatty acids (NEFA).

### Administration to the Brain

Disclosed herein are compositions and methods for lowering elevated blood glucose levels in a subject by administration of a unit dosage of FGF1 polypeptide to the brain. Effective biological activity of FGF1 in the brain by its localized and controlled administration to the tissue, results in blood glucose normalization and sustained diabetes remission. As noted above and demonstrated in the Examples provided herein, the sustained diabetes remission is obtained at an FGF1 dosage that is much lower than that required for its systemic efficacy and reduces the risk of side effects posed by other conventional therapies for metabolic disorders.

Effective administration and uptake of a therapeutic agent to the brain is hindered by two barriers to brain delivery; (a) the blood brain barrier—is a unique membranous barrier that segregates the brain from the circulating blood and (b) blood-cerebrospinal fluid barrier—a barrier located at the tight junctions that surround and connect the cuboidal epithelial cells on the surface of the choroid plexus. Systemic administration of FGF1 can result in lowering of blood glucose levels for up to 42 hrs (37,38,39,21). This is longer than can be achieved, for example, with insulin, but not nearly as long as is demonstrated herein via administration to the brain. While systemic administration of FGF1 can result in its delivery to the brain, the transferred dosage can depend, among other factors, upon its rate of transfer from the blood to the brain, or distribution between blood and brain, effective interactions between FGF1 and its receptors and amount of FGF1 available for uptake in relation to its systemic clearance. As noted above, FGFs tend to be “sticky,” binding to heparan sulfate in the extracellular matrix, which sharply limits the circulation of FGF polypeptides administered systemically, e.g., intravenously or subcutaneously. These factors lead to delivery of an ineffective dose to the brain or conversely require a larger systemic dose to achieve required therapeutic levels in the brain, thereby increasing the risk of toxicities or other unwanted effects. Methods of drug administration are known in the art for effective transfer of drugs to the brain (40). Non limiting examples are detailed herein.

In some embodiments the FGF1-polypeptide is directed to the brain by intracerebroventricular administration. Delivery of drugs by lumbar puncture or direct intraventricular injection can bypass the blood-brain barrier by direct introduction into the CSF. The layers of cells that line the fluid spaces of the brain are permeable to molecules introduced this way. Controlled-release formulations and drug-delivery devices can be used after a single dose is given by lumbar puncture or by cerebrointraventricular injection. For example, Depot cytarabine (DTC 101) contains the drug cytarabine encapsulated in microscopic spherical particles and has extended use of the therapeutic drug concentrations after a single dose given by lumbar puncture or by intraventricular injection. While extended release formulation was not needed to achieve at least 18 weeks of blood glucose normalization in studies described herein, administration in such extended release formulations is contemplated to further extend the effect, if necessary or desired. Extended release formulations are contemplated as potentially useful for any mode of administration to the brain described herein.

In some embodiments, the drug is administered directly to the brain interstitium by intracranial administration.

In some embodiments, the pharmaceutical composition is contained in an implantable pump. In this mode, an intraventricular catheter is surgically implanted to deliver a drug directly into the brain, and accordingly in one aspect, the pharmaceutical composition containing FGF 1 polypeptide is contained in a catheter. Various catheters are inserted into the brain (lumbar subarachnoid space, cisterns, and ventricles). These can be connected to reservoirs and pumps and can be left in place during the duration of therapy for continuous or pulsatile drug infusions. In some embodiments, the pharmaceutical composition is contained in an implantable pump and permits drug delivery, for example, directly to the brain interstitium. The pump can be designed to deliver to the intended site of action, at the required rate of administration, and in the proper therapeutic dose. One example of such pump includes but is not limited to the commercially available, the Alzet osmotic mini pump to delivering drugs at a controlled rate and dose over extended periods within the central nervous system. A variety of pumps have been designed to deliver drugs from an externally worn reservoir through a small tube into the central nervous system. The Ommaya reservoir is another example, in which an intraventricular catheter is connected to a drug reservoir implanted under the scalp. This technique, however, does not achieve truly continuous drug delivery. More recently, several implantable pumps have been developed that possess several advantages over the Ommaya reservoir. They can be implanted subcutaneously and connected to an intraventricular catheter and refilled by subcutaneous injection and are capable of delivering drugs as a constant infusion over an extended period of time. Furthermore, the rate of drug delivery can be varied using external handheld computer control units.

In some embodiments, the FGF1-polypeptide can be contained in a continuous flow pump. The delivery mechanism of one such pump is based on the expansion of Freon gas at 37° C. that pushes a diaphragm “plunger/pusher” plate. Usually, the pump reservoir is implanted subcutaneously and is connected to a catheter implanted into the nervous system to deliver the therapeutic molecules. The reservoirs are refilled by subcutaneous injection of the solution containing the FGF1 polypeptide composition. An example of the use of such a pump in clinical practice is the intrathecal pump delivery of the GABAergic drug baclofen for spasticity.

In some embodiments, the FGF1 polypeptide can be contained in a programmable pump. Programmable pumps include electromechanical pumps of the peristaltic type, powered by batteries. Their built-in electronics can be remotely controlled from an external programming unit. An example is the SynchroMed system (Medtronic Inc.). The infusion can be programmed in various modes: continuous hourly infusions, repeated bolus infusions with a specified delay, multiple doses over a programmed interval, or a single bolus infusion. Non-limiting examples of pumps available for interstitial central nervous system drug delivery include, the Infusaid™ pump, which also uses the vapor pressure of compressed Freon to deliver a drug solution at a constant rate; the MiniMed™ PIMS system which uses a solenoid pumping mechanism, and the Medtronic SynchroMed™ system delivers drugs via a peristaltic mechanism. The distribution of small and large drug molecules in the brain can be enhanced by maintaining a pressure gradient during interstitial drug infusion to generate bulk fluid convection through the brain interstitium or by increasing the diffusion gradient by maximizing the concentration of the infused agent as a supplement to simple diffusion. Another recent study shows that the epidural (EPI) delivery of morphine encapsulated in multivesicular liposomes (DepoFoam drug delivery system) produced a sustained clearance of morphine and a prolonged analgesia, and the results suggest that this delivery system is without significant pathological effects at the dose of 10 mg/ml morphine after repeated epidural delivery in dogs. Such systems can be adapted to deliver FGF1 polypeptides as described herein to the brain.

In some embodiments, the pharmaceutical composition comprising FGF1 polypeptide is contained in a syringe, including a blunt tip syringe for injection to the brain or to the nasal passages. Microspheres can be implanted stereotactically in the brain. Stereotactic procedures on the brain involve guiding a probe into discrete and precise target areas based on anatomical and functional landmarks without causing damage to the surrounding structures. Currently, this method is most frequently applied for the treatment of brain tumors and neurodegenerative disorders such as Parkinson disease, but it can be adapted to deliver FGF1 polypeptide preparations to the brain.

Delivery to and uptake of therapeutics to the brain is favored by low molecular weight, lack of ionization at physiological pH and lipophilicity (40). Therefore one possible strategy to improve brain targeting is to modify the drug to increase its lipophilicity.

### Liposomes

Liposomes are vesicular structures with an aqueous core surrounded by a hydrophobic lipid membrane created by extrusion of phospholipids and known in the art to be used for drug delivery purposes.

In some embodiments of the compositions and methods described herein, the FGF1 is encapsulated in a liposome. Liposomes can vary in size from 15 nm to 100 μm and are contemplated to have either a single layer (uni-lamellar), or multiple phospholipid bilayer membranes (mutilamellar structure). In one aspect, the FGF1 polypeptide can be encapsulated in a niosome, a non-phospholipid-based synthetic vesicle. Liposome compositions can be prepared by a variety of methods that are known in the art. See e.g., reference (25,26,27,28, 29).

Micelles

In one aspect, the FGF1 polypeptide is encapsulated in a micelle. Micelles are spherical aggregates of amphiphilic molecules dispersing in water with their hydrophilic head groups on the surface of the sphere, and their hydrophobic tails collected inside. An important property of micelles is their ability to increase the solubility and bioavailability of poorly soluble pharmaceuticals. The amphiphilic molecules in micelles are in constant exchange with those in the bulk solution. On the other hand, polymeric micelles, also known as polymersomes, are self-assembled polymer shells composed of block copolymer amphiphiles such as polyethylene glycol-polylactic acid (PEG-PLA) and PEG-polycaprolactone (PEG-PCL). Polymeric micelles differ from nanoparticles that are either more solid or monolithic (nanospheres) or contain an oily or aqueous core and are surrounded by a polymer shell (nanocapsules). However, in practice, polymeric micelles also be referred to as nanoparticle or nanocarriers because of their particle size. Accordingly, in some embodiments the FGF1 polypeptide is encapsulated in a nanoparticle. In some embodiment, the FGF1 polypeptide can be encapsulated in a microcapsule or a microsphere, which are free flowing powders consisting of spherical particles of 2 millimeters or less in diameter, usually 500 microns or less in diameter. Reference (41) teaches the formation and use of such microspheres encapsulating a drug as an injectable drug delivery system to target drugs to the brain.

### Nanoparticles

Nanoparticles are solid matrix colloidal particles with diameters ranging from 1-1000 nm formed using various polymers like degradable starch, dextran, chitosan, microcrystalline cellulose (MCC), hydroxypropyl cellulose (HPC), hydroxypropyl ethylcellulose (HPMC), carbomer, and wax-like starch, gelatin polymers. In these carrier systems, the drug can be loaded via either incorporation with the system or its adsorption on the particulate system. The encapsulating nanoparticle can be, for example, solid-lipid nanoparticles (SLNs), polymeric nanoparticles, or oil-in-water nanoemulsions. Solid-lipid nanoparticles are surfactant-stabilized aqueous colloidal dispersions of lipid nanoparticles that solidify upon cooling. They contain a lipid phase dispersed in an aqueous environment (42). Polymeric nanoparticles are solid colloidal particles created from polymeric systems. These nanoparticles are made from biocompatible polymers that encapsulate or adsorb drugs for prolonged release (42). Nanoemulsions are oil-in-water (O/W) or water-in-oil (W/O) formulations made with edible or otherwise pharmaceutically acceptable oils, surface-active agents (surfactants), and water, where the diameter of the inner phase is reduced to nanometer length scale. The versatility of nanoemulsions is based on the different types of oils and surface modifiers that can be used. For instance, oils that are rich in omega-3 polyunsaturated fatty acids (PUFA) can play a very important role in overcoming biological barriers, including the BBB (see, e.g., reference 43).

For effective targeting, the liposomes and nanoparticles encapsulating the FGF1 polypeptide can be further linked with and/or coated with other agents. One example of such an agent as described in reference 44, can be an antibody binding fragment such as Fab, F(ab′)2, Fab′ or a single antibody chain polypeptide which binds to a receptor molecule present on the vascular endothelial cells of the mammalian blood-brain barrier. The receptor is preferably of the brain peptide transport system, such as the transferrin receptor, insulin receptor, IGF-I or IGF-2 receptor. The antibody binding fragment is preferably coupled by a covalent bond to the liposome. Another example is direct or indirect covalent linkage of apolipoprotein e to nanoparticles encapsulating FGF1. Such linkage can lead to effective brain delivery of the linked agent, as described in reference 34 (covalent linkage of apoliporotein e to albumin). In some embodiments, the nanoparticles encapsulating the FGF1 polypeptide can be coated with poly(ethylene glycol) or polysorbate 80 or albumin or its functional groups. PEG-containing surfactants, poly(oxy-ethylene)-poly(oxy-propylene) can also be used for coating nanoparticles. Poly(ethylene glycol)-modified SLNs have been shown to penetrate the BBB and allow for greater delivery of drug to the CNS (45). Polysorbate 80-coated poly(n-butylcyanoacrilate) nanoparticles have been formulated by emulsion polymerization method to target selectively rivastigmine or tacrine to the brain for Alzheimers disease. Coating of nanoparticles with 1% polysorbate 80 increased the concentrations of the drug in the brain when compared with the free drug, indicating potential selective targeting to the CNS (46). Nanoparticles and liposomes can also be linked to carrier peptides for examples TAT, to improve their lipophilicity. Liposomes prepared using cholesterol-PEG2000-TAT enhanced delivery of liposomes to the brain.

### Carrier Peptides

In some embodiments, the FGF1 polypeptide is modified by linkage to a carrier peptide which by itself is capable of crossing the blood brain barrier by transcytosis. In reference 47, Pardridge describes the preparation of chimeric peptides by coupling or conjugating the pharmaceutical agent to a transportable peptide. The chimeric peptide purportedly passes across the barrier via receptors for the transportable peptide. Accordingly, in some embodiments the FGF1 polypeptide is fused to a carrier peptide. Non-limiting examples of such transportable peptides, or vectors, suitable for coupling to the pharmaceutical agent include transferrin, insulin-like growth factors I and II, basic albumin and prolactin. The conjugation can be carried out using bifunctional reagents which are capable of reacting with each of the polypeptides and forming a bridge between the two. The preferred method of conjugation involves polypeptide thiolation, wherein the two polypeptides are treated with a reagent such as N-succinimidyl 3-(2-pyridyldithio)propionate (SPDP) to form a disulfide bridge between the two polypeptides. Other known conjugation agents can be used, so long as they provide linkage of the two polypeptides (i.e. therapeutic polypeptide drug and the transportable peptide) together without denaturing them. Preferably, the linkage can be easily broken once the chimeric polypeptide has entered the brain. Reference (35) teaches the use of an inert fragment of insulin as a peptide carrier to transport a therapeutic polypeptide across the blood brain barrier. Reference (48) teaches targeting biotinylated FGF2 to the brain by linking it to a carrier peptide for example insulin, transferrin, insulin-like growth factor, leptin, low density lipoprotein (LDL), and monoclonal antibodies that bind to insulin, IGF, leptin or LDL receptor on the blood brain barrier, and with avidin and streptavidin. In some embodiments, the FGF1-polypeptide can be linked to short cell penetrating peptides, which have the ability to cross cell membrane bilayers. Non limiting examples of such peptides include TAT (HIV-1 transactivating transcriptor) SynB3, Tat 47-57, transportan as in reference (49).

In some embodiments of the technology described herein, the FGF1 therapeutic peptide can be chemically modified by linking to a lipophilic molecular group to increase its lipophilicity. Examples of such modifications include, among others, esterification, or amidation of the hydroxy-, amino-, or carboxylic acid-groups of the polypeptide. Lipophilic molecular groups can comprise lipid moieties such as fatty acid, glyceride or phospholipids.

In some embodiments, the drug is targeted to the brain via intranasal administration. Drugs administered intransally are transported along the olfactory sensory neurons to yield significant concentrations in the cerebrospinal fluid and olfactory bulb and hence intransal administration can be an alternative, non-invasive route for targeting the therapeutic polypeptides to the brain. As proof of principle, three peptides, melanocortin, vasopressin and insulin, were administered intranasally and found to achieve direct access to the cerebrospinal fluid (CSF) within 30 minutes, bypassing the bloodstream (66). Nasal administration of two L-dopa butyl ester drugs resulted in higher CSF levels of L-dopa than those observed after intravenous administration (67). In these examples, the percentage of the applied dose that passes to the brain and CSF is about 2% to 3%. Thus, unit dosages of FGF1 polypeptides for intranasal administration can be adjusted upwards to take this into account. Alternatively, or in addition, efficiencies can be increased by combination or conjugation with agents or excipients that promote absorption to the olfactory neurons. Preferential uptake of intranasally administered apomorphine directly into the cerebral spinal fluid has been demonstrated in a phase I study, and this opens the possibility of treating neurologic disorders with intranasal apomorphine. These examples indicate that a nasal route can be a viable method for the delivery of peptides, analgesics, and other drugs.

In the intranasal mode of administration, when absorption in the brain is the goal, the olfactory nerve is the target as it is the site where the central nervous system is directly expressed on the nasal mucosal surface. Therefore, in order to enhance the absorption of a therapeutic drug or agent into the olfactory neurons, the drug or agent should be capable of at least partially dissolving in the fluids that are secreted by the mucous membrane that surround the cilia of the olfactory receptor cells of the olfactory epithelium. Additionally, it is preferred that the drug or agent exhibits minimal effects systemically and is administered in a unit dose which results in effective levels of its activity in the brain without undue toxicities. Therefore the therapeutic peptide can be linked to a carrier that increases its dissolution within nasal secretions. Non-limiting examples of such carriers include GM-1 ganglioside, phosphotidylserine (PS), and emulsifiers such as polysorbate 80. Linkage with lipophilic carriers such as gangliosides or phosphotidylserine can improve the adsorption of the therapeutic drug into the olfactory neurons and through the olfactory epithelium. Frey et al. teaches a method of transporting insulin and fibroblast growth factors to the brain using a composition comprising ganglioside and/or phosphotidylserine (reference 50 and 51). In some embodiments, the FGF1-polypeptide formulated for intranasal administration also comprises a ganglioside or a phosphatidylserine.

The therapeutic drug to be targeted to the brain formulated for intranasal administration can also be linked to another neural peptide or its fragment which can assist in transporting the therapeutic agent to the brain. Non limiting examples of such neural peptides include brain-derived neurotropic factor, insulin, and insulin like growth factors. In one embodiment, the FGF1 polypeptide can be combined with or formulated within micelles comprised of lipophilic carriers. In some embodiments, the FGF1 polypeptide for intranasal administration can be encapsulated in nanoparticles, liposomes, micelles, microspheres, niosomes, cyclodextrin-inclusion complexes, or nanoemulsions.

Chitosan (CS) is a β-(1-4)-linked D-glucosamine and N-acetyl-D-glucosamine co-molecule, which represents a linear backbone structure linked through glycosidic bonds. Chitosan nanoparticles showed a significant increase in the drug concentration in the CSF after intranasal administration in rats. The nanoparticles can be coated with polymers such as polyethylene glycol-polylactic acid (PEG-PLA). Methods for preparation of (PEG-PLA) nanoparticles are described, for example, in reference (50). The chitosan nanoparticles can be complexed with cyclodextrins. See, e.g., reference 51 and 52, which describe a pharmaceutical composition comprising cyclodextrins and/or a disaccharide and/or sugar alcohol for intranasal administration of apomorphine and Galanin-like peptide (GALP), respectively. The term “cyclodextrins” refers to cyclic oligosaccharides, like α-, β- and γ-cyclodextrin and their derivatives, preferably β-cyclodextrin and its derivatives, preferably methylated β-cyclodextrin, with a degree of CH3-substitution between 0.5 and 3.0, more preferably between 1.7 and 2.1. The term “saccharides” refers to disaccharides, like lactose, maltose, saccharose and also refers to polysaccharides, like dextrans, with an average molecular weight between 10,000 and 100,000, preferably 40,000 and 70,000. The term “sugar alcohols” refers to mannitol and sorbitol. In some embodiments, the FGF1 polypeptide formulation formulated for administration via an intransal route further comprises saccharides selected from the group consisting of cyclodextrins, disaccharides, polysaccharides and combinations thereof. Drugs can be encapsulated in carriers, like cyclodextrins inclusion complexes containing a hydrophobic core and a hydrophilic shell which can help improve upon the drug solubility problems and improve brain uptake after intranasal administration. Specific targeting to olfactory epithelium for these drugs can be achieved by using ulex europeus aggutinin 1 (UEA 1), which has specific binding affinity to 1-fructose residues found on the apical surface of the olfactory epithelium. In some embodiments, the FGF1 polypeptide composition also comprises, UEA 1.

The compositions can be dispensed intranasally as a powdered or liquid nasal spray, nose drops, a gel or ointment, injection or infusion contained in a tube or catheter, by syringe, by pledge, or by submucosal infusion. Also the composition can made viscous using vehicles such as natural gums, methylcellulose and derivatives, acrylic polymers (carbopol) and vinyl polymers (polyvinylpyrrolidone). Many other excipients, known in the pharmaceutical literature, can be added, such as preservatives, surfactants, co-solvents, adhesives, anti-oxidants, buffers, viscosity enhancing agents, and agents to adjust the pH or the osmolarity.

Nasal powder compositions can be made by mixing the active agent and the excipient, both possessing the desired particle size. Other methods to make a suitable powder formulation can be selected. Firstly, a solution of the active agent and the cyclodextrin and/or the other saccharide and/or sugar alcohol is made, followed by precipitation, filtration and pulverization. It is also possible to remove the solvent by freeze drying, followed by pulverization of the powder in the desired particle size by using conventional techniques, known from the pharmaceutical literature. The final step is size classification for instance by sieving, to get particles that are less than 100 microns in diameter, preferably between 50 and 100 microns in diameter. Powders can be administered using a nasal insufflator. Powders may also be administered in such a manner that they are placed in a capsule. The capsule is set in an inhalation or insufflation device. A needle is penetrated through the capsule to make pores at the top and the bottom of the capsule and air is sent to blow out the powder particles. Powder formulation can also be administered in a jet-spray of an inert gas or suspended in liquid organic fluids. In some embodiments, the FGF1 polypeptide composition for intranasal administration can be adapted for aerosolization and inhalation. The composition can be administered nasally via pressurized aerosol, aqueous pump spray or other standard methods known to those skilled in the art.

The composition of FGF1 polypeptide can be administered in the form of spray in a non-pressurized aerosol device, for example a Pfeiffer pump. To deliver the therapeutic agent to the olfactory neurons, the composition formulated from intranasal administration can be administered to the olfactory area located in the upper third of the nasal cavity. In some embodiments, the compositions for nasal administration can be contained in a syringe, catheter, an inhaler, a nebulizer, a nasal spray pump, a nasal irrigation pump, or a nasal lavage pump. Non-limiting example for intranasal administration of liquid formulation can include (a) delivering drops with a drop pipette, (b) rhinyle catheter and squirt tube which involves inserting the tip of a fine catheter or micropipette to the desired area under visual control and squirt the liquid into the desired location, (c) squeeze bottles which involve squeezing a partly air-filled plastic bottle, to deliver the atomized drug from a jet outlet, (d) metered-dose spray pumps which deliver a unit dose of drug per use, (e) single- and duo-dose spray devices which is used for a single administration of a unit dose of drug, (f) nasal pressurized metered-dose inhalers delivers a nasal aerosol preparation, and (g) powered nebulizers and atomizers to administer drug in the form of a mist. Non-limiting examples for intranasal administration of liquid formulations can include nasal powder inhalers (e.g., Rhinocort Turbuhaler®; BiDose™/Prohaler™) from Pfeiffer/Aptar), nasal powder sprayers (e.g., Fit-lizer™ device, Unidose-DP™), and nasal powder insufflators (e.g., Bi-Directional™ nasal delivery, Optinose).

While intranasal administration is appealing for its relative non-invasiveness, in some embodiments, the administration is not via the intranasal route—that is, in some embodiments, the intranasal route is excluded and another route of administration to the brain is employed.

### Metabolic Disorders

In some embodiments, the compositions and methods described herein can be used to treat metabolic disorders, e.g., type 2 diabetes, gestational diabetes, drug-induced diabetes, high blood glucose, metabolic syndrome, insulin resistance, type 1 diabetes and conditions and symptoms related thereto. In some embodiments the metabolic disorder is characterized by or involves abnormally elevated blood glucose levels. In some embodiments the FGF1 polypeptide can be used to treat conditions related to metabolic disorders including e.g., hypertension (high blood pressure), hyperglycemia, cardiovascular disease, obesity, hypertriglyceridemia and/or reduced high-density lipoprotein cholesterol (HDL-C).

The metabolic syndrome, variously referred to as ‘Syndrome X,’ the ‘Deadly Quartet’ and the ‘Insulin Resistance Syndrome’ is a cluster of the most dangerous cardiovascular disease risk factors: diabetes and prediabetes, abdominal obesity, high cholesterol and high blood pressure. The NHLBI, AHA, International Diabetes Foundation (IDF), and others have proposed a harmonized guideline for diagnosis of metabolic syndrome. Under these guidelines, metabolic syndrome is diagnosed when a patient has at least 3 of the following 5 conditions: (1) Fasting blood glucose ≧100 mg/dL (or receiving drug therapy for and/or diagnosed with hyperglycemia or type 2 diabetes); (2) Blood pressure ≧130/85 mm Hg (or receiving drug therapy for and/or diagnosed with hypertension); (3) Triglycerides ≧150 mg/dL (or receiving drug therapy for and/or diagnosed with hypertriglyceridemia); (4) HDL-C<40 mg/dL in men or <50 mg/dL in women (or receiving drug therapy for reduced HDL-C), (5) Waist circumference ≧102 cm (40 in) in men or ≧88 cm (35 in) in women; if Asian American, ≧90 cm (35 in) in men or ≧80 cm (32 in) in women (receiving therapy for and/or diagnosed with obesity). Elevated total cholesterol levels may be related to metabolic syndrome. Elevated LDL cholesterol is marked by levels above about 100, about 130, about 160, or about 200 mg/dL. It is contemplated that administration of FGF1 polypeptide to the brain as described herein can be beneficial to subjects with metabolic syndrome, and that FGF1 administration as described herein can be beneficial in combination with one or more drugs or treatments administered for the treatment of metabolic syndrome or its symptoms.

Metabolic syndrome can be associated with microalbuminuria (urinary albumin excretion ratio ≧20 μg/min or albumin:creatinine ratio ≧30 mg/g). It can also be associated with hyperuricemia (uric acid in the blood above the normal range of 360 μmol/L (6 mg/dL) for women and 400 μmol/L (6.8 mg/dL) for men). It can also be associated with fatty liver disease and conditions related thereto e. g. alcoholic steatosis or nonalcoholic fatty liver disease (NAFLD), alcoholic steatohepatitis (part of alcoholic liver disease) and non-alcoholic steatohepatitis (NASH). Fatty liver disease, and therefore metabolic syndrome, can also be associated with abetalipoproteinemia, glycogen storage diseases, Weber-Christian disease, acute fatty liver of pregnancy and lipodystrophy. Metabolic syndrome can also be associated with polycystic ovarian syndrome (in women), and acanthosis nigricans.

Metabolic syndrome can be associated with a pro-inflammatory state diagnosed by elevated high sensitivity C-reactive protein, e.g., above 10 mg/L, elevated inflammatory cytokines (e.g., TNFα, IL-6), and a decrease in adiponectin plasma levels. Metabolic syndrome can be associated with a pro-thrombotic state, diagnosed by measurement of fibrinolytic factors (PAI-1, etc.) and clotting factors (fibrinogen, etc.).

The most commonly used drugs for elevated triglycerides and reduced HDL-C are fibrates and nicotinic acid. High-dose ω-3 fatty acids can also be administered to reduce serum triglycerides.

In some embodiments the methods and compositions described herein can be used to treat obesity, reduce percentage body fat or total body fat. Obesity contributes to hypertension, high serum cholesterol, low HDL-c and hyperglycemia, and is associated with higher cardiovascular disease risk. Obesity can be diagnosed by an increase in body mass index (BMI), but an excess of body fat in the abdomen, measured simply by waist circumference, and measurement of waist-hip ratio is more indicative of the metabolic syndrome profile than BMI. In some embodiments, the methods can be used to treat elevated waist-hip ratio, elevated body mass index, elevated body fat percentage, elevated waist circumference, elevated fat to muscle ratio class I obesity, class II obesity, and/or class III obesity. Class I obesity is characterized by a BMI of about 30 to 35, class II obesity is characterized by a BMI of about 35 to about 40 and class III obesity, also referred to as morbid obesity, is characterized by a BMI of 40 or greater. A BMI of about 45 or above is considered super obese. Elevated waist-hip ratio is defined as greater than about 0.7 for women, and greater than 0.9 for men.

Insulin resistance appears to be a primary mediator of metabolic syndrome and the major characteristic of type 2 diabetes. Insulin promotes glucose uptake in muscle, fat, and liver cells and can influence lipolysis and the production of glucose by hepatocytes. Insulin resistance is defined as a condition in which cells in the body (and especially liver, skeletal muscle and adipose/fat tissue) become less sensitive and/or fail to respond to normal actions of insulin and eventually become resistant to insulin. Under these conditions, glucose can no longer be absorbed by the cells but remains in the blood, triggering the need for more insulin (hyperinsulinemia). Once the pancreas is no longer able to produce enough insulin, a subject becomes hyperglycemic and can be diagnosed with type 2 diabetes. Contributors to insulin resistance include abnormalities in insulin secretion and insulin receptor signaling, impaired glucose disposal, and elevated proinflammatory cytokines. These abnormalities, in turn, may result from obesity with related increases in free fatty acid levels and changes in insulin distribution (insulin accumulates in fat). Insulin resistance therefore is strongly associated with dysregulation of glucose and lipid metabolism (dyslipidemia). In some embodiments, the methods and compositions described herein can be used to treat the symptoms and conditions related to insulin resistance.

Methods to measure insulin resistance are known in art. One example includes the hyperinsulinemic euglycemic clamp, which to measures the amount of glucose necessary to compensate for an increased insulin level without causing hypoglycemia. After an overnight fast, insulin is infused intravenously at a constant rate that may range from 5 to 120 mU·m-2·min-1 (dose per body surface area per minute). This constant insulin infusion results in a new steady-state insulin level that is above the fasting level (hyperinsulinemic). As a consequence, glucose disposal in skeletal muscle and adipose tissue is increased, whereas hepatic glucose production (HGP) is suppressed. Under these conditions, a bedside glucose analyzer is used to frequently monitor blood glucose levels at 5- to 10-min intervals while 20% dextrose is given intravenously at a variable rate to “clamp” blood glucose concentrations in the normal range (euglycemic). An infusion of potassium phosphate is also given to prevent hypokalemia resulting from hyperinsulinemia and increased glucose disposal. After several hours of constant insulin infusion, steady-state conditions can typically be achieved for plasma insulin, blood glucose, and the glucose infusion rate (GIR). Assuming that the hyperinsulinemic state is sufficient to completely suppress HGP, and since there is no net change in blood glucose concentrations under steady-state clamp conditions, the GIR must be equal to the glucose disposal rate. Thus, whole body glucose disposal at a given level of hyperinsulinemia can be determined directly. Glucose may be labeled with commonly-used tracers, 3-3H glucose (radioactive), 6,6 2H-glucose (stable) and 1-13C Glucose (stable).

Insulin resistance can also be measured via the insulin suppression test (IST). After an overnight fast, somatostatin (250 μg/h) or the somatostatin analog octreotide (25 μg bolus, followed by 0.5 μg/min) (74) is intravenously infused to suppress endogenous secretion of insulin and glucagon. Simultaneously, insulin (25 mU·m-2·min-1) and glucose (240 mg·m-2·min-1) are infused into the same antecubital vein for 3 h. From the contralateral arm, blood samples for glucose and insulin determinations are taken every 30 min for 2.5 h and then at 10-min intervals from 150 to 180 min of the IST. The constant infusions of insulin and glucose will determine steady-state plasma insulin (SSPI) and glucose (SSPG) concentrations. The steady-state period is assumed to be from 150 to 180 min after initiation of the IST. SSPI concentrations are generally similar among subjects. Therefore, the SSPG concentration will be higher in insulin-resistant subjects and lower in insulin-sensitive subjects; i.e., SSPG values are inversely related to insulin sensitivity. The IST provides a direct measure (SSPG) of the ability of exogenous insulin to mediate disposal of an intravenous glucose load under steady-state conditions where endogenous insulin secretion is suppressed. Additional non limiting examples include using the quantitative insulin sensitivity check index or the homeostatic model assessment, the details of which are known to those skilled in the art.

A statistically significant change and/or an improvement by at least 10% or more in a clinical measure of insulin resistance is considered effective treatment for insulin resistance.

Methods of diagnosing elevated blood glucose levels and/or glucose intolerance and/or diabetes include, among others, measurement of fasting blood glucose levels and the Oral Glucose Tolerance Test (OGTT). Treatments are considered effective if blood glucose levels are lowered to within the normal range, and preferably maintained within the normal range for at least one week. In the OGTT, after overnight fast, blood samples for determinations of glucose and insulin concentrations are taken at 0, 30, 60, and 120 min following a standard oral glucose load (75 g). The blood glucose levels in this or any other test can be checked with a hand-held glucometer, or measured in a medical laboratory. Glucose assays in clinical use include, but are not limited to assays that use hexokinase, glucose oxidase, or glucose dehydrogenase enzymes using methods known to those of skill in the art. The oral glucose challenge test (OGCT) is a short version of the OGTT, commonly used to screen pregnant women for signs of gestational diabetes. It can be done at any time of day and does not require fasting. The test involves the ingestion of 50 g of glucose, with a blood glucose reading after one hour. A normal response results in a blood glucose level less than or equal to 140 mg/dL at the one hour time point.

The A1c test also known as hemoglobin A1c, HbA1c, or glycohemoglobin test can also be performed for diagnosing elevated blood glucose levels and/or diabetes. The A1C test is based on the attachment of glucose to hemoglobin, and reflects the average of a person's blood glucose levels over the past 3 months. The A1C test result is reported as a percentage. The higher the percentage, the higher a person's blood glucose levels have been. A normal A1C level is below 5.7 percent. An individual with A1C levels within the range of 5.7-6.4% is diagnosed as prediabetic or at a significant risk of developing diabetes. An individual with A1C levels of 6.5 percent or above is diagnosed as diabetic.

Screening for diagnosis of diabetes or risk of diabetes or blood glucose levels can also be done in asymptomatic children using the methods described above. Detection and diagnosis of gestational diabetes (GDM) defined as high blood glucose in women during pregnancy can be carried out by a random blood glucose test, a screening glucose challenge test around 24-28 weeks' gestation, followed by an OGTT if the tests are outside normal range. A diagnosis of GDM can be made if the blood glucose values are greater than or equal to 92 mg/dL, greater than or equal to 180 mg/dL at 1 hr after 75 g OGTT test or greater than or equal to 153 mg/dL at 2 hrs after 75 g OGTT test.

Latent autoimmune diabetes of adults (LADA) describes patients with a type 2 diabetic phenotype combined with islet antibodies and slowly progressive β-cell failure due to body's immune system killing off pancreatic beta cells. Diagnosis can include C-peptide measurement (residual beta cell function by determining the level of insulin secretion (C-peptide), autoantibody panel (which includes detection of glutamic acid decarboxylase autoantibodies (GADA), islet cell autoantibodies (ICA), insulinoma-associated (IA-2) autoantibodies, and zinc transporter autoantibodies (ZnT8). Individuals with LADA typically have low, although sometimes moderate, levels of C-peptide as the disease progresses.

Generally, a subject will be diagnosed with a metabolic disorder prior to treatment for the metabolic disorder. As such, any of the methods of treatment described herein can include the step of first diagnosing a metabolic disorder in the subject.

Metabolic disorders can manifest across several disorders and the methods and compositions described herein are also contemplated to benefit related conditions including, e.g., cardiovascular diseases (e.g., myocardial infarction, angina, pulmonary embolism, high blood pressure, high cholesterol, congestive heart failure), neurological disorders (e.g., stroke, migranes, intracranial hypertension), depression, rheumatologic conditions and orthopedic disorders (e.g., gout, osteoarthritis), dermatological disorders (e.g., acanthosis nigricans), gastrointestinal disorders (e.g., gastroesophageal reflux disease, gallstones), respiratory disorders (e.g., obesity hypoventilation syndrome, asthma), urology and nephrology disorders (e.g., chronic renal failure, erectile dysfunction, urinary incontinence).

### Pharmaceutical Compositions

Described herein are pharmaceutical compositions comprising an FGF1 polypeptide preparation, formulated for administration to the brain. In preferred embodiments, the compositions can be formulated as unit dose preparations for delivering to the brain an amount of an FGF1 polypeptide preparation effective to reduce abnormally high blood glucose levels in an individual to within the normal range. In such embodiments, the unit dose preparation is much less than the dose required to reduce blood glucose levels when FGF1 polypeptide is administered systemically. By “much less” in this context is meant less than 50% of the dose required for systemic blood glucose lowering effect, and includes, for example, less than 40%, less than 30%, less than 25%, less than 20%, less than 15%, and 10% or below. Indeed, as shown in the Examples herein below, a dose of FGF1 that is 10% of that required to provide a transient blood glucose reduction when administered systemically normalizes blood glucose in several different murine models of diabetes for prolonged periods of time when administered to the brain.

Where it is demonstrated in the Examples herein that murine, rat and human FGF1 can provide prolonged blood glucose normalization in a murine model of diabetes when administered to the brain, it is contemplated that the FGF1 polypeptide in a pharmaceutical composition can be a human, rat or mouse FGF1 polypeptide as that term is described herein. Where the function is conserved between human, rat and mouse, it is reasonable to expect that other mammalian species' FGF1 polypeptides will have similar effect, both in those other species and, for that matter, in humans.

FGF1 polypeptide as described herein can be formulated in any of a number of pharmaceutical compositions suitable for administration to the brain. General principles for the preparation of pharmaceutical compositions are described, for example, in the United States Pharmacopeia (U.S.P.), Goodman and Gilman's The Pharmacological Basis of Therapeutics, 10th Ed., McGraw Hill, 2001; Katzung, Ed., Basic and Clinical Pharmacology, McGraw-Hill/Appleton & Lange, 8th ed., Sep. 21, 2000; Physician's Desk Reference (Thomson Publishing; and/or The Merck Manual of Diagnosis and Therapy, 18th ed., 2006, Beers and Berkow, Eds., Merck Publishing Group; or, in the case of animals, The Merck Veterinary Manual, 9th ed., Kahn Ed., Merck Publishing Group, 2005.

The compositions described herein can be administered to the brain by any means known in the art. As the term is used herein, “administered to the brain” refers to modes of administration that deliver administered FGF1 polypeptide to the brain, substantially without reliance on the systemic circulation to deliver the administered polypeptide. Thus, while systemic administration (e.g., intravenous or subcutaneous administration, among others) might be viewed as ultimately delivering a portion of an administered FGF1 polypeptide composition to the brain, such delivery via the systemic circulation is not encompassed by the term “administered to the brain” as it is used herein. It is noted that delivery of an FGF1 polypeptide directly to the brain, e.g, by intracerebroventricular injection, is contemplated to result in some systemic circulation of administered polypeptide that leaves the brain, but where the total amount administered to the brain as described herein will necessarily be much less than the amount necessary for a glucose-lowering effect if administered systemically, the effect of the FGF1 polypeptide administered to the brain will be effectively limited to the effect on the brain. Intranasal delivery, which takes advantage of absorption or uptake by the olfactory neurons, is a form of administration to the brain. As noted, intracerebroventricular (icv) administration is a form of administration to the brain; other forms include, but are not limited to intracranial, intracelial, intracerebellar, and intrathecal administration.

A unit dose of an FGF1 polypeptide composition as described herein can be formulated for delivery via infusion or injection, e.g., local infusion or injection, e.g., via a needle or catheter. While as few as one, single unit dose can be effective for prolonged glucose normalization effect, the FGF1 polypeptide compositions described herein can also be formulated for continuous or prolonged infusion, e.g., via a pump, including, but not limited to an implanted pump, such as an osmotic pump.

Any solvent or diluent acceptable for administration to the brain and otherwise compatible with FGF1 and which do not adversely affect its biological activity can be used to prepare FGF1 polypeptide for administration to the brain. Preparations of FGF1 polypeptide can be dissolved in water, isotonic saline or buffered saline, (e.g., phosphate buffered saline, PBS) and can include a surfactant, e.g., hydroxypropylcellulose, or one from the Tween series of detergents. FGF1 polypeptide can also be prepared in glycerol, liquid polyethylene glycols, and mixtures thereof. All preparations for administration to the brain should be sterile. While generally avoided for administration to the brain, antimicrobial preservatives may be added to the formulation if needed. A unit dose of sterile FGF polypeptide preparation can be dissolved in sterile carrier or solvent prior to administration. An appropriately buffered, isotonic FGF1 polypeptide preparation can be lyophilized to yield a powder for reconstitution in sterile water prior to use. DMSO can be used as solvent to promote or facilitate tissue penetration and thereby increase the amount of FGF1 polypeptide delivered.

In addition to FGFRs, the FGFs also bind to components of the extracellular matrix, heparan sulfate proteoglycans (HPSGs) and their analog, heparin. Formulating the FGF1 with heparin and/or heparin sulfate can minimize this effect such that the FGF1 polypeptide does not bind, for example, to the ventricular ECM, thereby facilitating distribution of the FGF polypeptide in the brain and the therapeutic effect. Furthermore, formulating FGF with heparin and/or heparin sulfate can facilitate the FGF-FGFR interactions upon administration and stabilize FGFs against proteolysis and thermal denaturation.

The FGF family comprises 22 known family members. While FGF1 binds to all known receptors, the binding specificity of individual FGFs and their isoforms is distinct, and different members perform diverse functions. The anti-diabetic effect of FGF19 and FGF21 has been demonstrated previously. Therefore in some embodiments the pharmaceutical composition containing FGF1 also comprises FGF polypeptide or functional fragments of other members of the FGF family, including, but not limited to FGF19 and/or FGF21. The FGF family member(s) administered with FGF1 can either be administered systemically, with FGF1 being administered to the brain, or, for example, administered to the brain, either at the same time and in the same formulation as the FGF1 or in a separately administered dosage form. Delivery to and uptake of therapeutics to the brain is favored by low molecular weight, lack of ionization at physiological pH and lipophilicity. Formulation in lipophilic carriers can also facilitate uptake of FGF1. Therefore the FGF1 polypeptide can be encapsulated in liposomes, micelles, nanoparticles and or modified with a lipophilic molecular group or carrier peptides. Examples and details of these methods are described in previous sections. Lipophilic substances in the form of micelles, liposomes and/or nanoparticles can be added to the pharmaceutical composition to targeting and absorption through the blood brain barrier. The formulation can be contained in a syringe e.g., blunt tip syringe, catheter and or an implantable pump. Sterile injectable solutions can be prepared by incorporating the active compounds, or constructs in the required amount in the appropriate solvent followed by filtered sterilization. Dispersions can be prepared by incorporating the various sterile active ingredients into a sterile vehicle which contains the basic dispersion medium. Sterile powders for reconstitution of sterile injections acan be prepared by vacuum drying, freeze drying, lyophilizing the compositions which will yield a powder of the active ingredient plus any other any additional desired ingredients. More concentrated forms of the compositions described herein are also contemplated.

FGF1-containing pharmaceutical compositions can be delivered via intranasal solutions or sprays, aerosols or powders. Nasal solutions can be aqueous solutions designed to be administered to the nasal passages in drops or sprays. It can be beneficial to prepare formulations for nasal delivery to be similar to nasal secretions. Thus, solutions for nasal delivery can be isotonic and slightly buffered to maintain a pH of 5.5 to 6.5. Such solutions can contain antimicrobial preservatives, similar to those used in ophthalmic preparations. Appropriate stabilizers, if required, can be included in the formulation. A higher viscosity of the formulation increases contact time between the drug and the nasal mucosa thereby increasing the time for permeation. The compositions can made viscous using vehicles such as natural gums, methylcellulose and derivatives, acrylic polymers (carbopol) and vinyl polymers (polyvinylpyrrolidone). Many other excipients, known in the pharmaceutical literature, can be added, such as mucoadhesive excipients include starch, polymers like chitosan, preservatives, surfactants, co-solvents, adhesives, anti-oxidants, buffers, viscosity enhancing agents, and agents to adjust the pH or the osmolarity.

U.S. Pat. No. 6,342,478 incorporated as reference 53 describes various formulations and methods for administering neurologic factors to the brain for the treatment of neurodegenerative diseases, such as Alzheimer's and Parkinson's diseases, or stroke, among other neurological disorders, and is incorporated herein by reference for such teachings. The '478 patent describes the delivery of Nerve Growth Factor (NGF) protein factors to the brain via intranasal application of NGF preparations, and describes direct application of NGF alone to the nasal cavity and application of NGF in combination with other neurologic and or lipophilic agents and/or carriers. Other lipophilic agents include, for example, gangliosides, including GM1 ganglioside, phosphatidylserine. Neurologic agents include, for example, brain-derived neurotrophic factor, insulin and insulin-like growth factors, among others. The same approach can be applied to administer FGF1 to the brain via the intranasal route.

Agents to be administered to the brain through the intranasal route can be delivered to the olfactory epithelium in the olfactory area in the upper third of the nasal cavity. Delivery of FGF1 polypeptide to this area takes advantage of transport of the agents into the peripheral olfactory neurons, rather than into the respiratory epithelium, simultaneously limiting systemic uptake and promoting delivery, via the nasal neurons, of agents to the brain that would not be able to cross the blood-brain barrier from the bloodstream into the brain. Carriers include, for example, lipophilic agents such as ganglioside GM1 and phosphatidyl serine, and emulsifiers such as polysorbate 80. Such agents can enhance the passage of the neurologic factor polypeptides into the olfactory neurons.

The compositions can be dispensed intranasally as a powdered or liquid nasal spray, nose drops, a gel or ointment, injection or infusion contained in a tube or catheter, by syringe, by pledge, or by submucosal infusion. A non-limiting example of a carrier/FGF1 formulation includes, for example, a unit dose of 3 nM FGF1 polypeptide in combination with 30 μM GM-1 ganglioside, and 300 μM phosphatidylserine.

In one embodiment, the FGF1 polypeptide for intranasal administration can be combined with or formulated within micelles comprised of lipophilic carriers. In some embodiments, the FGF1 polypeptide for intranasal administration can be encapsulated in nanoparticles, liposomes, micelles, microspheres, niosomes, cyclodextrin-inclusion complexes, or nanoemulsions. The nanoparticles, liposomes, micelles, microspheres, niosomes, cyclodextrin-inclusion complexes, or nanoemulsions can be functionalized by coating with polymers such a polyethylene glycol and or polysorbate 80. Alternatively the carriers can be formed in the presence of these polymers. The FGF1 polypeptide formulation for administration via an intranasal route can further comprise saccharides selected from the group consisting of cyclodextrins, disaccharides, polysaccharides and combinations thereof. Nasal powder compositions can be made by mixing the active agent and the excipient, both possessing the desired particle size. Other methods to make a suitable powder formulation can be selected. Firstly, a solution of the active agent and the cyclodextrin and/or the other saccharide and/or sugar alcohol is made, followed by precipitation, filtration and pulverization. It is also possible to remove the solvent by freeze drying, followed by pulverization of the powder in the desired particle size by using conventional techniques, known from the pharmaceutical literature. The final step is size classification for instance by sieving, to get particles that are less than 100 microns in diameter, preferably between 50 and 100 microns in diameter. Powders can be administered using a nasal insufflator. Powders may also be administered in such a manner that they are placed in a capsule. The capsule is set in an inhalation or insufflation device. A needle is penetrated through the capsule to make pores at the top and the bottom of the capsule and air is sent to blow out the powder particles. Powder formulation can also be administered in a jet-spray of an inert gas or suspended in liquid organic fluids. In some embodiments, the FGF1 polypeptide composition for intranasal administration can be adapted for aerosolization and inhalation. The composition can be administered nasally via pressurized aerosol, aqueous pump spray or other standard methods known to those skilled in the art. Details on mode of intranasal administration and delivery devices are described in previous section.

Intranasal formulation containing FGF1 polypeptide can take the form of gels. Gels are three dimensional networks with a high viscosity containing the active molecule. Formulations comprising blending of Chitosan (CS), a β-(1-4)-linked D-glucosamine and N-acetyl-D-glucosamine co-molecule with a thermosensible poloxamer can result in formation of thermosetting gel which has a phase transition below the temperature of the nasal cavity (32° C. to 35° C.) and above room temperature. Therefore it can be administered as a liquid. Methods of formulation of the gel are taught in reference (54). These methods can be adapted for formulations with FGF1 polypeptide.

U.S. Pat. No. 5,756,483 incorporated as reference (51) teaches the use of formulation comprising cyclodextin and/or other saccharides and/or sugar alcohols for intranasal administration of apomorphine, the formulations of which can be adapted for intranasal administration of FGF1 polypeptide and are incorporated herein by reference.

FGF1 can also be administered using a gene therapy construct, e.g., as described in (55). Thus, in some embodiments, a pharmaceutical composition comprises an expression vector comprising a sequence encoding an FGF1 polypeptide.

In some cases, a polynucleotide encoding FGF1 can be introduced into a cell in vitro and the cell subsequently introduced into the subject's brain, e.g., into the intracerebroventricular space. The cells can be autologous to the subject. In some embodiments, an FGF1-encoding polynucleotide construct is introduced directly into cells in the subject in vivo.

Viral and non-viral-based gene transfer methods can be used to introduce nucleic acids encoding FGF1 polypeptides to cells or target tissues of the subject. Such methods can be used to administer nucleic acids encoding FGF1 polypeptides to cells in vitro. Alternatively, or in addition, such polynucleotides can be administered for in vivo or ex vivo gene therapy uses. Non-viral vector delivery systems include DNA plasmids, naked nucleic acid, and nucleic acid complexed with, for example, a liposome or other delivery vehicle. Viral vector delivery systems include both DNA and RNA viruses, and can have either episomal or integrated genomes after delivery to the cell. Gene therapy procedures are described, for example, in Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994).

Methods of non-viral delivery of nucleic acids encoding engineered polypeptides of the invention include lipofection, microinjection, biolistics, virosomes, liposomes, immunoliposomes, polycation or lipid:nucleic acid conjugates, naked DNA, artificial virions, and agent-enhanced uptake of DNA. Lipofection is described, e.g., in U.S. Pat. No. 5,049,386, U.S. Pat. No. 4,946,787; and U.S. Pat. No. 4,897,355, and lipofection reagents are sold commercially (e.g., Transfectam™ and Lipofectin™) Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

RNA or DNA viral based systems can be used to target the delivery of polynucleotides carried by the virus to specific cells in the body and deliver the polynucleotides to the nucleus. Viral vectors can be administered directly to patients (in vivo) or they can be used to transfect cells in vitro. In some cases, the transfected cells are administered to patients (ex vivo). Conventional viral based systems for the delivery of polypeptides of the invention could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene, and high transduction efficiencies.

FGF1 polypeptide can be synthesized using manual techniques or by automation. Automated synthesis can be achieved, for example, using Applied Biosystems 431A Peptide Synthesizer (Perkin Elmer). Alternatively, various fragments of the polypeptide (and any modified amino acids) can be chemically synthesized separately and then combined using chemical methods to produce the full length polypeptide. The sequence and mass of the polypeptides can be verified by GC mass spectroscopy. Once synthesized, the polypeptides can be modified, for example, by N-terminal acetyl- and C-terminal amide-groups as described above. Synthesized polypeptides can be further isolated by HPLC to a purity of at least about 80%, preferably 90%, and more preferably 95%.

### Combination Therapies

FGF1 polypeptide compositions can be administered, if necessary, with one or more additional agents for the treatment of elevated blood sugar or a metabolic disorder involving or characterized by abnormally high blood sugar or for treatment of one or more disorders or symptoms involved in or caused by metabolic syndrome or diabetes. The other therapeutic agent can be administered prior to, together with, after the administration of FGF1 polypeptide or on an entirely different therapeutic program. In some embodiments the method of treatment also comprises combined therapy with drugs e.g., small molecule or peptide, commonly used for treatment of metabolic disease and/or an anti-diabetic agent for e.g., insulin, an insulin sensitizer, an insulin secretagogue, an alpha-glucosidase inhibitor, an amylin agonist, a dipeptidyl-peptidase 4 (DPP-4) inhibitor, meglitinide, sulphonylurea, Metaformin, a glucagon-like peptide (GLP) agonist or a peroxisome proliferator-activated receptor (PPAR) agonist. PPAR-agonist for e.g., (PPAR)-gamma agonist such as Thiazolidinedione (TZD), aleglitazar, farglitazar, tesaglitazar, or muraglitazar. Exemplary TZD can be troglitazone, pioglitazone, rosiglitazone or rivoglitazone. Exemplary glucagon-like peptide agonist can be Liraglutide, Exenatide or Taspoglutide.

FGF1 can be administered in combination with insulin, e.g., in patients suffering from type 1 diabetes, hyperglycemia, abnormally elevated blood glucose levels (greater than or equal to 300 mg/dL) or insulopenia (decrease in levels of circulating insulin). In one embodiment, a combination therapy schedule can include pre-treatment with insulin prior to administration of an FGF1 polypeptide preparation. Where the inventors have found that FGF1 polypeptide administration is less effective when fasting blood glucose levels are greater 300 mg/dL prior to treatment, one approach for those who are severely hyperglycemic, e.g., as can occur in those with type 1 diabetes or those with severe type 2 diabetes symptoms is to administer one or more doses of insulin prior to FGF1 polypeptide administration to the brain. The insulin can transiently reduce blood glucose to less than or equal to 300 mg/dL and render the subject susceptible to effective treatment with the FGF1 polypeptide administered to the brain. Examples of insulin pre-treatment can be a single bolus injection, or injection or infusion of insulin over a longer period to affect blood glucose lowering to 300 mg/dL or less prior to FGF1 administration to the brain.

In some embodiments the therapeutic agent administered in combination with FGF1 can improve the efficacy of FGF1 treatment by acting in synergy with FGF1 treatment and/or complement FGF1 treatment to enhance the therapeutic outcome. The combination therapy therefore can allow for reduced dosages of FGF1 and other therapeutic agent compared to doses required for their effect individually and therefore can also potentially reduce any side effects that can occur with their individual treatment doses. As a non-limiting example, in combination therapy with FGF1 and TZD, the therapeutically effective dose of TZD can be reduced by about 10%, 20%, 30%, 40%, 50%, 60%, 70%, or about 80% compared to the typical dose of TZD used in treatment of type 2 diabetes. One skilled in the art can determine the best appropriate dose of the additional therapeutic agent after consideration of the patient, severity of disease, typical dose used in treatment and synergistic effect with FGF1 polypeptide.

The combination therapy with FGF1 and one or more therapeutic agent can be used to treat a disorder associated or related to metabolic disorder, a symptom thereof or a complication of metabolic disorder, e.g., for treating cardiovascular disease. Common conditions coexisting with type 2 diabetes including hypertension (Blood pressure ≧130/85 mm Hg) and dyslipidemia are risk factors for cardiovascular disease. Accordingly, a combination therapy can include an anti-hypertensive agent, e.g., a renin angiotensin aldosterone system antagonist (“RAAS antagonist”), an angiotensin converting enzyme (ACE) inhibitor, an angiotensin II receptor blocker (AT1 blocker), a diuretic, and/or an angiotensin II Receptor Blocker (ARB).

Patients with type 2 diabetes have an increased prevalence of lipid abnormalities, contributing to high risk cardiovascular disorders. A goal in the treatment of dyslipidemia is to lower LDL cholesterol to less than 100 mg/dL. Statins, e.g., Atorvastatin, Lovastatin and Simvastatin, among others, are commonly used for treatment of elevated LDL levels and can be administered orally as a combination therapy with an FGF1 polypeptide as described herein administered to the brain. An HDL level of <40 mg/dL in men or <50 mg/dL in women is considered a high risk of metabolic disorder. Niacin is commonly prescribed to increase HDL levels in such patients, and can be administered concurrently with FGF1 polypeptide as described herein.

Elevated triglyceride levels (e.g., greater than or equal to 150 mg/dL) or hypertriglyceridemia is a comorbidity of diabetes and an indicator for metabolic syndrome. Examples of drugs commonly used for treatment of high triglyceride levels include but are not limited to niacin, fibrates, and omega-3 fatty acids. Accordingly, in some embodiments, methods of treatment described herein can comprise a cholesterol- and/or triglyceride-lowering agent in combination with FGF1. Aspirin therapy is recommended in patients with type 1 and type 2 diabetes at risk of cardiovascular disease. Inflammation can also cause insulin resistance and diabetes complications. Therefore in some embodiments, methods of treatment described herein can comprise administration of an anti-inflammatory agent and/or anti-thrombotic agent in combination with FGF1. Non-limiting examples include, e.g., aspirin, IL-1 or IL-1 receptor antagonist, such as anakinra (KINERET®), rilonacept, or canakinumab, or an anti-TNFα antibody, such as infliximab (REMICADE®), golimumab (SIMPONI®), and/or adalimumab (HUMIRA®).

In some embodiments treatment with methods described herein can also benefit complications of diabetes. Examples of such complications include but are not limited to; (i) Diabetic nephropathy occurs in 20-40% of patients with diabetes. These patients also exhibit increased urinary albumin secretion (albuminuria). (2) Diabetic eye disease comprises a group of eye conditions that affect people with diabetes. These conditions include diabetic retinopathy, diabetic macular edema (DME), cataract, and glaucoma. Diabetic macular edema can be treated with Anti-VEGF injection therapy and corticosteroids. (3) Diabetic neuropathies are nerve disorders caused by diabetes e.g., distal symmetric polyneuropathy, diabetic autonomic neuropathy, gastrointestinal neuropathies, and genitourinary tract disturbances. (4) Diabetic foot ulcers, foot lesions and foot care. Amputation and foot ulceration, consequences of diabetic neuropathy are major causes of morbidity and disability in people with diabetes. (5) Depression, anxiety and other mental health symptoms are highly prevalent in patients with diabetes. (6) Obstructive sleep apnea occurrence is significantly higher with obesity. (7) Fatty liver disease e.g., nonalcoholic chronic liver disease and hepatic carcinoma are significantly associated with diabetes, higher BMI, waist circumference, triglycerides and fasting insulin and lower HDL cholesterol. (8) Cancer of the liver, pancreas, endometrium, colon/rectum, breast, and bladder are associated with type 2 diabetes. (9) Age-matched hip fracture risk is significantly increased in both type 1 and type 2 diabetes. (10) Low testosterone in men is observed in men with diabetes compared to men without the diabetes. (11) periodontal disease is more severe in patients with diabetes. (12) Celiac disease occurs at the rate of 8% in patients with diabetes compared to 1% in general population. (13) Thyroid disorder prevalence is high in patients with diabetes. (14) Cystic fibrosis related diabetes is the most common comorbidity in persons with cystic fibrosis (15) Tissue fibrosis e.g., kidney fibrosis occurs as a result of chronic hyperglycemia. In some embodiments, the method of treatment also comprises an anti-fibrotic agent in combination with FGF1. Therapeutic agents commonly used for treatment of above mentioned complications can be used in combination with FGF1.

### Dosage and Administration

An effective therapeutic dosage administered to the patient will depend, among other factors, upon the subject's history, age, condition and sex, as well as the severity and type of the medical condition in the subject, and the administration of other pharmaceutically active agents. Furthermore, therapeutically effective amounts will vary, as recognized by those skilled in the art, depending on the specific disease treated, the route of administration, the excipient selected, frequency of administration and the possibility of combination therapy. Thus, a single specific dose of FGF1 polypeptide suitable for all subjects is not likely to be practical. Nonetheless, one of skill in the art can arrive at an effective dose without undue experimentation using principles known in the art and the guidance provided herein.

Of primary importance is the understanding that the dose of FGF1 polypeptide administered to the brain will be less than half of that sufficient to provide a transient reduction in blood sugar when the FGF polypeptide is administered systemically. Indeed, the level can be less than half the level required for transient systemic effect, less than or equal to 40%, less than or equal to 30%, less than or equal to 25%, less than or equal to 20%, less than or equal to 15%, less than or equal to 10% or lower relative to the amount required for a transient blood glucose lowering effect upon systemic administration. By “transient blood glucose lowering effect” in this context is meant a reduction of blood glucose levels to within the normal range when administered a single dose of the agent, wherein the levels remain in the normal range for less than 3 days. One approach, then for identifying an effective dose of an FGF1 polypeptide for administration to the brain is to first administer successively escalating amounts of FGF1 polypeptide composition systemically, e.g., intravenously, and monitor for a reduction in elevated blood glucose to within the normal levels. Once the level effective to achieve a transient decrease in blood glucose level when administered systemically is determined, a dose less than half of that, and preferably less than or equal to 40%, less than or equal to 30%, less than or equal to 25%, less than or equal to 20%, less than or equal to 15%, less than or equal to 10% or lower can be selected for effective administration to the brain as a unit dose formulation. One of skill in the art will be able to adjust the dose to account for differing molar amounts when, e.g., a smaller functional fragment of FGF1 or a conjugate is administered.

As noted above, administration to the brain includes administration via intracerebroventricular, intracranial, intracerebellar, intracelial, or intrathecal administration routes and encompasses, in some embodiments, intranasal delivery. While each of these routes can deliver administered polypeptide to the brain, the intranasal route differs from the others in that uptake is generally less efficient. Compositions and methods that aim to maximize uptake via the intranasal route are discussed herein and known in the art; however, in general, unit dosages for intranasal delivery will need to be considerably higher, generally at least 10-fold higher, than for the other routes of administration to the brain. Thus, where, for example, a unit dose for administration via the intracerebroventricular route may be 100 μg of FGF1 polypeptide, a unit dose of 1000 μg or more would be indicated for the intranasal route. It should be understood, then, that where a unit dose or unit dose formulation is referred to herein for administration to the brain, the unit dose or unit dose formulation for intranasal administration to the brain will be at least 10 times that recited. To be clear, for the intracerebroventricular, intracranial, intracerebellar, intracelial, or intrathecal administration routes, the values are as recited.

Unit dose preparations of FGF1 polypeptide composition formulated for administration to the brain and effective to establish prolonged maintenance of blood glucose levels within the normal range with a single administration can be prepared with varying amounts of active FGF1 polypeptide. For example, a formulation including 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg or more of FGF1 polypeptide, e.g., full length human FGF1 polypeptide, can be prepared in a unit dose form for administration to the brain, and can include excipients or other agents that promote uptake within the brain as known in the art or as described herein. Alternatively, a unit dose can include an amount effective, upon administration to the brain, to reduce an abnormally high blood glucose level for a prolonged period as that term is used herein and can include, for example, 250 μg or less, 240 μg or less, 230 μg or less, 220 μg or less, 210 μg or less, 200 μg or less, 190 μg or less, 180 μg or less, 170 μg or less, 160 μg or less, 150 μg or less, 140 μg or less, 130 μg or less, 120 μg or less, 110 μg or less, 100 μg or less, 90 μg or less, 80 μg or less, 70 μg or less, 60 μg or less, 50 μg or less, 40 μg or less, or even 30 μg or less of an FGF1 polypeptide, e.g., full length human FGF1 polypeptide or an equivalent molar amount of a fragment or derivative thereof. As noted above, where the unit dose is formulated for administration to the brain via the intranasal route, the unit dose will be at least 10 times the number recited above in this paragraph. Where an FGF1 polypeptide is a functional fragment or derivative thereof that retains blood glucose reducing or normalizing activity of FGF1 but is different in size, the amount of the FGF1 polypeptide in the unit dose preparation can be adjusted by one of skill in the art to maintain an equivalent molar amount of the differently sized polypeptide.

In some embodiments the preferred routes of administration can be intracerebroventricular, intranasal, intracranial, intracerebellar, intracelial, or intrathecal. In some embodiments the treatment can be a single administration of a therapeutically effective unit dose of FGF1 polypeptide containing composition. In some embodiments the treatment can be administered once weekly, biweekly, monthly, bimonthly, every 3 months, 4 months, 5 months, 6 months or more, or, for example, once per year as needed to maintain therapeutic effect. The pharmaceutical preparation for the methods of treatment described herein can be packaged as physically discrete units suitable as a unit dosage.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the invention. Further, all patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Embodiments of various aspects described herein can be defined in any of the following numbered paragraphs:
1. A pharmaceutical composition comprising a unit dose of Fibroblast Growth Factor 1 (FGF1) polypeptide preparation comprising a pharmaceutically acceptable carrier and formulated for administration to the brain.
2. The composition of paragraph 1, wherein the composition is formulated for administration via an intracerebroventricular, intranasal, intracranial, intracelial, intracerebellar, or intrathecal administration route.
3. The composition of paragraph 2, wherein the composition is formulated for administration via an intranasal route and further comprises a ganglioside and/or a phosphotidylserine.
4. The composition of paragraph 2, wherein the composition is formulated for administration via an intranasal route and further comprises saccharides selected from the group of cyclodextrins, disaccharides, polysaccharides, and combinations thereof.
5. The pharmaceutical composition of any one of paragraphs 1-4, further comprising another FGF family member polypeptide.
6. The pharmaceutical composition of any one of paragraphs 1-5, wherein the FGF1 polypeptide is a human FGF1 polypeptide.
7. The pharmaceutical composition of any one of paragraphs 1-6, wherein the FGF1 polypeptide has at least 95% amino acid sequence identity to SEQ ID NO:1 and retains at least 80% of the biological activity of human FGF1 of SEQ ID NO: 1.
8. The pharmaceutical composition of paragraphs 7, wherein, the FGF1 polypeptide is a human recombinant polypeptide.
9. The pharmaceutical composition of any one of paragraphs 7-8, wherein the FGF1 polypeptide comprises amino acids 1-155 of SEQ ID NO: 1.
10. The pharmaceutical composition of any one of paragraphs 7-8, wherein the FGF1 polypeptide comprises at least amino acids 25-155 of SEQ ID NO: 1.
11. The pharmaceutical composition of any one of paragraphs 1-10, wherein the composition is contained in a delivery device selected from the group consisting of a syringe, a blunt tip syringe, a catheter, an inhaler, a nebulizer, a nasal spray pump, a nasal irrigation pump or nasal lavage pump, and an implantable pump.
12. The pharmaceutical composition of any one of paragraphs 1-11, wherein the FGF1 polypeptide is formulated with a lipophilic molecular group.
13. The pharmaceutical composition of any one of paragraphs 1-12, wherein the FGF1 polypeptide is encapsulated in a liposome or a nanoparticle.
14. The pharmaceutical composition of any one of paragraphs 1-13 wherein the FGF1 polypeptide is fused to a carrier polypeptide.
15. The pharmaceutical composition of paragraph 1-14 wherein unit dose of Fibroblast Growth Factor 1 (FGF1) polypeptide is less than 50% of the unit dose required to treat diabetes via systemic administration.
16. The pharmaceutical composition of any one of paragraphs 1-15 wherein the unit dose comprises less than about 100 μg of the FGF1 polypeptide.
17. A pharmaceutical composition comprising a unit dose of a Fibroblast Growth Factor 1 (FGF1) polypeptide preparation comprising a pharmaceutically acceptable carrier and formulated for administration to the brain, wherein the unit dose of FGF1 polypeptide is 100 μg or less.
18. A pharmaceutical composition comprising a unit dose of a Fibroblast Growth Factor 1 (FGF1) polypeptide preparation comprising a pharmaceutically acceptable carrier and formulated for administration to the brain, wherein the unit dose of FGF1 polypeptide is less than half of the unit dose required to normalize blood glucose levels when the FGF1 polypeptide is administered systemically.
19. A pharmaceutical composition formulated for administering a FGF1 polypeptide to the brain, the composition comprising an FGF1 polypeptide and heparin.
20. A pharmaceutical composition formulated for administering a FGF1 polypeptide to the brain, the composition comprising an FGF1 polypeptide and heparan sulfate.
21. A method of treating a metabolic disorder in a subject, the method comprising administering a unit dose of a pharmaceutical composition comprising a Fibroblast Growth Factor 1 (FGF1) polypeptide preparation of paragraph 1 to the brain of a subject having a metabolic disorder, wherein the metabolic disorder is treated.
22. The method of paragraph 21, wherein the administration is intracerebroventricular administration, intranasal administration, intracranial administration, intracerebellar administration, intracelial administration, or intrathecal administration.
23. The method of paragraph 21 or 22, wherein the metabolic disorder is a disorder characterized by or involving abnormally elevated blood glucose levels.
24. The method of paragraph 23 wherein the metabolic disorder is selected from the group consisting of type 2 diabetes, gestational diabetes, drug-induced diabetes, high blood glucose, insulin resistance and metabolic syndrome.
25. The method of any one of paragraphs 21-24, further comprising the step, prior to the administering step, of diagnosing the patient as having a metabolic disorder.
26. The method of any one of paragraphs 21-25, wherein prior to administration of the pharmaceutical composition the subject has a blood glucose level above the normal range, and wherein administration of the composition lowers blood glucose level to within the normal range.
27. The method of any one of paragraphs 21-26, wherein the administration of the pharmaceutical composition does not result in hypoglycemia.
28. The method of any one of paragraphs 21-27, wherein the administration does not result in a sustained loss of body weight and/or reduced food intake.
29. The method of any one of paragraphs 21-28, wherein the unit dose of the pharmaceutical composition required to normalize blood glucose level is less than 50% of the unit dose required to normalize blood glucose when a FGF1 polypeptide is administered systemically.
30. The method of any one of paragraphs 21-29, wherein the unit dose administered comprises 100 μg or less of the FGF1 polypeptide.
31. The method of any one of paragraphs 21-30, wherein a single unit dose of the administered pharmaceutical composition normalizes blood glucose level in the subject for at least one week.
32. The method of any one of paragraphs 21-30, wherein the pharmaceutical composition is administered weekly.
33. The method of any one of paragraphs 21-32, further comprising administering another FGF family member polypeptide to the subject.
34. The method of any one of paragraphs 21-33, further comprising administering one or more agents selected from the group consisting of an anti-inflammatory agent, an anti-fibrotic agent, an anti-hypertensive agent, an anti-diabetic agent, a triglyceride lowering agent, and a cholesterol lowering agent to the subject.
35. The method of paragraph 34, wherein the anti-diabetic agent is selected from the group consisting of insulin, an insulin sensitizer, an insulin secretagogue, an alpha-glucosidase inhibitor, an amylin agonist, a dipeptidyl-peptidase 4 (DPP-4) inhibitor, meglitinide, sulphonylurea, Metaformin, a glucagon-like peptide (GLP) agonist or a peroxisome proliferator-activated receptor (PPAR)-gamma agonist.
36. The method of paragraph 35, wherein the PPAR-gamma agonist is a Thiazolidinedione (TZD), aleglitazar, farglitazar, tesaglitazar, or muraglitazar.
37. The method of paragraph 36, wherein the TZD is troglitazone, pioglitazone, rosiglitazone or rivoglitazone.
38. The method of paragraph 35, wherein the Glucagon-like peptide (GLP) agonist is Liraglutide, Exenatide or Taspoglutide.
39. The method of any one of paragraphs 21-38, wherein the subject is a mammal.
40. The method of any one of paragraphs 21-39, wherein the subject is a human.
41. The method of any one of paragraphs 21-40, wherein the blood glucose levels are lowered to normal range in 6 hours or less after a single administration of the pharmaceutical composition.
42. The method of any one of paragraphs 21-40, wherein the blood glucose levels are normalized in 24 hours or less after a single administration the pharmaceutical composition.
43. The method of any one of paragraphs 21-40, wherein the blood glucose levels are normalized in 1 week or less after a single administration of the pharmaceutical composition.
44. The method of any one of paragraphs 21-43, wherein, the FGF1 polypeptide comprised by the pharmaceutical composition is a human FGF1 polypeptide.
45. The method of any one of paragraphs 21-44, wherein the FGF1 polypeptide has at least 95% amino acid sequence identity to SEQ ID NO:1 and retains at least 80% of the biological activity of human FGF1 of SEQ ID NO: 1.
46. The method of any one of paragraphs 21-45, wherein the FGF1 polypeptide is a human recombinant polypeptide.
47. The method of any one of paragraphs 21-46, wherein the FGF1 polypeptide comprises amino acids 1-155 of SEQ ID NO: 1.
48. The method of any one of paragraphs 21-47, wherein the FGF1 polypeptide comprises at least amino acids 25-155 of SEQ ID NO: 1.
49. The method of any one of paragraphs 21-48, wherein the FGF1 polypeptide preparation comprises a carrier peptide or lipophilic molecular group and/or is encapsulated in a liposome or a nanoparticle.
50. A method of treating diabetes in a subject, the method comprising administering a single unit dose of a pharmaceutical composition comprising a Fibroblast Growth Factor 1 (FGF1) polypeptide preparation to the brain of a subject having diabetes, wherein blood glucose levels are normalized for at least 18 weeks.
51. A method of treating elevated blood glucose levels in a subject in need thereof, comprising administering an FGF1 polypeptide to the brain of the subject, whereby blood glucose levels are lowered to a normal range.
52. A method to induce sustained diabetes remission in a subject in need thereof, comprising administering an FGF1 polypeptide to the brain of the subject.
53. A method to treat high blood glucose levels in a subject in need thereof, comprising administering a therapeutically effective amount of an FGFR binding protein to the brain of the subject to normalize the blood glucose levels to normal range, wherein the FGFR is selected from the group, FGFR1, FGFR2, FGFR3, FGFR4 or a combination thereof.
54. The method of paragraph 53 wherein the FGFR binding protein is a FGF1 polypeptide.
55. A method of treating diabetes in a subject, comprising administering to a subject having diabetes a composition of any one of paragraphs 1-20.
56. A pharmaceutical composition comprising a unit dose of a FGF1 polypeptide preparation for use in the treatment of a metabolic disorder, wherein the composition is formulated for delivery to the brain, wherein the unit dose of a FGF1 polypeptide is 100 μg or less.
57. A pharmaceutical composition comprising a unit dose of a FGF1 polypeptide preparation for use in the treatment of a metabolic disorder, wherein the composition is formulated for delivery to the brain, wherein the unit dose of a FGF1 polypeptide is less than 50% of the unit dose required to normalize blood glucose when a FGF1 polypeptide is administered systemically.
58. The pharmaceutical composition for use of paragraph 56 or 57, wherein the metabolic disorder is selected from the group consisting of type 2 diabetes, gestational diabetes, drug-induced diabetes, high blood glucose, insulin resistance, metabolic syndrome.
59. The pharmaceutical composition for use of paragraph 57 or 58, wherein the composition is formulated for administration via an intracerebroventricular, intranasal, intracranial, intracelial, intracerebellar, or intrathecal administration route.
60. The pharmaceutical composition for use of any one of paragraphs 57-59, further comprising another FGF family member polypeptide.
61. The pharmaceutical composition for use of any one of paragraphs 57-60, wherein the FGF1 polypeptide is a human FGF1 polypeptide.
62. The pharmaceutical composition for use of any one of paragraphs 57-61, wherein the FGF1 polypeptide has at least 95% amino acid sequence identity to SEQ ID NO:1 and retains at least 80% of the biological activity of human FGF1 of SEQ ID NO: 1.
63. The pharmaceutical composition for use of any one of paragraphs 57-62, wherein, the FGF1 polypeptide is a human recombinant polypeptide.
64. The pharmaceutical composition for use of any one of paragraphs 57-63, wherein the FGF1 polypeptide comprises amino acids 1-155 of SEQ ID NO: 1.
65. The pharmaceutical composition for use of any one of paragraphs 57-63, wherein the FGF1 polypeptide comprises at least amino acids 25-155 of SEQ ID NO: 1.
66. The pharmaceutical composition for use of any one of paragraphs 57-65, wherein the composition is contained in a delivery device selected from the group consisting of a syringe, a blunt tip syringe, a catheter, an inhaler, a nebulizer, a nasal spray pump, a nasal irrigation pump or nasal lavage pump, and an implantable pump.
67. The pharmaceutical composition for use of any one of paragraphs 57-66, wherein the FGF1 polypeptide is formulated with a lipophilic molecular group.
68. The pharmaceutical composition for use of any one of paragraphs 57-67, wherein the FGF1 polypeptide is encapsulated in a liposome or a nanoparticle.
69. The pharmaceutical composition for use of any one of paragraphs 57-68, wherein the FGF1 polypeptide is fused to a carrier polypeptide.
70. A pharmaceutical composition formulated for intranasal administration to a subject in need thereof, comprising a unit dose of a Fibroblast Growth Factor 1 (FGF1) polypeptide preparation in combination with a ganglioside and/or a phosphotidylserine, wherein the unit dose of a Fibroblast Growth Factor 1 (FGF1) polypeptide is 100 μg or less.
71. A pharmaceutical composition formulated for intranasal administration to a subject in need thereof, comprising a unit dose of a Fibroblast Growth Factor 1 (FGF1) polypeptide preparation in combination with a ganglioside and/or a phosphotidylserine, wherein the unit dose of a Fibroblast Growth Factor 1 (FGF1) polypeptide is less than 50% of the unit dose required to normalize blood glucose when an FGF1 polypeptide is administered systemically.
72. A pharmaceutical composition formulated for intranasal administration to a subject in need thereof, comprising a unit dose of a Fibroblast Growth Factor 1 (FGF1) polypeptide preparation in combination with a saccharide selected from the group consisting of cyclodextrins, disaccharides, polysaccharides, and combinations thereof, and wherein the unit dose of a Fibroblast Growth Factor 1 (FGF1) polypeptide is 100 μs or less.
73. A pharmaceutical composition formulated for intranasal administration to a subject in need thereof, comprising a unit dose of a Fibroblast Growth Factor 1 (FGF1) polypeptide preparation in combination with a saccharide selected from the group consisting of cyclodextrins, disaccharides, polysaccharides, and combinations thereof, and wherein the unit dose of a Fibroblast Growth Factor 1 (FGF1) polypeptide is less than 50% of the unit dose required to normalize blood glucose when an FGF1 polypeptide is administered systemically.
74. A method of treating diabetes in a subject who has a blood glucose level greater than or equal to 300 mg/dL prior to treatment, the method comprising administering insulin and administering a single dose FGF1 polypeptide preparation to the brain, wherein blood glucose levels are normalized for at least 1 week.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The technology described herein is further illustrated by the following examples which in no way should be construed as being further limiting.

### Example 1

### Methods

Animals.

Adult, male ob/ob (B6.Cg-Lepob/J), ob/ob (BTBR.Cg-Lepob/WiscJ), db/db (B6.BKS(D)-Leprdb/J), C57BL/6J (WT) mice (Jackson Laboratories) and ZDF rats (ZDF-Leprfa/Crl; Charles River) were housed individually under specific pathogen-free conditions in a temperature-controlled room with a 12:12 h light:dark cycle. Mice were provided with ad-libitum (ad-lib) access to water and either standard laboratory chow (LabDiet, St. Louis, Mo.) or a 60% high-fat diet (HFD; D12492, Research Diets), unless otherwise stated. ZDF rats were provided with ad-lib access to water and Purina 5008 diet (Animal Specialties, Inc., Hubbard, Oreg.). All procedures were performed in accordance with NIH guidelines for the care and use of animals and were approved by the Institutional Animal Care and Use Committee at either the University of Washington (Seattle, Wash.) or Vanderbilt University (Nashville, Tenn.).

Surgery.

Cannulation of the lateral ventricle (LV; 26-ga, Plastics One, Roanoke, Va.) was performed under isoflurane anesthesia using stereotaxic coordinates based on the brain atlas (For mice, −0.7 mm posterior to bregma; 1.3 mm lateral, and 1.3 mm below the skull surface; and for rats, −0.8 mm posterior to bregma; 1.5 mm lateral, and 2.6 mm below the skull surface). For measurement of basal glucose turnover followed by the Frequently sampled insulin-modified intravenous glucose tolerance tests (FSIGT), adult male ob/ob (B6) mice underwent LV cannulation and catheterization of both the carotid artery and the internal jugular vein during the same surgical session. Animals received buprenorphine hydrochloride (Reckitt Benckiser Pharmaceuticals Inc., Richmond, Va.) at the completion of the surgery and were allowed to recover for at least 7 d prior to study while food intake and body weight were recorded.

DIO WT-STZ Mice.

After placement on a HFD for 3 mo to induce diet-induced obesity (DIO), WT mice underwent cannulation of the LV and 7 d later received either three consecutive daily subcutaneous (sc) injections of streptozotocin (STZ; Sigma-Aldrich, MO) at a low dose (40 mg/kg body weight) (DIO-LD STZ) to induce moderate hyperglycemia (˜150-200 mg/dl), or a single intraperitoneal (ip) injection of high-dose STZ of 100 mg/kg body weight (DIO-HD STZ) to induce more severe hyperglycemia. Measures of blood glucose (BG) levels, food intake, and body weight were recorded throughout the study.

Intracerebroventricular (icv) Injections.

Rodents were monitored for several days to ensure that mean BG values were matched between study groups prior to icv injection. Recombinant mouse FGF1 (mFGF1; Prospec, NJ) or recombinant human FGF1 (hFGF1; Novo Nordisk) were dissolved in sterile water or phosphate-buffered saline (PBS), respectively, at a concentration of 1.5 μg/μl and injected over 60 s into the LV in a final volume of 2 μl using a (33-ga) needle extending 0.8 mm beyond the tip of the icv cannula. Recombinant human FGF19 (Phoenix Pharmaceuticals) was dissolved in 0.9% normal saline at a concentration of 2 μg/μl and was administered via the LV as described in (ref 8). Recombinant rat FGF1 (rFGF1; Prospec, NJ) was dissolved in sterile water at a concentration of 1 μg/μl and injected over 60 s into the LV in a final volume of 3 μl using a (33-ga) needle extending 1 mm beyond the tip of the icv cannula.

Subcutaneous (sc) Injections.

Recombinant mouse FGF1 (mFGF1; Prospec, NJ) was dissolved in sterile water at a concentration of 1.5 μg/μl and administered sc in a final volume of 50 μl of vehicle (Veh) solution (0.9% normal saline).

Intraperitoneal Glucose Tolerance Testing (ipGTT).

ipGTTs were conducted in 6 h-fasted animals by measuring BG levels at t=0, 15, 30, 60, 90, and 120 min from a tail capillary blood sample using a hand-held glucometer (Accu-Chek FreeStyle Lite) following an ip injection of glucose (30% dextrose) at a dose of either 0.5 or 2 g/kg body weight, depending on basal glycemia.

Body Composition Analysis.

Total body fat mass was measured using quantitative magnetic resonance spectroscopy (EchoMRI 3-in-1 Animal Tissue Composition Analyzer; Echo Medical Systems) available through the Energy Balance and Glucose Metabolism Core of the Nutrition Obesity Research Center at the University of Washington.

Basal Glucose Turnover.

Basal glucose turnover analysis as described in (ref 56) was performed in 5 h-fasted ob/ob (B6) mice 7 d after receiving icv injection of either mFGF1 (3 μg) or vehicle (Veh; 0.9% normal saline). At t=−90 min, a continuous intravenous (iv) infusion of [3-3H] glucose was commenced (10 μCi bolus+0.05 μCi·min-1). Blood samples were taken at t=−10 and 0 min to calculate the basal glucose turnover rate (GTR), which at steady state is equal to the rates of both glucose production and glucose disposal, and the peripheral glucose clearance rate (calculated as the glucose disposal rate divided by the plasma glucose concentration as described in (ref 57).

Frequently Sampled Intravenous Glucose Tolerance Test (FSIGT).

Following the basal glucose turnover study, the same cohort of ob/ob (B6) mice was subjected to an FSIGT. Blood sampling was performed via an arterial catheter in unrestrained, conscious animals. A continuous infusion of saline-washed erythrocytes was commenced at t=0 min to prevent a >5% fall in hematocrit. Baseline fasting blood samples were drawn at −10 and 0 min. Based on a published protocol,28 a bolus of 50% dextrose (0.75 g/kg body weight) was injected iv over a period of 15 s at t=0 min. Blood (20 μl) was sampled both for measurement of glucose using a hand-held glucometer (Accu-Chek) and for subsequent assay of plasma insulin and lactate levels at time points 1, 2, 4, 8, 12, 16, 20, 30 and 60 min after the glucose injection. Additional samples were obtained for blood glucose measurement at 3, 5, 6, 10, 14, 18, 25, 40 and 50 min using a hand-held glucometer.

Minimal Model Analysis and Calculations.

The plasma insulin and blood glucose profiles generated from the FSIGTs were analyzed using MinMod software to quantify insulin-independent glucose disposal (SG) and insulin sensitivity (SI), as described in (reference 58). From the FSIGT, insulin secretion was quantified as the acute insulin response to glucose (AIRg), a measure of islet β-cell function in response to a glucose load, based on plasma insulin values between t=0-4 min.

Peripheral Administration of Insulin Receptor Antagonist.

WT mice fed a HFD for 3 mo underwent LV cannulation. Following a 1 wk recovery, mice underwent sc implantation of an osmotic micropump (Alzet, Durect Corp., CA) loaded with the high affinity insulin receptor antagonist S961 (dissolved in PBS; Novo Nordisk) at a dose designed to continuously infuse the drug at a rate of 29 nmol/wk for 2 wk. On Day 2 following micropump implantation, the mice received a single icv injection of either mFGF1 (3 μg) or Veh (0.9% normal saline). Daily BG levels, food intake and body weight were recorded throughout the study.

Plasma and Tissue Analysis.

Blood samples were collected into EDTA-treated tubes for measurement of plasma hormones and metabolites. Whole blood was centrifuged and plasma removed for subsequent measurement of plasma immunoreactive insulin [either by ELISA (Crystal Chem, Inc., IL) or by a radioimmunoassay kit from Millipore (Billerica, Mass.; performed by the Vanderbilt Diabetes Center Hormone Assay & Analytical Services Core)], and for measurement of glucagon and corticosterone levels by ELISA (Mercodia, NC; and ALPCO Diagnostics, NH). Plasma lactate levels were determined using a GM9D glucose direct analyzer (Analox Instruments). Plasma lipids were measured with enzymatic colorimetric assays using the following kits: Triglyceride (TG) and total cholesterol (Chol) from Raichem (San Diego, Calif.); non-esterified free fatty acid (NEFA) from Wako Diagnostics (Richmond, Va.). Liver glycogen levels were determined using a colorimetric assay (Biovision) and were normalized to grams wet weight.

RT-PCR.

Total RNA was extracted from liver and brown adipose tissue (BAT) using TriReagent (Sigma-Aldrich) and NucleoSpin RNA (Fischer Scientific). Levels of specific transcripts were quantified by real-time PCR (ABI Prism 7900 HT; Applied Biosystems) using SYBR Green (Applied Biosystems) and the following specific primers: GCK (forward-CAAGCTGCACCCGAGCTT; (SEQ ID NO:95), reverse-TGATTCGATGAAGGTGATTTCG; (SEQ ID NO:96), L-PK (forward-TGATGATTGGACGCTGCAA; (SEQ ID NO:97), reverse-CATTGGCCACATCGCTTG; (SEQ ID NO:98), GS (forward-ACCAAGGCCAAAACGACAG; (SEQ ID NO:99), reverse-GGGCTCACATTGTTCTACTTGA; (SEQIDNO:100), PEPCK (forward-GGCGGAGCATATGCTGATCC; (SEQ ID NO:101); reverse-CCACAGGCACTAGGGAAGGC; (SEQ ID NO:102), G6Pase (forward-TCAACCTCGTCTTCAAGTGGATT; (SEQ ID NO:103), reverse-CTGCTTTATTATAGGCACGGAGCT; (SEQ ID NO:104), and UCP-1 (forward-ACTGCCACACCTCCAGTCATT; (SEQ ID NO:105), reverse-CTTTGCCTCACTCAGGATTGG; (SEQ ID NO:106). Results were normalized to the housekeeping gene 18s (forward-CGGACAGGATTGACAGATTG; (SEQ ID NO:107), reverse-CAAATCGCTCCACCAACTAA (SEQ ID NO:108) to correct for internal variances. For comparative analysis, RNA ratios of the treatment group were normalized to the icv Veh control group. The sequences of the primers are described by the following SEQ ID submitted herewith.

Statistical Analysis and General Methods.

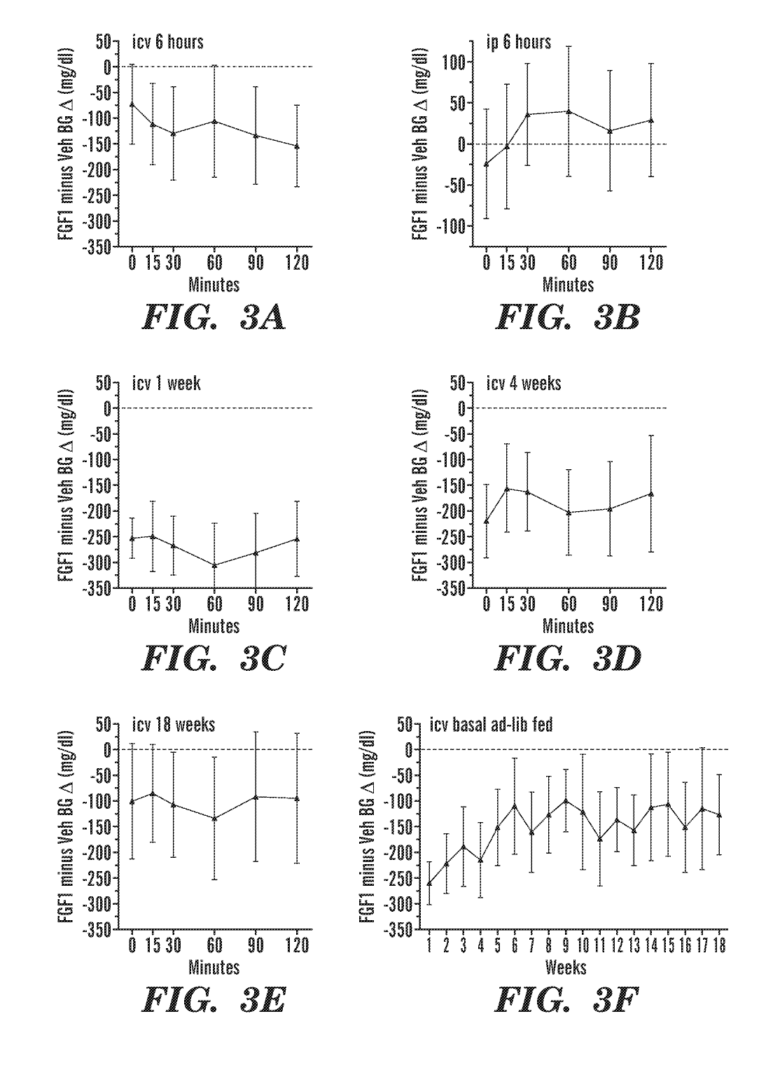

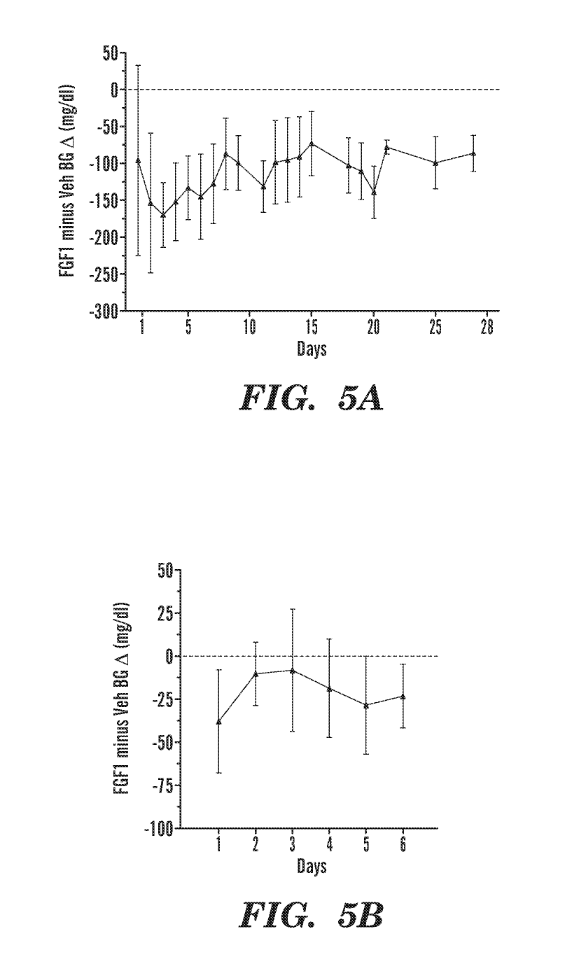

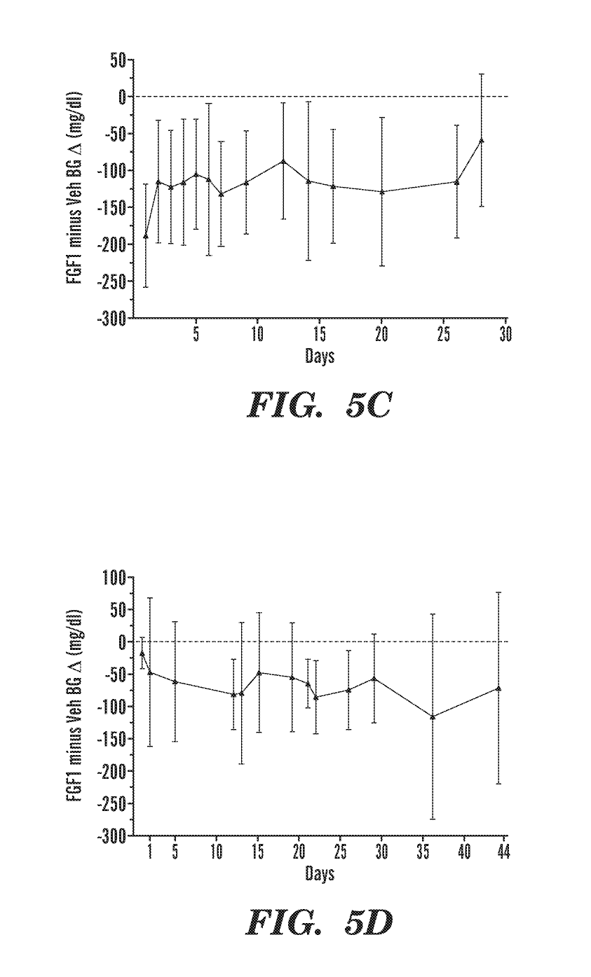

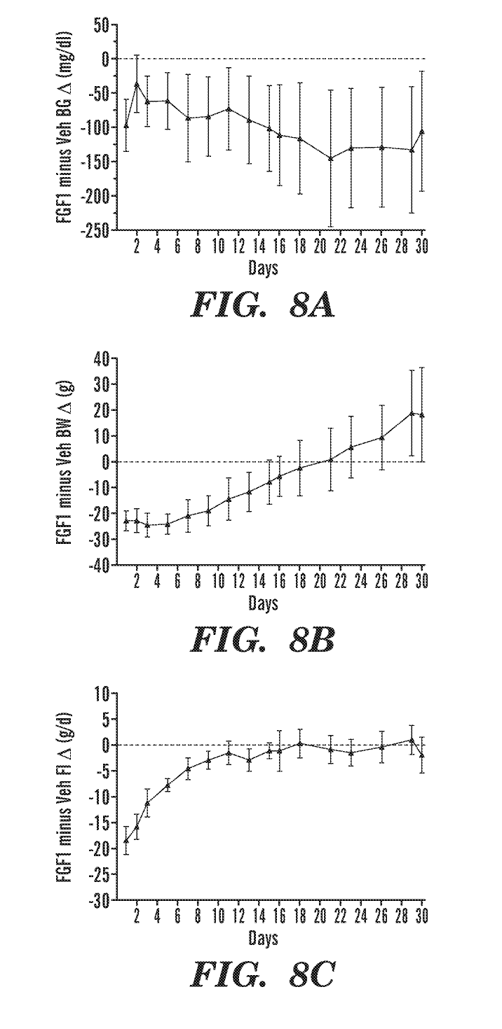

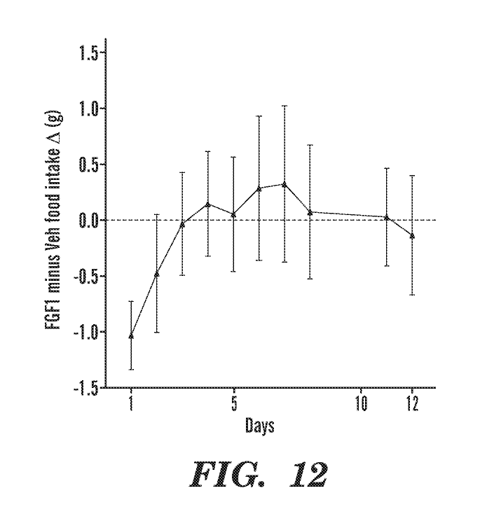

For each study, groups receiving icv Veh vs. FGF1 were matched for age, body weight and BG levels. Sample sizes of 6-8/group were predicated on detecting with ˜80% power a BG group difference of 100 mg/dl assuming a within group standard deviation of 55 mg/dl. Group by time mixed factorial designs were analyzed using linear mixed model analysis (SPSS v. 23, IBM Corp., Somers, N.Y.) and mixed factorial analyses (GraphPad software, La Jolla, Calif.). Basic pairwise comparisons were by independent samples t-tests with Satterthwaite adjustment for unequal variances where indicated by significant Levene's tests. Within time-point pairwise assessments of group differences were rendered in terms of 95% confidence intervals to convey effect sizes and their patterns over time (FIGS. 3, 5, 8 and 12). A two-sample unpaired Student's t-test was used for two-group comparisons and a one-way ANOVA was performed for three-group comparisons. Animals were not excluded from the studies unless otherwise indicated and the investigators were not blinded to study conditions. Alpha was set at P<0.05, 2-tail.

### Example 2

Time Course of the Glycemic Response to icv FGF1 Administration in Ob/Ob Mice.

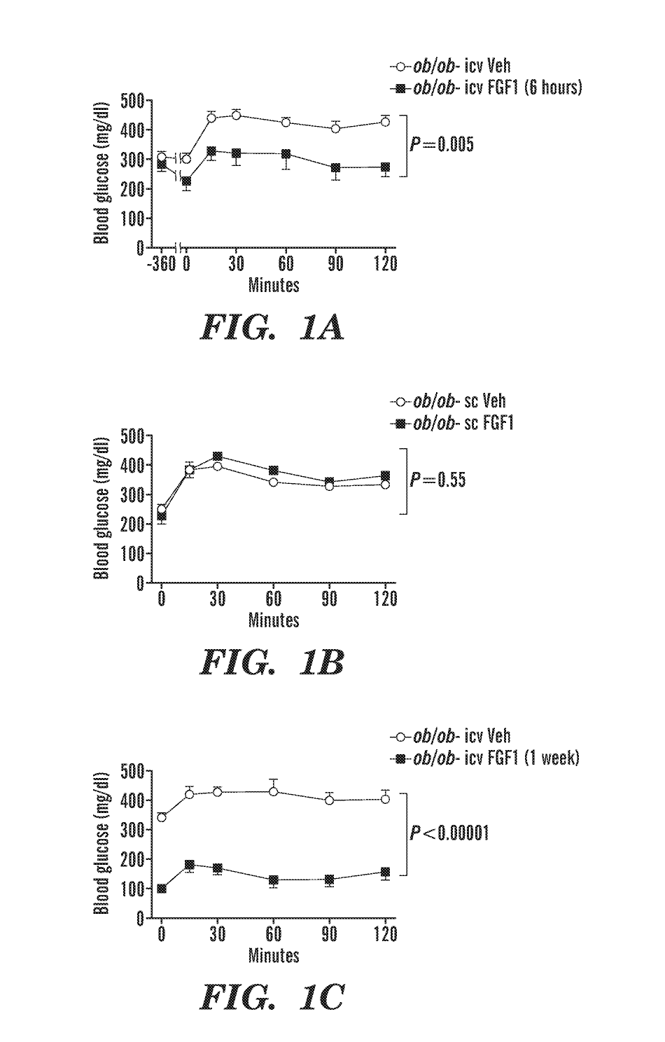

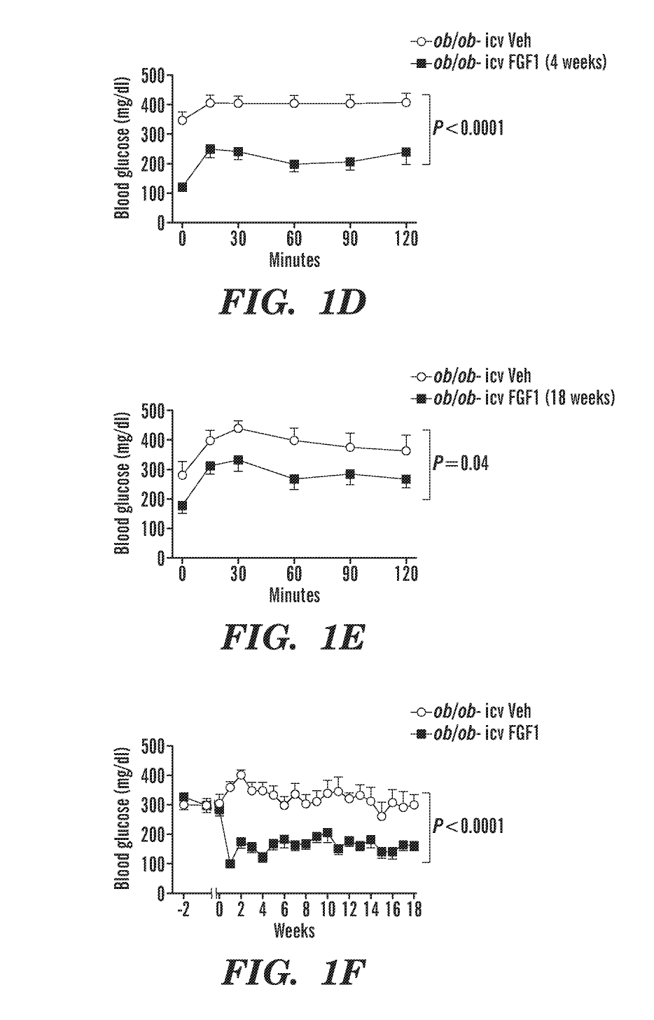

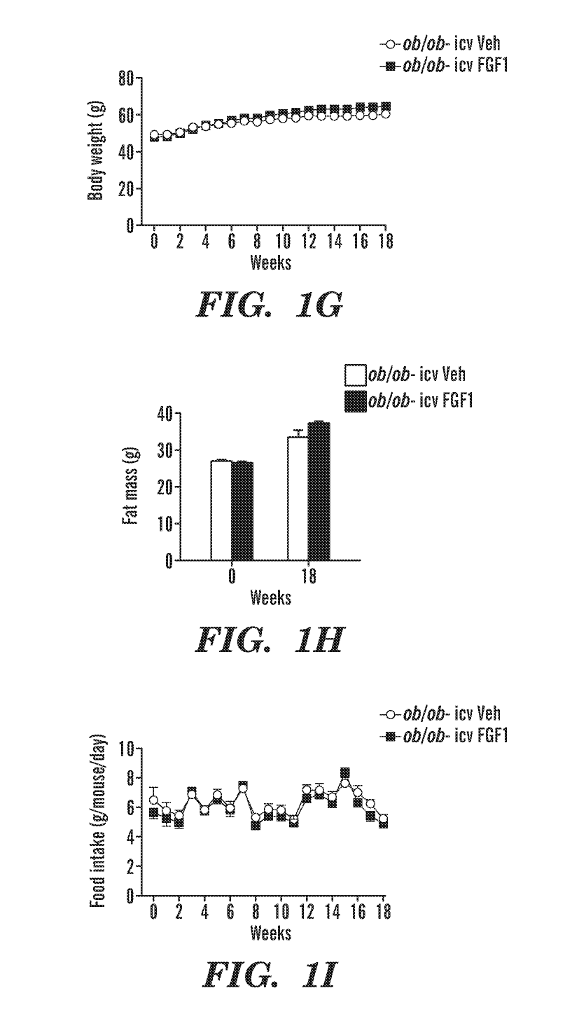

As a first demonstration that prolonged glucose lowering is achievable through activation of brain FGFRs, diabetic ob/ob mice received a single icv injection of recombinant murine FGF1 (mFGF1) at a dose (3 μg), which is 10-fold below that needed for systemic efficacy. Six hours later, a ˜25% decline of fasting BG levels was observed as shown in FIG. 1a (p<0.05). This effect cannot be explained by reduced food intake, since food was not available during this time, or by leakage from brain to periphery, since subcutaneous (sc) administration of FGF1 at the same dose was without effect (FIG. 1b).

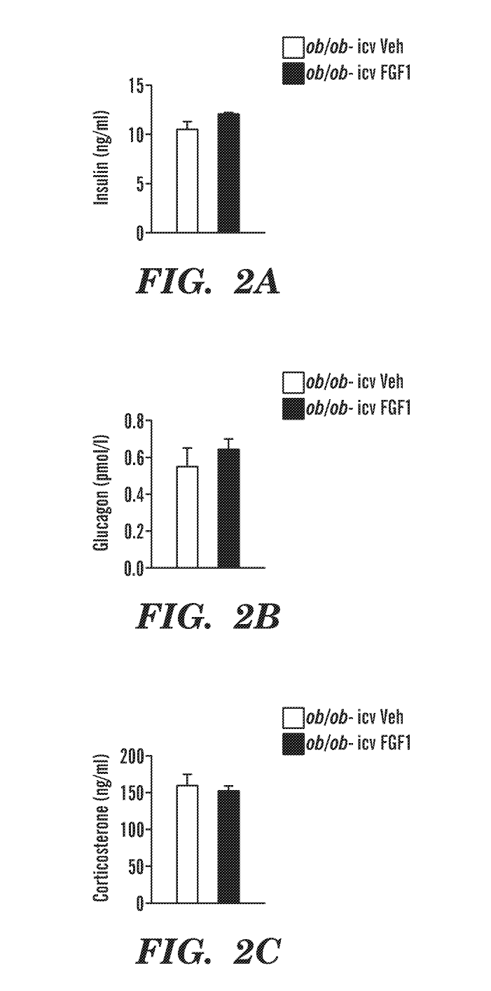

Remarkably, the glucose-lowering effect of a single icv injection of mFGF1 in ob/ob mice was not only sustained, but increased over time, such that both fasting and ad-libitum fed BG levels were fully normalized 7 d later (FIG. 1c,d,f). Indeed, the potent anti-diabetic effect of a single icv injection of FGF1 persisted over the next 17 wk, at which point it was concluded that sustained diabetes remission had been achieved and the study was terminated (FIG. 1f). For subjects in which a single treatment provides at least 18 weeks of sustained blood glucose normalization, regular blood glucose monitoring can reveal any subsequent loss of normalization, but it is contemplated herein that the normalization provided by the single unit dose administered to the brain can continue, e.g., for 20 weeks, 24 weeks, 28 weeks, 32 weeks, or longer, e.g., one year or more, and potentially permanently. Where a single dose of any drug leads to disease remission for at least 18 weeks, it is contemplated that the drug has fundamentally altered the condition that permitted the disease state. Should the sustained effect be lost with greater time, repeat dosing is expected to return glucose normalization. Food intake and body weight were also reduced by icv mFGF1 in these mice, but the effect was transient such that the pronounced improvement of glycemia persisted for months after body weight and fat mass had returned to normal (FIG. 1g-1i). This data shows that unlike what is observed following bariatric surgery (3,4) diabetes remission induced by a single icv injection of FGF1 is fully weight loss-independent. Additionally, diabetes remission induced by icv mFGF1 in ob/ob mice was not associated with altered circulating levels of key glucoregulatory hormones (FIG. 2a-2c).

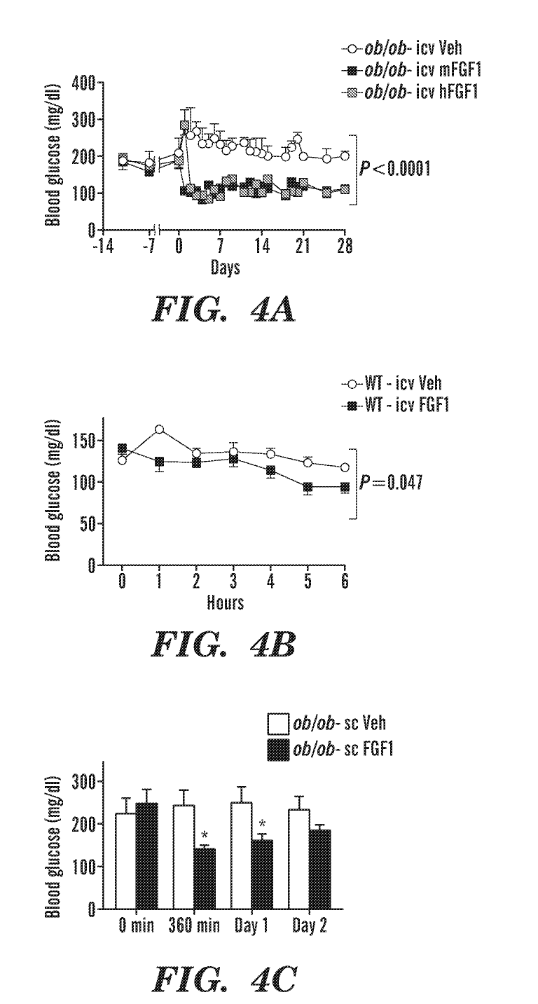

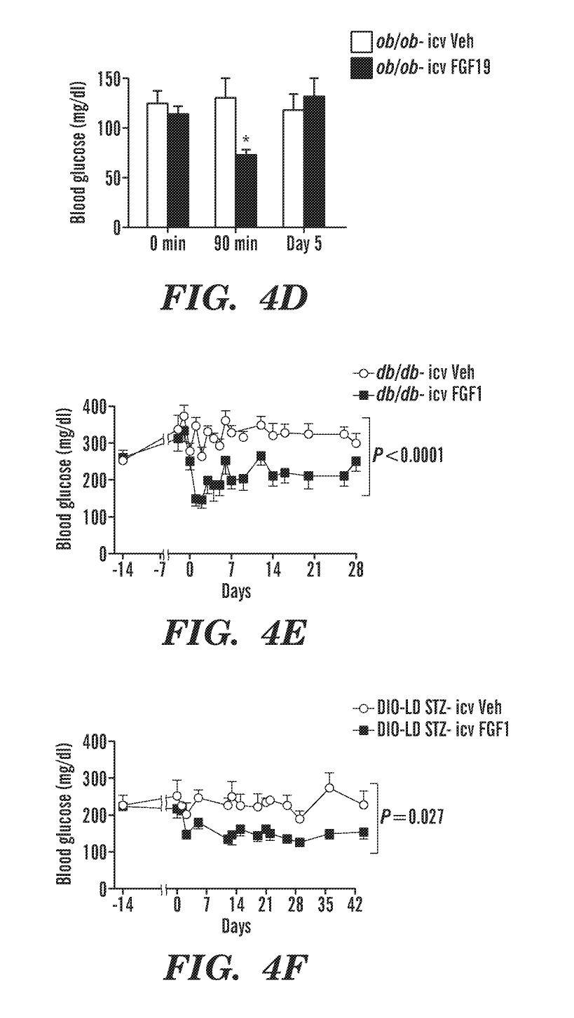

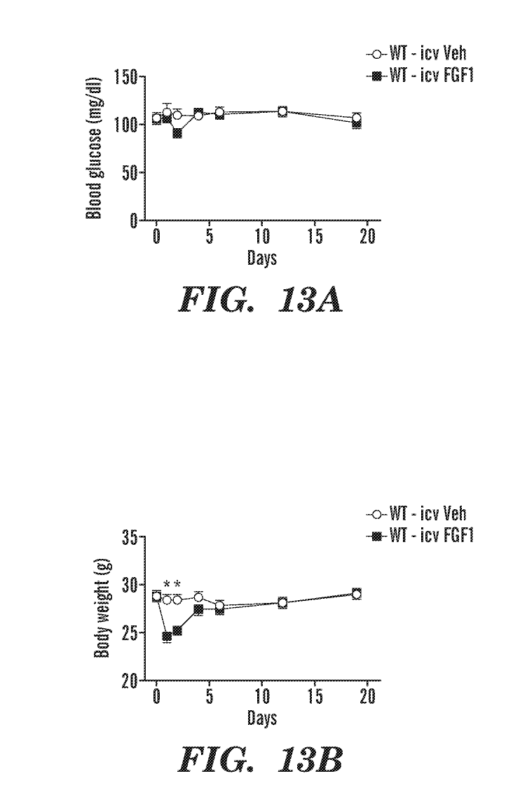

Three additional groups of diabetic ob/ob mice received a single icv injection of either vehicle (Veh), recombinant human FGF1 (hFGF1) or mFGF1. Although the onset of glucose lowering in response to hFGF1 was delayed by 24 h, sustained diabetes remission was observed following a single icv injection of either peptide (FIG. 4a). Moreover, this effect was achieved without hypoglycemia in either obese, diabetic mice (FIGS. 1f and 4a) or in lean, wild type (WT) controls (FIG. 4b) and (FIG. 13 a,b). Although this ability to elicit glucose lowering without hypoglycemia is shared by both central administration of the same dose of FGF19 (3 μg icv) and by systemic administration of a ˜10 fold higher dose of mFGF1 (0.5 mg/kg body weight sc) (FIG. 4c,d), neither intervention elicits sustained glucose lowering. Diabetes remission induced by the action of FGF1 in the brain, therefore, involves mechanisms distinct from those engaged by either systemic FGF1 or icv FGF19 when administered at doses with comparable short-term glucose-lowering efficacy.

Glycemic Response to icv FGF1 Administration Across Different Rodent Models of T2D.

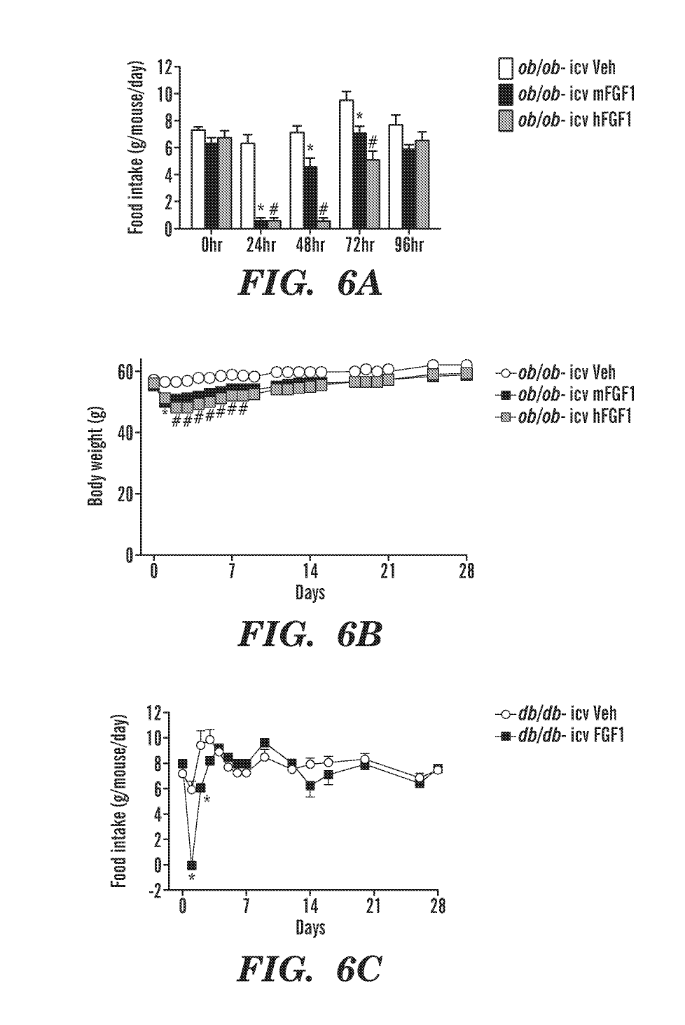

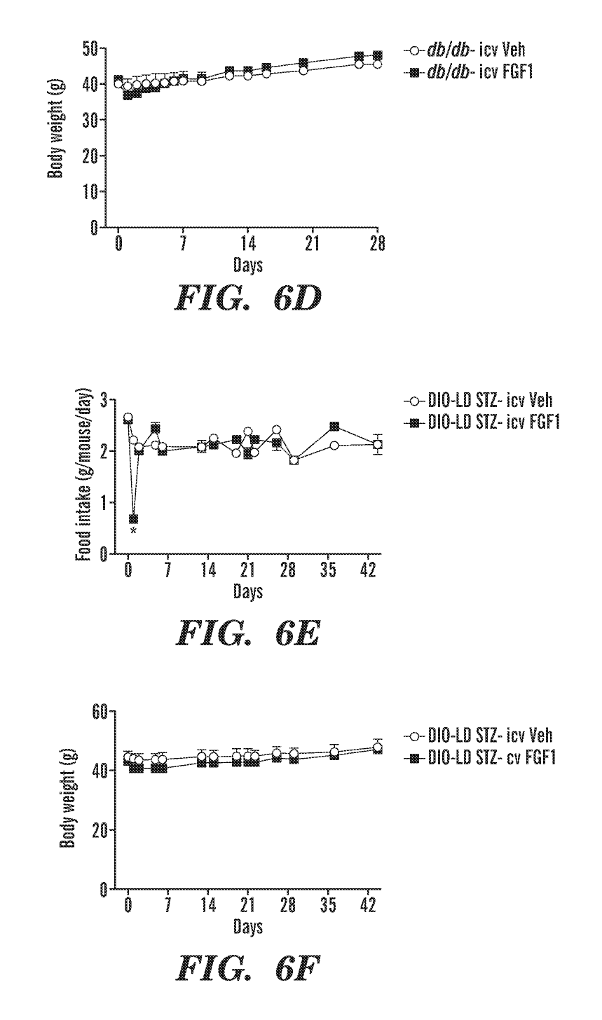

To investigate whether icv FGF1 can induce diabetes remission in other murine models of T2D, both db/db mice and WT mice in which diabetes was induced by diet-induced obesity (DIO) combined with a low dose of the β-cell toxin streptozotocin (DIO-LD STZ) were studied. As was observed in ob/ob mice (FIG. 1f, 4a), sustained diabetes remission was induced by a single icv injection of mFGF1 (3 μg) in both mouse models (FIG. 4e,f). Although reductions of both food intake and body weight were once again observed following icv FGF1 (FIG. 6a-f), these effects were transient such that pronounced glucose lowering persisted well after body weight had returned to control values (FIG. 4a,e,f).

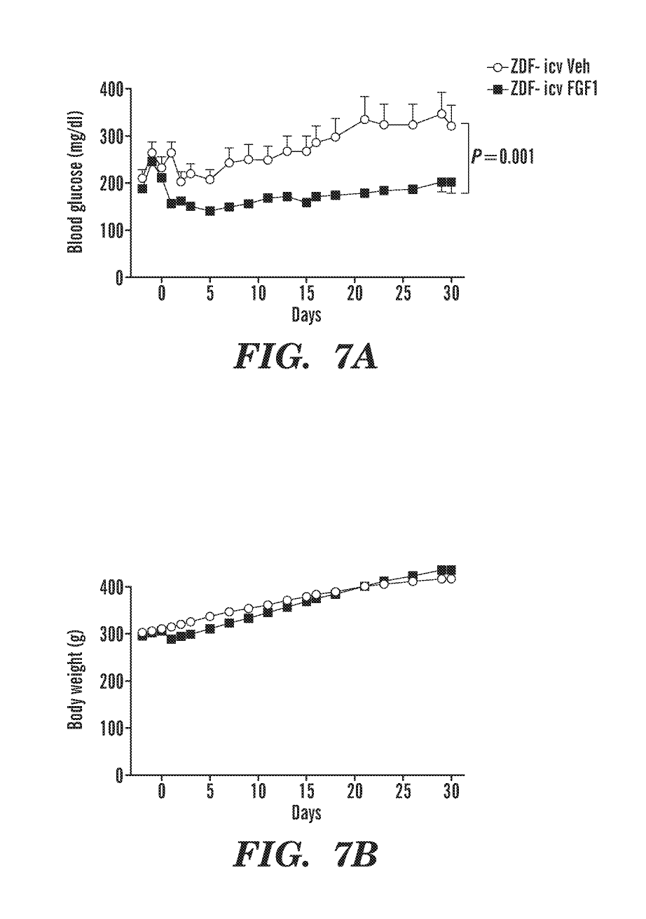

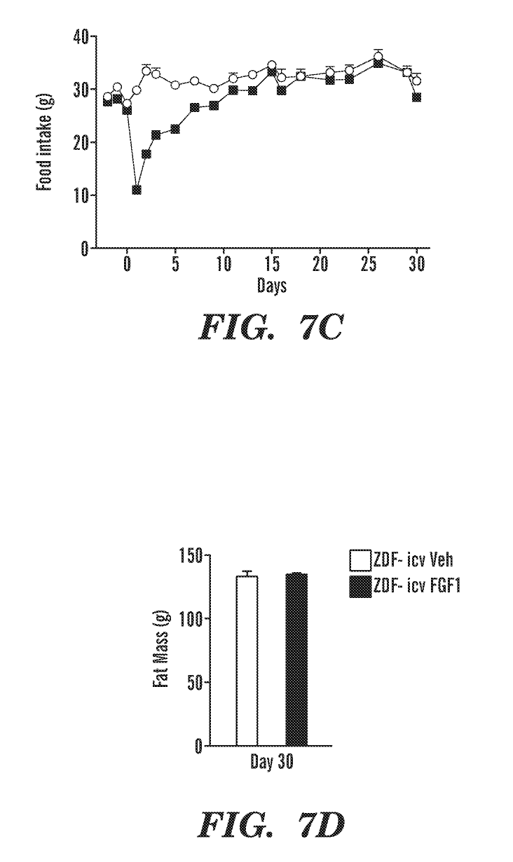

Having observed sustained diabetes remission induced by icv FGF1 in three distinct murine models of T2D, it was investigated whether this outcome is achievable in a different species. To this end, either the same dose (3 μg icv) of recombinant rat FGF1 (rFGF1) or Veh was administered to adult male Zucker Diabetic Fatty (ZDF) rats. Consistent with the findings in mice, sustained diabetes remission without hypoglycemia was induced by a single icv injection of rFGF1 in these animals (FIG. 7a). Food intake and body weight were once again reduced by icv rFGF1 (FIGS. 7b and c), but the persistence of pronounced glucose lowering well after body weight, food intake, and fat mass had returned to control values (FIG. 7b-d) demonstrates that as in mice, icv FGF1 induces weight loss-independent diabetes remission in a rat model of T2D.

Effect of icv FGF1 on Whole-Body Glucose Kinetics in Ob/Ob Mice.

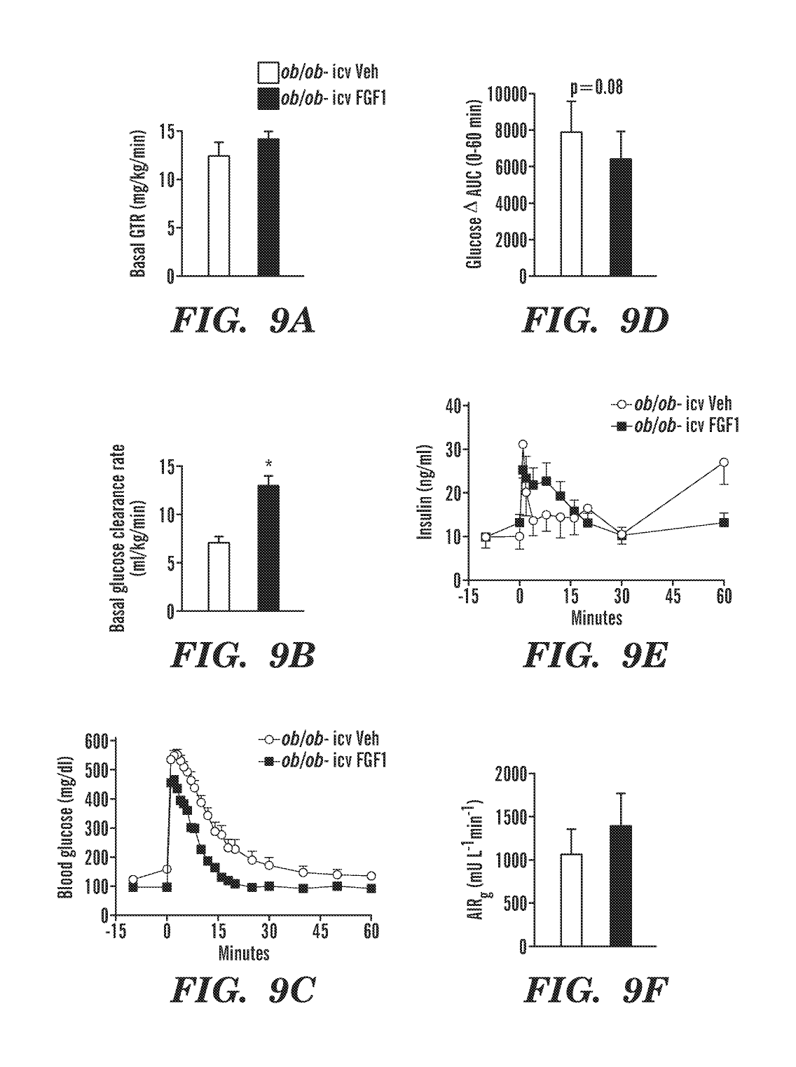

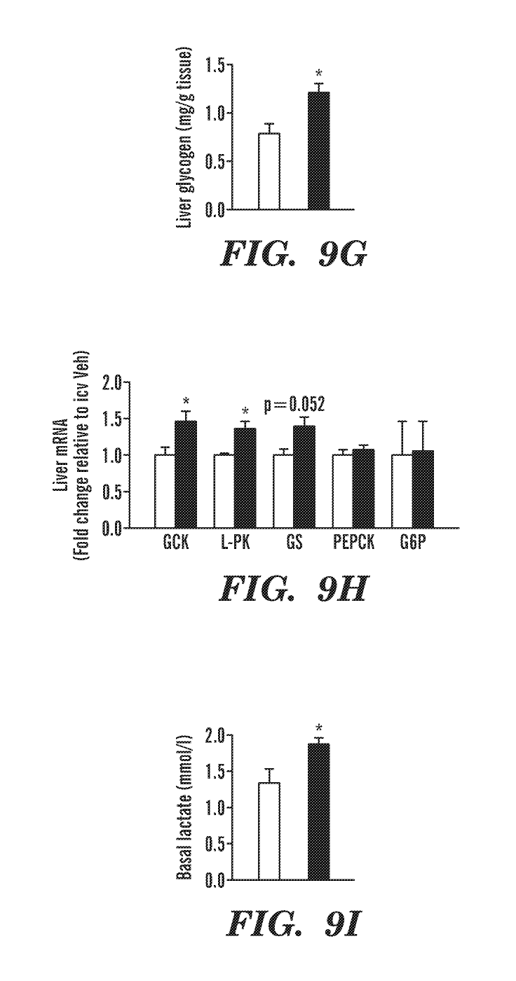

To investigate mechanisms underlying FGF1-mediated diabetes remission, basal glucose turnover in ob/ob mice receiving icv injection of either mFGF1 (3 μg) or Veh was measured. One week after icv injection, fasting BG values were reduced by ˜39% in animals receiving icv mFGF1 relative to vehicle controls (p<0.0001, t=0 min; FIG. 9c). Despite this marked reduction of BG values, the basal glucose turnover rate (GTR); which at steady-state equals the rates of both glucose production and glucose disposal) did not differ between the groups (FIG. 9a). Implied in this observation is an increase of the peripheral glucose clearance rate (a measure of the efficiency of glucose removal from the circulation), since the rate of glucose disposal increases as a function of the plasma glucose level. Indeed, the basal glucose clearance rate was increased two-fold among mice receiving icv mFGF1 compared to Veh (FIG. 9b).

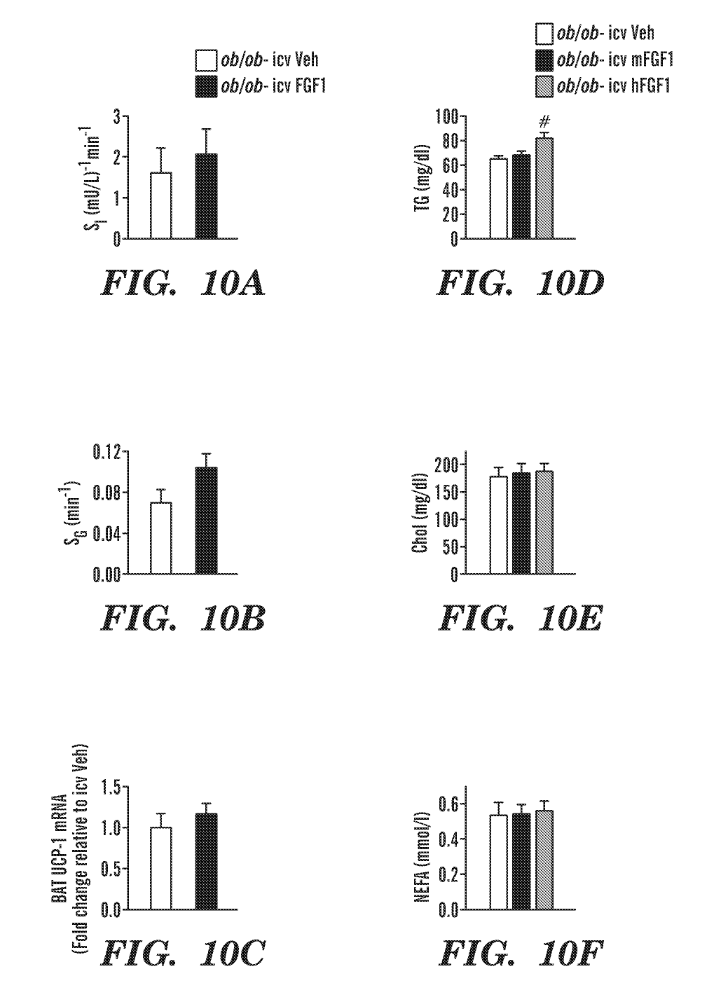

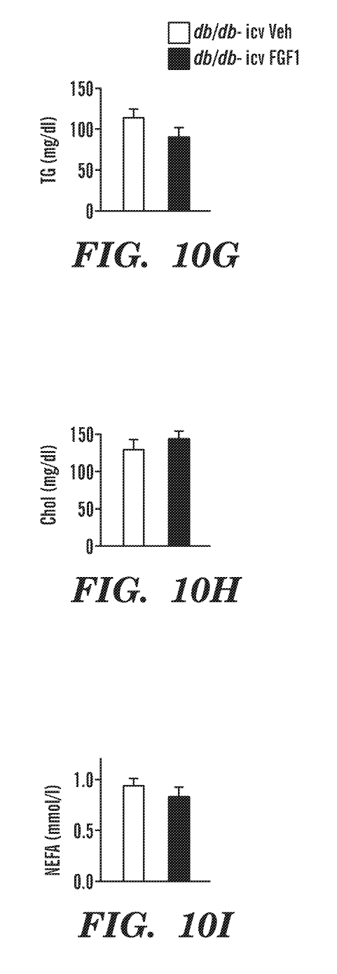

To determine whether this increase of basal glucose clearance was attributable to an increase of insulin sensitivity (measured as the insulin sensitivity index, SI), insulin secretion (measured as the acute insulin response to glucose, AIRg) or insulin-independent glucose disposal (measured as glucose effectiveness, Sg), Frequently sampled intravenous glucose tolerance test (FSIGT) followed by minimal model analysis of blood glucose and plasma insulin data (a method validated in humans, primates, dogs and rodents) was performed in the same cohort of ob/ob mice (FIG. 9c,e). Although a trend towards improved glucose tolerance was observed in mice receiving prior icv mFGF1 injection, the effect was not statistically significant after correcting for the difference in basal glucose levels (A AUC; FIG. 9d). A tendency for increased glucose-induced insulin secretion (AIRg) was also observed in the group receiving icv FGF1, but this effect again did not achieve statistical significance (FIG. 9f), nor did increases of either SI or SG (FIG. 10a,b). Sustained diabetes remission induced by the central action of FGF1, therefore, involves a novel mechanism characterized by increased peripheral glucose clearance in the basal state with no change in basal hepatic glucose production, glucose tolerance, or in any of the three determinants of glucose tolerance (insulin secretion, insulin sensitivity, and insulin-independent glucose disposal).

The liver appears to contribute substantially to the increase of basal glucose clearance induced by icv FGF1. Relative to controls receiving icv Veh, both hepatic glycogen content (FIG. 9g) and hepatic expression of genes encoding the key glucoregulatory enzymes glucokinase (GCK), liver-type pyruvate kinase (L-PK) and glycogen synthase (GS) were significantly increased in ob/ob mice 1 wk following icv mFGF1 (FIG. 9h). Combined with an increase of basal plasma lactate levels (FIG. 9i), which is consistent with increased intrahepatic glycolysis, diabetes remission induced by icv FGF1 was shown to involve increased hepatic glucose uptake (HGU) with subsequent increases of both glycogen synthesis and glycolysis.

In contrast, the expression of hepatic gluconeogenic genes phosphoenolpyruvate carboxykinase (PEPCK) and glucose-6-phosphatase (G6P) were not altered by icv mFGF1 (FIG. 9h), consistent with the absence of any effect on basal GTR (FIG. 9a). The lack of any change in uncoupling protein-1 (UCP-1) gene expression in brown adipose tissue (BAT) from ob/ob mice receiving icv mFGF1 (FIG. 10c) further suggests that activation of BAT thermogenesis does not contribute to diabetes remission induced by icv FGF1. Similarly, icv FGF1 administration was not associated with reduced plasma levels of triglycerides (TG), cholesterol (Chol) or non-esterified free fatty acids (NEFA) in either ob/ob or db/db mice (FIG. 10d-i).

Central FGF1-Mediated Glucose Lowering Requires Intact Basal Insulin Signaling.

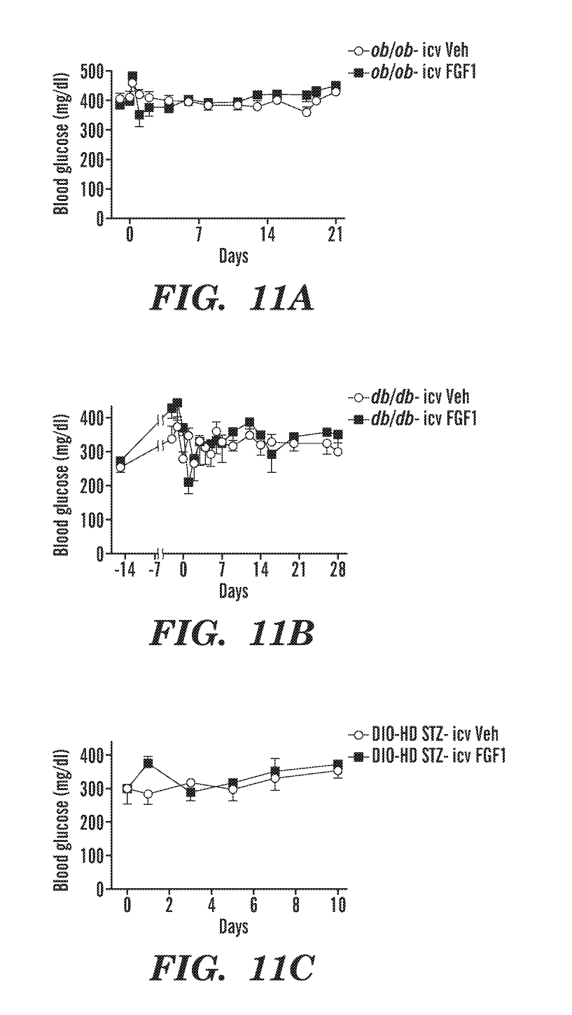

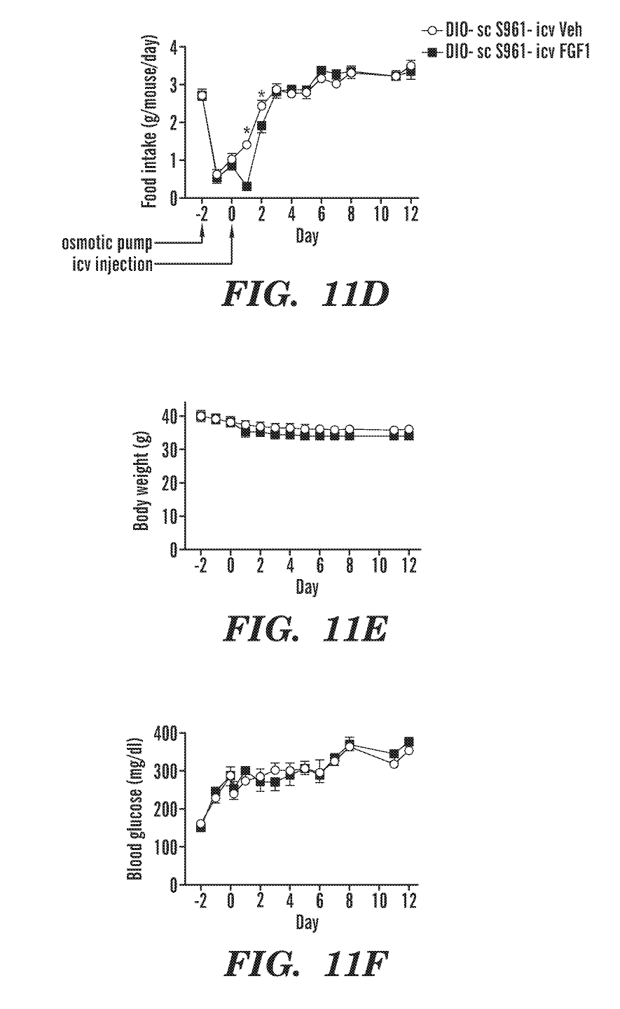

The hyperglycemia of T2D is both more stable and more moderate than in uncontrolled type 1 diabetes (T1D). This observation suggests that the pathogenesis of T2D reflects an upward re-regulation of glycemia, rather than the absence of regulation characteristic of uncontrolled T1D. This distinction provides a useful context in which to consider the findings herein, that although icv FGF1 worked well in both mouse and rat models of T2D with moderate hyperglycemia, it was ineffective in mice with severe, uncontrolled hyperglycemia (BG >300 mg/dl). This observation applies not only to db/db mice and DIO WT mice receiving a high dose of STZ, but also to ob/ob mice crossed onto the diabetogenic BTBR genetic background (FIG. 11a-c).

Without willing to be bound by theory, one potential explanation for this outcome is that glucose lowering elicited by the central action of FGF1 requires an intact insulin signal; i.e., that intact basal insulin action is permissive for diabetes remission induced by central FGF1. To test this possibility, the high-affinity insulin receptor antagonist S96134 was administered to DIO WT mice as a continuous sc infusion at a dose (29 nmol/wk) designed to achieve a level of hyperglycemia comparable to that observed in moderately diabetic ob/ob mice (on the C57B16J background; FIG. 10 that respond robustly to icv FGF1. Although icv mFGF1 transiently reduced food intake and body weight in S961-treated mice (FIG. 11d,e), it did not induce significant glucose lowering (FIG. 11f). Together, these findings show that intact insulin signaling is required for diabetes remission induced by the action of FGF1 in the brain.

### DISCUSSION

The results described herein demonstrate the brain's inherent capacity to restore normal blood glucose levels to diabetic animals in a manner that 1) is sustained for a prolonged period, and possibly indefinitely, 2) is not associated with hypoglycemia, and 3) is not secondary to changes of energy balance or fat stores. Diabetes remission induced by intracerebroventricular (icv) administration route involves a novel mechanism entailing increased glucose uptake by the liver that appears to be dependent on an intact insulin signal, since diabetes remission is blocked by systemic administration of an insulin receptor antagonist. These observations suggest that remission of T2D is feasible in humans without the need for bariatric surgery, and they extend a large literature on the brain's ability to regulate glucose homeostasis in response to input from hormonal and nutrient-related signals (4-6).

The mechanisms underlying glucose lowering elicited by systemic administration of FGF1 (21) are distinct from those activated by centrally administered FGF1. The transient nature of glucose lowering elicited by a single systemic injection of FGF1 implies that circulating FGF1 does not engage the central nervous system (CNS) mechanism responsible for sustained diabetes remission. Further, the anti-diabetic effect of systemic FGF1 reportedly requires FGFR1 signaling in adipose tissue, a mechanism that seems unlikely to explain the CNS action, since the data excludes leakage of FGF1 from brain to periphery as an explanation for diabetes remission. Whether glucose-lowering elicited by systemic administration of FGF1 (4) arises in part from transport of circulating FGF1 across the blood-brain barrier and into the brain awaits further investigation, but the effect is not prolonged, it is where the systemic effect is likely that the mechanisms differ in key ways.

The liver's enormous capacity for glucose uptake contributes substantially to glucose clearance following a meal (59) Although rising concentrations of glucose in the hepatic portal vein are considered the primary physiological mechanism driving hepatic glucose uptake (HGU), Cherrington and colleagues have demonstrated an indispensible role for signals emanating from the brain in meal-induced increases of HGU (59). These considerations raise the possibility that diabetes remission induced by icv FGF1 involves activation of neurocircuits that normally serve to enhance HGU following a meal.

Although the effect of icv FGF1 to increase basal glucose clearance occurred in the absence of significant changes of either basal insulin levels or glucose-induced insulin secretion, BG levels were reduced in FGF1-treated mice (relative to Veh-treated controls) at the time that these samples where obtained. It is therefore possible that an effect of central FGF1 to enhance insulin secretion was masked by the concurrent decrease of BG levels.

In hepatocytes, the glucose-lowering action of insulin depends on inactivation of the transcription factor FoxO1 (60). Consequently, failure to inhibit FoxO1 signaling potently reduces HGU in mice with deficient hepatic insulin signaling (61,62). While not wishing to be bound by theory, the findings described herein that systemic insulin receptor blockade negates diabetes remission induced by central administration of FGF1 is therefore compatible with a mechanism in which constitutive activation of hepatic FoxO1 explains the loss of anti-diabetic efficacy of icv FGF1 in mice with more pronounced hyperglycemia.

The findings herein point to a physiological role for hypothalamic FGF1 signaling in metabolic homeostasis. This possibility was first proposed by Oomura and colleagues (15) more than 20 years ago, based on evidence that 1) even very low doses of icv FGF1 inhibit food intake in rats, 2) FGF1 is expressed in ependymal cells lining the 3rd cerebral ventricle (adjacent to the medial hypothalamus), and 3) ependymal FGF1 appears to be released locally following a meal (15). Meal-induced release of FGF1 in this brain area may therefore convey satiety information that leads to meal termination.

Pertinent to the current work is the more recent report that FGF1-deficient mice become diabetic following the switch from standard chow to a high-fat diet (57). To explain this outcome, Evans and colleagues report that FGF1 deficiency impairs the ability of adipose tissue to remodel appropriately when confronted with nutritional excess (19). Interestingly, a recent report in zebrafish demonstrates that FGF1 deficiency similarly impairs the ability of islet beta cells to adapt to over-nutrition (63), raising the possibility that FGF1 participates in adaptive remodeling of multiple tissues in this setting. Therefore without wishing to be bound by theory, it is possible that sustained anti-diabetic action of FGF1 in the brain of animals with T2D entails remodeling of neurocircuits involved in glucose homeostasis.

Because T2D is characterized by progressive metabolic deterioration and gradually rising BG levels over time, early reports that diabetes remission can be achieved by bariatric surgery were met with skepticism—diabetes remission was simply not considered to be a feasible therapeutic outcome. In recent years, however, well-controlled studies have eliminated any doubt about the ability of bariatric procedures to achieve this goal (2,3) and the research focus has shifted to underlying mechanisms.40 Described herein for the first time is a feasible medical strategy for inducing remission of T2D without the need for surgical revision of the gastrointestinal tract. Therapeutic delivery of peptides to the brain is clearly feasible and can be enhanced for example by intranasal administration (see ref 51, 65), a possibility that has already been established for FGF1 in a mouse model (17). Chemical modification of peptides (e.g., binding the peptide to a carrier protein) can also enhance brain penetration. These strategies offer a potential path forward in efforts to translate the brain's potential to induce diabetes remission to the clinic.

In conclusion, the findings demonstrate that central administration of FGF1 unmasks the inherent capacity of the brain to induce sustained diabetes remission in mouse and rat models of T2D. This outcome is achieved without risk of hypoglycemia and without associated changes of energy balance or body fat stores. The peripheral mechanism is novel and appears to involve a centrally driven increase of glucose uptake into the liver. Strategies that target brain FGFRs are contemplated treatment options for T2D in humans.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

### REFERENCES

1. Federation, I. D. IDF Diabetes Atlas, (International Diabetes Federation (IDF), Brussels Belgium, 2015).
2. Mingrone, G., et al. Bariatric surgery versus conventional medical therapy for type 2 diabetes. The New England journal of medicine 366, 1577-1585 (2012).
3. Peterli, R., et al. Improvement in glucose metabolism after bariatric surgery: comparison of laparoscopic Roux-en-Y gastric bypass and laparoscopic sleeve gastrectomy: a prospective randomized trial. Annals of surgery 250, 234-241 (2009).
4. Schwartz, M. W., et al. Cooperation between brain and islet in glucose homeostasis and diabetes. Nature 503, 59-66 (2013).
5. Rojas, J. M. & Schwartz, M. W. Control of hepatic glucose metabolism by islet and brain. Diabetes Obes Metab 16 Suppl 1, 33-40 (2014).
6. Grayson, B. E., Seeley, R. J. & Sandoval, D. A. Wired on sugar: the role of the CNS in the regulation of glucose homeostasis. Nature reviews. Neuroscience 14, 24-37 (2013).
7. Morton, G. J., et al. FGF19 action in the brain induces insulin-independent glucose lowering. The Journal of clinical investigation 123, 4799-4808 (2013).
8. Marcelin, G., et al. Central action of FGF19 reduces hypothalamic AGRP/NPY neuron activity and improves glucose metabolism. Molecular metabolism 3, 19-28 (2014).
9. Ryan, K. K., et al. Fibroblast growth factor-19 action in the brain reduces food intake and body weight and improves glucose tolerance in male rats. Endocrinology 154, 9-15 (2013).
10. Fu, L., et al. Fibroblast growth factor 19 increases metabolic rate and reverses dietary and leptin-deficient diabetes. Endocrinology 145, 2594-2603 (2004).
11. Sarruf, D. A., et al. Fibroblast growth factor 21 action in the brain increases energy expenditure and insulin sensitivity in obese rats. Diabetes 59, 1817-1824 (2010).
12. Owen, B. M., et al. FGF21 acts centrally to induce sympathetic nerve activity, energy expenditure, and weight loss. Cell metabolism 20, 670-677 (2014).
13. Raju, R., et al. A Network Map of FGF-1/FGFR Signaling System. Journal of signal transduction 2014, 962962 (2014).
14. Ito, J., et al. Astrocytes produce and secrete FGF-1, which promotes the production of apoE-HDL in a manner of autocrine action. Journal of lipid research 46, 679-686 (2005).
15. Oomura, Y., et al. A new brain glucosensor and its physiological significance. The American journal of clinical nutrition 55, 278S-282S (1992).
16. Suzuki, S., et al. Feeding suppression by fibroblast growth factor-1 is accompanied by selective induction of heat shock protein 27 in hypothalamic astrocytes. The European journal of neuroscience 13, 2299-2308 (2001).
17. Lou, G., et al. Intranasal administration of TAT-haFGF((1)(4)(-)(1)(5)(4)) attenuates disease progression in a mouse model of Alzheimer's disease. Neuroscience 223, 225-237 (2012).
18. Cheng, X., et al. Acidic fibroblast growth factor delivered intranasally induces neurogenesis and angiogenesis in rats after ischemic stroke. Neurological research 33, 675-680 (2011).
19. Jonker, J. W., et al. A PPARgamma-FGF1 axis is required for adaptive adipose remodelling and metabolic homeostasis. Nature 485, 391-394 (2012).
20. Ornitz, D. M. & Itoh, N. The Fibroblast Growth Factor signaling pathway. Wiley interdisciplinary reviews. Developmental biology 4, 215-266 (2015).
21. Suh, J. M., et al. Endocrinization of FGF1 produces a neomorphic and potent insulin sensitizer. Nature 513, 436-439 (2014).
22. Xu, J., et al. Acute glucose-lowering and insulin-sensitizing action of FGF21 in insulin-resistant mouse models—association with liver and adipose tissue effects. Am J Physiol Endocrinol Metab 297, E1105-1114 (2009).
23. Prevention, C.f.D.C.a. National Diabetes Statistics Report: Estimates of Diabetes and Its Burden in the United States, 2014. (U.S. Department of Health and Human Services, Atlanta, Ga., 2014).
24. Aileen J F King. The use of animal models in diabetes research. Br J Pharmacol. 2012 June; 166(3): 877-894 (2012).
25. Papahadjopoulos Demetrios P., et al. Method of encapsulating biologically active materials in lipid vesicles U.S. Pat. No. 4,235,871 A (1980)
26. Rosenberg J, et. al. Prostaglandin e1 formulation CA 2113443 A1 (1993).
27. Jessee, J et. al., Genetic immunization with cationic lipids US 20070141677 A1 (2007).
28. Paul Behr Jean, et. al Lipopolyamines, their preparation and their use U.S. Pat. No. 5,171,678 A (1992).
29. Gruell, H., et. al. Carriers for the local release of hydrophilic prodrugs EP 2667848 A1.
30. Niven, R W et. al. Methods for preparing lipids/polynucleotide transfection complexes WO 1999015206 A1 (1999).
31. Buzea, C. et. al. Nanomaterials and nanoparticles: sources and toxicity., Biointerphases. December; 2(4):MR17-71 (2007).
32. Mudshinge, S R. et. al. Nanoparticles: Emerging carriers for drug delivery. Saudi Pharm J. July; 19(3):129-41 (2011).
33. Shah, L. Nanotechnology for CNS delivery of bio-therapeutic agents. Drug Deliv Transl Res. August 1; 3(4):336-51 (2013).
34. Kreuter, J. Nanoparticles made of protein with coupled apolipoprotein e for penetration of the blood-brain barrier and methods for the production thereof US 2004/0131692 A1
35. Fukuta, M. et al. Peptide conjugate. US005442043A (1995).
36. Ornitz D M1., FGFs, heparan sulfate and FGFRs: complex interactions essential for development. Bioessays. February; 22(2):108-12 (2000).
37. Jonker, J W. Methods for treating metabolic disorders using FGF U.S. Pat. No. 8,906,854 B2 (2014)
38. Jae Myoung Suh, Mutated fibroblast growth factor (fgf) 1 and methods of use US 20150111821 A1 (2015).
39. Mohammadi, M., Fibroblast growth factor 1 protein fragments and methods of use US 20140155316 A1, (2014).
40. Misra, A. Drug delivery to the central nervous system: a review. J Pharm Pharm Sci. May-August; 6(2):252-73 (2003).
41. Tice, TR. METHOD FOR STIMULATING NERVE FIBER GROWTH, US005360610A (1994).
42. Barchet, T M. Challenges and opportunities in CNS delivery of therapeutics for neurodegenerative diseases. Expert Opin Drug Deliv. March; 6(3):211-25 (2009).
43. Ganta et al., A review of multifunctional nanoemulsion systems to overcome oral and CNS drug delivery barriers Mol Membr Biol. October; 27(7):260-73 (2010).
44. Micklus, M Targeting of liposomes to the blood-brain barrier. US 20020025313 A1, (2002)
45. Zara et al., J Drug Target 2002 June; 10(4):327-35.
46. Di Stefano et al., Expert Opin. Drug Deliv. 2011 May; 8(5):581-603.
47. Pardridge William M. Chimeric peptides for neuropeptide delivery through the blood-brain barrier, U.S. Pat. No. 4,801,575A, (1989).
48. Wu, Dafang et. al. Transport of basic fibroblast growth factor across the blood brain barrier US 20040102369 A1 (2004).
49. Stalmans S. Cell-Penetrating Peptides Selectively Cross the Blood-Brain Barrier In Vivo. PLoS One. 2015 Oct. 14; 10(10):e0139652.
50. Ren Zhong Xiao et. al. Recent advances in PEG-PLA block copolymer nanoparticles Int. J. Nanomedicine 5: 1057-1065 (2010).
51. Franciscus W. H. M. Merkus, Pharmaceutical compositions for intranasal administration of apomorphine U.S. Pat. No. 5,756,483 A, (1998).
52. Kravtzoff et al., Preclinical toxicology of Biovector™ nanoparticles: part II, local tolerance, genetic toxicology and pharmacokinetics Toxicol. Lett. 95: 117 (1998).
53. William, Frey H. II Method for administering fibroblast growth factor to the brain, U.S. Pat. No. 6,342,478 B1, (1999).
54. Perez A P. Increased brain radioactivity by intranasal P-labeled siRNA dendriplexes within in situ-forming mucoadhesive gels. Int J Nanomedicine. 2012; 7:1373-85. doi: 10.2147/IJN.S28261.
55. Nikol et al., Mol Ther. 16: 972-978 (2008).
56. Ayala, J. E., Bracy, D. P., McGuinness, O. P. & Wasserman, D. H. Considerations in the design of hyperinsulinemic-euglycemic clamps in the conscious mouse. Diabetes 55, 390-397 (2006).
57. Best, J. D., Taborsky, G. J., Jr., Halter, J. B. & Porte, D., Jr. Glucose disposal is not proportional to plasma glucose level in man. Diabetes 30, 847-850 (1981).
58. Alonso, L. C., et al. Simultaneous measurement of insulin sensitivity, insulin secretion, and the disposition index in conscious unhandled mice. Obesity (Silver Spring) 20, 1403-1412 (2012).
59. Moore, M. C., Coate, K. C., Winnick, J. J., An, Z. & Cherrington, A. D. Regulation of hepatic glucose uptake and storage in vivo. Adv Nutr 3, 286-294 (2012).
60. Lu, M., et al. Insulin regulates liver metabolism in vivo in the absence of hepatic Akt and Foxol. Nature medicine 18, 388-395 (2012).
61. I, O. S., et al. FoxO1 integrates direct and indirect effects of insulin on hepatic glucose production and glucose utilization. Nature communications 6, 7079 (2015).
62. Titchenell, P. M., Chu, Q., Monks, B. R. & Birnbaum, M. J. Hepatic insulin signalling is dispensable for suppression of glucose output by insulin in vivo. Nature communications 6, 7078 (2015).
63. Li, M., Page-McCaw, P. & Chen, W. FGF1 Mediates Overnutrition-Induced Compensatory beta-Cell Differentiation. Diabetes (2015).
64. Knop, F. K. & Taylor, R. Mechanism of metabolic advantages after bariatric surgery: it's all gastrointestinal factors versus it's all food restriction. Diabetes care 36 Suppl 2, S287-291 (2013).
65. Mittal, D., et al. Insights into direct nose to brain delivery: current status and future perspective. Drug delivery 21, 75-86 (2014).
66. Born, J. et. al. Sniffing neuropeptides: a transnasal approach to the human brain Nature Neuroscience 5, 514-516 (2002).
67. Dittert, L. et. al. Method for the intranasal administration of 1-dopa prodrugs WO 1997016181 A1, (1997)

1. A pharmaceutical composition comprising a unit dose of Fibroblast Growth Factor 1 (FGF1) polypeptide preparation comprising a pharmaceutically acceptable carrier and formulated for administration to the brain

2. The composition of claim 1, wherein the composition is formulated for administration via an intracerebroventricular, intranasal, intracranial, intracelial, intracerebellar, or intrathecal administration route.

3. The composition of claim 2, wherein the composition is formulated for administration via an intranasal route and further comprises a ganglioside and/or a phosphotidylserine.

4. The composition of claim 2, wherein the composition is formulated for administration via an intranasal route and further comprises saccharides selected from the group of cyclodextrins, disaccharides, polysaccharides, and combinations thereof.

5. (canceled)

6. The pharmaceutical composition of claim 1, wherein the FGF1 polypeptide is a human FGF1 polypeptide.

7-10. (canceled)

11. The pharmaceutical composition of claim 1, wherein the composition is contained in a delivery device selected from the group consisting of a syringe, a blunt tip syringe, a catheter, an inhaler, a nebulizer, a nasal spray pump, a nasal irrigation pump or nasal lavage pump, and an implantable pump.

12. The pharmaceutical composition of claim 1, wherein the FGF1 polypeptide is formulated with a lipophilic molecular group.

13. The pharmaceutical composition of claim 1, wherein the FGF1 polypeptide is encapsulated in a liposome or a nanoparticle.

14. The pharmaceutical composition of claim 1 wherein the FGF1 polypeptide is fused to a carrier polypeptide.

15. The pharmaceutical composition of claim 1 wherein unit dose of Fibroblast Growth Factor 1 (FGF1) polypeptide is less than 50% of the unit dose required to treat diabetes via systemic administration.

16. The pharmaceutical composition of claim 1 wherein the unit dose comprises less than about 30 μg of the FGF1 polypeptide.

17-20. (canceled)

21. A method of treating a metabolic disorder in a subject, the method comprising administering a unit dose of a pharmaceutical composition comprising a Fibroblast Growth Factor 1 (FGF1) polypeptide preparation of claim 1 to the brain of a subject having a metabolic disorder, wherein the metabolic disorder is treated.

22. (canceled)

23. The method of claim 21, wherein the metabolic disorder is a disorder characterized by or involving abnormally elevated blood glucose levels.

24. (canceled)

25. The method of claim 21, further comprising the step, prior to the administering step, of diagnosing the patient as having a metabolic disorder.

26. The method of claim 21, wherein prior to administration of the pharmaceutical composition the subject has a blood glucose level above the normal range, and wherein administration of the composition lowers blood glucose level to within the normal range.

27-30. (canceled)

31. The method of claim 21, wherein a single unit dose of the administered pharmaceutical composition normalizes blood glucose level in the subject for at least one week.

32. (canceled)

33. (canceled)

34. The method of claim 21, further comprising administering one or more agents selected from the group consisting of an anti-inflammatory agent, an anti-fibrotic agent, an anti-hypertensive agent, an anti-diabetic agent, a triglyceride lowering agent, and a cholesterol lowering agent to the subject.

35-42. (canceled)

43. The method of claim 21, wherein the blood glucose levels are normalized in 1 week or less after a single administration of the pharmaceutical composition.

44-52. (canceled)

53. A method to treat high blood glucose levels in a subject in need thereof, comprising administering a therapeutically effective amount of an FGFR binding protein to the brain of the subject to normalize the blood glucose levels to normal range, wherein the FGFR is selected from the group, FGFR1, FGFR2, FGFR3, FGFR4 or a combination thereof.

54. The method of claim 53 wherein the FGFR binding protein is a FGF1 polypeptide.

55-74. (canceled)


SEQUENCE LISTING (108 sequences)

SEQ ID NO: 1 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 2 - protein, 131 aa, Homo sapiens
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 3 - protein, 127 aa, Homo sapiens
LYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 4 - protein, 155 aa, Mus musculus
MAEGEITTFAALTERFNLPLGNYKKPKLLYCSNGGHFLRILPDGTVDGTRDRSDQHIQLQLSAESAGEVYIKGTETGQYLAMDTEGLLYGSQTPNEECLFLERLEENHYNTYTSKKHAEKNWFVGLKKNGSCKRGPRTHYGQKAILFLPLPVSSD

SEQ ID NO: 5 - protein, 155 aa, Rattus norvegicus; identical to SEQ ID NO: 4

SEQ ID NO: 6 - protein, 155 aa, Bos taurus
MAEGETTTFTALTEKFNLPLGNYKKPKLLYCSNGGYFLRILPDGTVDGTKDRSDQHIQLQLCAESIGEVYIKSTETGQFLAMDTDGLLYGSQTPNEECLFLERLEENHYNTYISKKHAEKHWFVGLKKNGRSKLGPRTHFGQKAILFLPLPVSSD

SEQ ID NO: 7 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 8 - protein, 155 aa, Homo sapiens [sequence omitted]

SEQ ID NO: 9 - protein, 154 aa, Mus musculus
MAASGITSLPALPEDGGAAFPPGHFKDPKRLYCKNGGFFLRIHPDGRVDGVREKSDPHVKLQLQAEERGVVSIKGVCANRYLAMKEDGRLLASKCVTEECFFFERLESNNYNTYRSRKYSSWYVALKRTGQYKLGSKTGPGQKAILFLPMSAKS

SEQ ID NO: 10 - protein, 154 aa, Rattus norvegicus
MAAGSITSLPALPEDGGGAFPPGHFKDPKRLYCKNGGFFLRIHPDGRVDGVREKSDPHVKLQLQAEERGVVSIKGVCANRYLAMKEDGRLLASKCVTEECFFFERLESNNYNTYRSRKYSSWYVALKRTGQYKLGSKTGPGQKAILFLPMSAKS

SEQ ID NO: 11 - protein, 155 aa, Bos taurus
MAAGSITTLPSLPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKS

SEQ ID NO: 12 - protein, 239 aa, Homo sapiens
MGLIWLLLLSLLEPGWPAAGPGARLRRDAGGRGGVYEHLGGAPRRRKLYCATKYHLQLHPSGRVNGSLENSAYSILEITAVEVGIVAIRGLFSGRYLAMNKRGRLYASEHYSAECEFVERIHELGYNTYASRLYRTVSSTPGARRQPSAERLWYVSVNGKGRPRRGFKTRRTQKSSLFLPRVLDHRDHEMVRQLQSGLPRPPGKGVQPRRRRQKQSPDNLEPSHVQASRLGSQLEASAH

SEQ ID NO: 13 - protein, 245 aa, Mus musculus
MGLIWLLLLSLLEPSWPTTGPGTRLRRDAGGRGGVYEHLGGAPRRRKLYCATKYHLQLHPSGRVNGSLENSAYSILEITAVEVGVVAIKGLFSGRYLAMNKRGRLYASDHYNAECEFVERIHELGYNTYASRLYRTGSSGPGAQRQPGAQRPWYVSVNGKGRPRRGFKTRRTQKSSLFLPRVLGHKDHEMVRLLQSSQPRAPGEGSQPRQRRQKKQSPSDHGKMETLSTRATPSTQLHTGGLAVA

SEQ ID NO: 14 - protein, 245 aa, Rattus norvegicus
MGLIWLLLLSLLEPGWPATGPGTRLRRDAGGRGGVYEHLGGAPRRRKLYCATKYHLQLHPSGRVNGSLENSAYSILEITAVEVGVVAIKGLFSGRYLAMNKRGRLYASEHYNAECEFVERIHELGYNTYASRLYRTGPSGPGARRQPGAQRPWYVSVNGKGRPRRGFKTRRTQKSSLFLPRVLGHKDHEMVRLLQSGQPQAPGEGSQPRQRRQKKQSPGDHGKMEHLPTKATTSAQLDTGGLAMA

SEQ ID NO: 15 - protein, 236 aa, Bos taurus
MDLIWLLLLSLLEPGWPAAGPVARPRRDAGGRGGVYEHLGGAPRRRKLYCATKYHLQLHPSGRVNGSLENSAYSILEITAVEVGVVAIKGLFSGRYLAMNKRGRLYASESYNAECEFVERIHELGYNTYASRLYRTAPSGRGARRQPSAERLWYVSVNGKGRPRRGFKTRRTQKSSLFLPRVLDRKDHEMVRLLLGTAGLRGGQARPPPPGRAASMRQRRRRQQRRPRDRDRGGRA

SEQ ID NO: 16 - protein, 206 aa, Homo sapiens
MSGPGTAAVALLPAVLLALLAPWAGRGGAAAPTAPNGTLEAELERRWESLVALSLARLPVAAQPKEAAVQSGAGDYLLGIKRLRRLYCNVGIGFHLQALPDGRIGGAHADTRDSLLELSPVERGVVSIFGVASRFFVAMSSKGKLYGSPFFTDECTFKEILLPNNYNAYESYKYPGMFIALSKNGKTKKGNRVSPTMKVTHFLPRL

SEQ ID NO: 17 - protein, 202 aa, Mus musculus
MAKRGPTTGTLLPRVLLALVVALADRGTAAPNGTRHAELGHGWDGLVARSLARLPVAAQPPQAAVRSGAGDYLLGLKRLRRLYCNVGIGFHLQVLPDGRIGGVHADTRDSLLELSPVQRGVVSIFGVASRFFVAMSSRGKLFGVPFFTDECKFKEILLPNNYNAYESYAYPGMFMALSKNGRTKKGNRVSPTMKVTHFLPRL

SEQ ID NO: 18 - protein, 195 aa, Rattus norvegicus
MAKRGPTTGTLLPGVLLALVVALADRGTAAPNGTRHAELGHGWDGLVARSLARLPVAAQPPHAAVRSGAGDYLLGLKRLRRLYCNVGIGFHLQVLPDGRIGGVPRGHEGQQRGVVSIFGVASRFFVAMSSRGKLFGVPFFTDECKFKEILLPNNYNAYESYAYPGMFMALSKNGRTKKGNRVSPTMKVTHFLPRL

SEQ ID NO: 19 - protein, 206 aa, Bos taurus
MAGPGAAAAALLPAVLLAVLAPWAGRGGAAAPTAPNGTLEAELERRWESLVARSLARLPVAAQPKEAAVQSGAGDYLLGIKRLRRLYCNVGIGFHLQVLPDGRIGGVHADTSDSLLELSPVERGVVSIFGVASRFFVAMSSRGRLYGSPFFTDECRFREILLPNNYNAYECDRHPGMFIALSKNGKAKKGNRVSPTMKVTHFLPRL

SEQ ID NO: 20 - protein, 268 aa, Homo sapiens
MSLSFLLLLFFSHLILSAWAHGEKRLAPKGQPGPAATDRNPRGSSSRQSSSSAMSSSSASSSPAASLGSQGSGLEQSSFQWSPSGRRTGSLYCRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKKPPSPIKPKIPLSAPRKNTNSVKYRLKFRFG

SEQ ID NO: 21 - protein, 264 aa, Mus musculus
MSLSLLFLIFCSHLIHSAWAHGEKRLTPEGQPAPPRNPGDSSGSRGRSSATFSSSSASSPVAASPGSQGSGSEHSSFQWSPSGRRTGSLYCRVGIGFHLQIYPDGKVNGSHEASVLSILEIFAVSQGIVGIRGVFSNKFLAMSKKGKLHASAKFTDDCKFRERFQENSYNTYASAIHRTEKTGREWYVALNKRGKAKRGCSPRVKPQHVSTHFLPRFKQSEQPELSFTVTVPEKKKPPVKPKVPLSQPRRSPSPVKYRLKFRFG

SEQ ID NO: 22 - protein, 266 aa, Rattus norvegicus
MSLSLLFLIFCSHLILSAPAQGEKRLTPEGQPAPPRNPGDSSGSRGRSSATFASSSASSPVAASPGSQGSGSEHSSFQWSPSGRRTGSLYCRVGIGFHLQIYPDGKVNGSHEASVLSILEIFAVSQGIVGIRGVFSNKFLAMSKKGKLHASAKFTDDCKFRERFQENSYNTYASAIHRTEKTGREWYVALNKRGKAKRGCSPRVKPQHVSTHFLPRFKQSEQPELSFTVTVPEKKKPPSPVKPKVPLSPPRRSPSPVKYRLKFRFG

SEQ ID NO: 23 - protein, 270 aa, Bos taurus
MSLSFLLLLFLSHLILSAWAQGEKRLAPKGQPGPAATERNPGGASSRRSSSSTATSSSSPASSSSAASRGGPGSSLEQSSFQWSPSGRRTGSLYCRVGIGFHLQIYPDGKVNGSHEANMLSILEIFAVSQGIVGIRGVFSNKFLAMSKKGKLHASAKFTDDCKFRERFQENSYNTYASAIHRTEKTGREWYVALNKRGKAKRGCSPRVKPQHVSTHFLPRFKQLEQPELSFTVTVPEKKKPPNPVKPKVPLSAPRRSPNTVKYRLKFRFG

SEQ ID NO: 24 - protein, 208 aa, Homo sapiens
MALGQKLFITMSRGAGRLQGTLWALVFLGILVGMVVPSPAGTRANNTLLDSRGWGTLLSRSRAGLAGEIAGVNWESGYLVGIKRQRRLYCNVGIGFHLQVLPDGRISGTHEENPYSLLEISTVERGVVSLFGVRSALFVAMNSKGRLYATPSFQEECKFRETLLPNNYNAYESDLYQGTYIALSKYGRVKRGSKVSPIMTVTHFLPRI

SEQ ID NO: 25 - protein, 208 aa, Mus musculus
MALGQRLFITMSRGAGRVQGTLQALVFLGVLVGMVVPSPAGARANGTLLDSRGWGTLLSRSRAGLAGEISGVNWESGYLVGIKRQRRLYCNVGIGFHLQVPPDGRISGTHEENPYSLLEISTVERGVVSLFGVKSALFIAMNSKGRLYTTPSFHDECKFRETLLPNNYNAYESDLYRGTYIALSKYGRVKRGSKVSPIMTVTHFLPRI

SEQ ID NO: 26 - protein, 208 aa, Rattus norvegicus
MALGQRLFITMSRGAGRVQGTLQALVFLGVLVGMVVPSPAGARANGTLLDSRGWGTLLSRSRAGLAGEISGVNWESGYLVGIKRQRRLYCNVGIGFHLQVPPDGRISGTHEENPYSLLEISTVERGVVSLFGVKSALFIAMNSKGRLYTTPSFQDECKFRETLLPNNYNAYESDLYRGTYIALSKYGRVKRGSKVSPIMTVTHFLPRI

SEQ ID NO: 27 - protein, 208 aa, Bos taurus
MARGQTPLITMSRGAGRPQGTLRALVFLGVLVGMVVPSPAGTRANGTLLASRGWGTLLSRSRAGLAGEIAGVNWESGYLVGIKRQRRLYCNVGIGFHLQVPPDGRISGTHEENPYSLLEISTVERGVVSLFGVKSALFVAMNSKGKLYATPSFQEECKFRETLLPNNYNAYESDLYRGAYIALSKYGRVKRGSKVSPTMTVTHFLPRI

SEQ ID NO: 28 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 29 - protein, 194 aa, Mus musculus
MRKWILTRILPTLLYRSCFHLVCLVGTISLACNDMSPEQTATSVNCSSPERHTRSYDYMEGGDIRVRRLFCRTQWYLRIDKRGKVKGTQEMKNSYNIMEIRTVAVGIVAIKGVESEYYLAMNKEGKLYAKKECNEDCNFKELILENHYNTYASAKWTHSGGEMFVALNQKGIPVKGKKTKKEQKTAHFLPMAIT

SEQ ID NO: 30 - protein, 194 aa, Rattus norvegicus
MRKWILTRILPTPLYRSCFHLVCLVGTISLACNDMSPEQTATSVNCSSPERHTRSYDYMEGGDIRVRRLFCRTQWYLRIDKRGKVKGTQEMRNSYNIMEIRTVAVGIVAIKGVESEYYLAMNKEGKLYAKKECNEDCNFKELILENHYNTYASAKWTHSGGEMFVALNQKGLPVKGKKTKKEQKTAHFLPMAIT

SEQ ID NO: 31 - protein, 194 aa, Bos taurus
MRKWILTWILPSLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEYYLAMNKEGKLYAKKECNEDCNFKELILENHYNTYASAKWTHSGGEMFVALNQKGVPVRGKKTKKEQKTAHFLPMAIT

SEQ ID NO: 32 - protein, 233 aa, Homo sapiens
MGSPRSALSCLLLHLLVLCLQAQEGPGRGPALGRELASLFRAGREPQGVSQQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 33 - protein, 244 aa, Mus musculus
MGSPRSALSCLLLHLLVLCLQAQEGPGGGPALGREPTSLLRAGREPQGVSQQVTVQSSPNFTQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 34 - protein, 204 aa, Rattus norvegicus
MGSPRSALSCLLLHLLVLCLQAQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 35 - protein, 244 aa, Bos taurus
MGSPRSALSCLLLHLLVLCLQAQEGPGGGPALGRELASLFRAGRESQGVSQQVTVQSSPNFTQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 36 - protein, 208 aa, Homo sapiens
MAPLGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 37 - protein, 208 aa, Mus musculus
MAPLGEVGSYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRCYVALNKDGTPREGTRTKRHQKFTHFLPRPVDPDKVPELYKDILSQS

SEQ ID NO: 38 - protein, 208 aa, Rattus norvegicus
MAPLGEVGSYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 39 - protein, 208 aa, Bos taurus
MAPLGEVGNYFGVQDAVPFGNGPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRFYVALNKDGTPREGTRTKRHQKFTHFLPRPVDPDKVPELYKDILSQS

SEQ ID NO: 40 - protein, 208 aa, Homo sapiens
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 41 - protein, 209 aa, Mus musculus
MWKWILTHCASAFPHLPGCCCCFLLLFLVSSFPVTCQALGQDMVSQEATNCSSSSSSFSSPSSAGRHVRSYNHLQGDVRWRRLFSFTKYFLTIEKNGKVSGTKNEDCPYSVLEITSVEIGVVAVKAINSNYYLAMNKKGKLYGSKEFNNDCKLKERIEENGYNTYASFNWQHNGRQMYVALNGKGAPRRGQKTRRKNTSAHFLPMTIQT

SEQ ID NO: 42 - protein, 215 aa, Rattus norvegicus
MWKWILTHCASAFPHLPGCCCCFLLLFLVSSVPVTCQALGQDMVSPEATNSSSSSSSSSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 43 - protein, 213 aa, Bos taurus
MWKWILTHCASAFPHLSGCCCCFLLLFLVSSVPVTCQALDQDMVSPGATNSSSSSSSSSSSSVSLPSSAGRHVRSYNHLQGDVRWRKLFSFTKYFLKIENGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 44 - protein, 225 aa, Homo sapiens
MAALASSLIRQKREVREPGGSRPVSAQRRVCPRGTKSLCQKQLLILLSKVRLCGGRPARPDRGPEPQLKGIVTKLFCRQGFYLQANPDGSIQGTPEDTSSFTHFNLIPVGLRVVTIQSAKLGHYMAMNAEGLLYSSPHFTAECRFKECVFENYYVLYASALYRQRRSGRAWYLGLDKEGQVMKGNRVKKTKAAAHFLPKLLEVAMYQEPSLHSVPEASPSSPPAP

SEQ ID NO: 45 - protein, 197 aa, Mus musculus
MAALASSLIRQKREVREPGGSRPVSAQRRVCPRGTKSLCQKQLLILLSKVRLCGGRPTRQDRGPEPQLKGIVTKLFCRQGFYLQANPDGSIQGTPEDTSSFTHFNLIPVGLRVVTIQSAKLGHYMAMNAEGLLYSSRRSGRAWYLGLDKEGRVMKGNRVKKTKAAAHFVPKLLEVAMYREPSLHSVPETSPSSPPAH

SEQ ID NO: 46 - protein, 225 aa, Rattus norvegicus
MAALASSLIRQKREVREPGGSRPVSAQRRVCPRGTKSLCQKQLLILLSKVRLCGGRPTRQDRGPEPQLKGIVTKLFCRQGFYLQANPDGSIQGTPEDTSSFTHFNLIPVGLRVVTIQSAKLGHYMAMNAEGLLYSSPHFTAECRFKECVFENYYVLYASALYRQRRSGRAWYLGLDKEGRVMKGNRVKKTKAAAHFVPKLLEVAVYREPSLHSVPETSPSSPPAH

SEQ ID NO: 47 - protein, 225 aa, Bos taurus
MAALASSLIRQKREVREPGGSRPVSAQRRVCPRGTKSLCQKQLLILLSKVRLCGGRPARTDRGPEPQLKGIVTKLFCRQGFYLQANPDGSIQGTPEDTSSFTHFNLIPVGLRVVTIQSAKLGHYMAMNAEGLLYSSPHFTAECRFKECVFENYYVLYASALYRQRRSGRAWYLGLDKEGRVMKGNRVKKTKAAAHFVPKLLEVAMYREPSLHSVPETSPSSPPAP

SEQ ID NO: 48 - protein, 181 aa, Homo sapiens
MESKEPQLKGIVTRLFSQQGYFLQMHPDGTIDGTKDENSDYTLFNLIPVGLRVVAIQGVKASLYVAMNGEGYLYSSDVFTPECKFKESVFENYYVIYSSTLYRQQESGRAWFLGLNKEGQIMKGNRVKKTKPSSHFVPKPIEVCMYREQSLHEIGEKQGRSRKSSGTPTMNGGKVVNQDST

SEQ ID NO: 49 - protein, 181 aa, Mus musculus
MESKEPQLKGIVTRLFSQQGYFLQMHPDGTIDGTKDENSDYTLFNLIPVGLRVVAIQGVKASLYVAMNGEGYLYSSDVFTPECKFKESVFENYYVIYSSTLYRQQESGRAWFLGLNKEGQIMKGNRVKKTKPSSHFVPKPIEVCMYREPSLHEIGEKQGRSRKSSGTPTMNGGKVVNQDST

SEQ ID NO: 50 - protein, 243 aa, Rattus norvegicus
  MOD_RES (225)..(225): Any amino acid
MAAAIASSLIRQKRQARESNSDRVSASKRRSSPSKDGRSLCERHVLGVFSKVRFCSGRKRPVRRRPEPQLKGIVTRLFSQQGYFLQMHPDGTIDGTKDENSDYTLFNLIPVGLRVVAIQGVKASLYVAMNGEGYLYSSDVFTPECKFKESVFENYYVIYSSTLYRQQESGRAWFLGLNKEGQIMKGNRVKKTKPSSHFVPKPIEVCMYREPSLHEIGEKQGRSRXSSGTPTMNGGKVVNQDST

SEQ ID NO: 51 - protein, 181 aa, Bos taurus; identical to SEQ ID NO: 49

SEQ ID NO: 52 - protein, 245 aa, Homo sapiens
MAAAIASSLIRQKRQAREREKSNACKCVSSPSKGKTSCDKNKLNVFSRVKLFGSKKRRRRRPEPQLKGIVTKLYSRQGYHLQLQADGTIDGTKDEDSTYTLFNLIPVGLRVVAIQGVQTKLYLAMNSEGYLYTSELFTPECKFKESVFENYYVTYSSMIYRQQQSGRGWYLGLNKEGEIMKGNHVKKNKPAAHFLPKPLKVAMYKEPSLHDLTEFSRSGSGTPTKSRSVSGVLNGGKSMSHNEST

SEQ ID NO: 53 - protein, 245 aa, Mus musculus
MAAAIASSLIRQKRQAREREKSNACKCVSSPSKGKTSCDKNKLNVFSRVKLFGSKKRRRRRPEPQLKGIVTKLYSRQGYHLQLQADGTIDGTKDEDSTYTLFNLIPVGLRVVAIQGVQTKLYLAMNSEGYLYTSEHFTPECKFKESVFENYYVTYSSMIYRQQQSGRGWYLGLNKEGEIMKGNHVKKNKPAAHFLPKPLKVAMYKEPSLHDLTEFSRSGSGTPTKSRSVSGVLNGGKSMSHNEST

SEQ ID NO: 54 - protein, 245 aa, Rattus norvegicus; identical to SEQ ID NO: 53

SEQ ID NO: 55 - protein, 245 aa, Bos taurus; identical to SEQ ID NO: 53

SEQ ID NO: 56 - protein, 252 aa, Homo sapiens
MVKPVPLFRRTDFKLLLCNHKDLFFLRVSKLLDCFSPKSMWFLWNIFSKGTHMLQCLCGKSLKKNKNPTDPQLKGIVTRLYCRQGYYLQMHPDGALDGTKDDSTNSTLFNLIPVGLRVVAIQGVKTGLYIAMNGEGYLYPSELFTPECKFKESVFENYYVIYSSMLYRQQESGRAWFLGLNKEGQAMKGNRVKKTKPAAHFLPKPLEVAMYREPSLHDVGETVPKPGVTPSKSTSASAIMNGGKPVNKSKTT

SEQ ID NO: 57 - protein, 247 aa, Mus musculus
MAAAIASGLIRQKRQAREQHWDRPSASRRRSSPSKNRGLCNGNLVDIFSKVRIFGLKKRRLRRQDPQLKGIVTRLYCRQGYYLQMHPDGALDGTKDDSTNSTLFNLIPVGLRVVAIQGVKTGLYIAMNGEGYLYPSELFTPECKFKESVFENYYVIYSSMLYRQQESGRAWFLGLNKEGQVMKGNRVKKTKPAAHFLPKPLEVAMYREPSLHDVGETVPKAGVTPSKSTSASAIMNGGKPVNKCKTT

SEQ ID NO: 58 - protein, 247 aa, Rattus norvegicus; identical to SEQ ID NO: 57

SEQ ID NO: 59 - protein, 252 aa, Bos taurus
MVKPVPLFRRTDFKLLLCNHKDLFFLRVSKLLDCFSPKSMWFLWNIFSKGTHMLQCLCGKSLKKNKNPTDPQLKGIVTRLYCRQGYYLQMHPDGALDGTKEDSTNSTLFNLIPVGLRVVAIQGVKTGLYVAMNGEGYLYPSELFTPECKFKESVFENYYVIYSSMLYRQQESGRAWFLGLNKEGQVMKGNRVKKTKPAAHFLPKPLEVAMYREPSLHDVGETVPKAGVTPSKSTSASAIMNGGKPVNKSKTT

SEQ ID NO: 60 - protein, 218 aa, Mus musculus
MARKWNGRAVARALVLATLWLAVSGRPLAQQSQSVSDEDPLFLYGWGKITRLQYLYSAGPYVSNCFLRIRSDGSVDCEEDQNERNLLEFRAVALKTIAIKDVSSVRYLCMSADGKIYGLIRYSEEDCTFREEMDCLGYNQYRSMKHHLHIIFIQAKPREQLQDQKPSNFIPVFHRSFFETGDQLRSKMFSLPLESDSMDPFRMVEDVDHLVKSPSFQK

SEQ ID NO: 61 - protein, 218 aa, Rattus norvegicus
MARKWSGRIVARALVLATLWLAVSGRPLVQQSQSVSDEGPLFLYGWGKITRLQYLYSAGPYVSNCFLRIRSDGSVDCEEDQNERNLLEFRAVALKTIAIKDVSSVRYLCMSADGKIYGLIRYSEEDCTFREEMDCLGYNQYRSMKHHLHIIFIKAKPREQLQGQKPSNFIPIFHRSFFESTDQLRSKMFSLPLESDSMDPFRMVEDVDHLVKSPSFQK

SEQ ID NO: 62 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 63 - protein, 353 aa, Bos taurus
MNATEDISESSSALRSVITVRCSPVPARRAPRELHAQPLEKLSGTQGQHRRRKTQQKQRSLPALRALERTAAGRARPIPGLKRHLALARATLLFLREPRSRLAPSRGTKASGPPPSLPHPHRQICAQSSEPEGGAMRSAPSRCAVARALVLAGLWLAAAGRPLAFSDAGPHVHYGWGESVRLRHLYTAGPQGLYSCFLRIHSDGAVDCAQVQSAHSLMEIRAVALSTVAIKGERSVLYLCMDADGKMQGLTQYSAEDCAFEEEIRPDGYNVYWSRKHHLPVSLSSSRQRQLFKSRGFLPLSHFLPMLSTIPAEPEDLQEPLKPDFFLPLKTDSMDPFGLATKLGSVKSPSFYN

SEQ ID NO: 64 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 65 - protein, 212 aa, Mus musculus
MAPLTEVGAFLGGLEGLGQQVGSHFLLPPAGERPPLLGERRGALERGARGGPGSVELAHLHGILRRRQLYCRTGFHLQILPDGTVQGTRQDHSLFGILEFISVAVGLVSIRGVDSGLYLGMNDKGELYGSEKLTSECIFREQFEENWYNTYSSNIYKHGNTGRRYFVALNKDGTPRDGARSKRRQKFTHFLPRPVDPERVPELYKDLLMYTG

SEQ ID NO: 66 - protein, 212 aa, Rattus norvegicus
MAPLTEVGAFLGGLEGLGQQVGSHFLLPPAGERPPLLGERRGALERGARGGPGSVELAHLHGILRRRQLYCRTGFHLQILPDGSVQGTRQDHSLFGILEFISVAVGLVSIRGVDSGLYLGMNGKGELYGSEKLTSECIFREQFEENWYNTYSSNIYKHGDTGRRYFVALNKDGTPRDGARSKRHQKFTHFLPRPVDPERVPELYKDLLVYTG

SEQ ID NO: 67 - protein, 211 aa, Bos taurus
MAPLAEVGGFLGGLEGLGQQVGSHFLLPPAGERPPLLGERRSAAERGARGGPGAAELAHLHGFLRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYS

SEQ ID NO: 68 - protein, 208 aa, Homo sapiens
MDSDETGFEHSGLWVSVLAGLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 69 - protein, 208 aa, Rattus norvegicus
MDWMKSRVGAPGLWVCLLLPVFLLGVCEAYPISDSSPLLQFGGQVRQRYLYTDDDQDTEAHLEIREDGTVVGTAHRSPESLLELKALKPGVIQILGVKASRFLCQQPDGTLYGSPHFDPEACSFRELLLKDGYNVYQSEAHGLPLRLPQKDSQDPATRGPVRFLPMPGLPHEPQEQPGVLPPEPPDVGSSDPLSMVEPLQGRSPSYAS

SEQ ID NO: 70 - protein, 209 aa, Bos taurus
MGWDEAKFKHLGLWVPVLAVLLLGTCRAHPIPDSSPLLQFGGQVRQRYLYTDDAQETEAHLEIRADGTVVGAARQSPESLLELKALKPGVIQILGVKTSRFLCQGPDGKLYGSLHFDPKACSFRELLLEDGYNVYQSETLGLPLRLPPQRSSNRDPAPRGPARFLPLPGLPAAPPDPPGILAPEPPDVGSSDPLSMVGPSYGRSPSYTS

SEQ ID NO: 71 - protein, 170 aa, Homo sapiens
MRRRLWLGLAWLLLARAPDAAGTPSASRGPRSYPHLEGDVRWRRLFSSTHFFLRVDPGGRVQGTRWRHGQDSILEIRSVHVGVVVIKAVSSGFYVAMNRRGRLYGSRLYTVDCRFRERIEENGHNTYASQRWRRRGQPMFLALDRRGGPRPGGRTRRYHLSAHFLPVLVS

SEQ ID NO: 72 - protein, 126 aa, Mus musculus
MRSRLWLGLAWLLLARAPGAPGGYPHLEGDVRWRRLFSSTHFFLRVDLGGRVQGTRWRHGQDSIVEIRSVRVGTVVIKAVYSGFYVAMNRRGRLYGSVPGAHRGERLQHIRLATLEAPRPTHVPGT

SEQ ID NO: 73 - protein, 162 aa, Rattus norvegicus
MRRRLWLGLAWLLLARAPGAPGGYPHLEGDVRWRRLFSSTHFFLRVDPGGRVQGTRWRHGQDSIVEIRSVRVGTVVIKAVYSGFYVAMNRRGRLYGSRVYSVDCRFRERIEENGYNTYASRRWRHHGRPMFLALDSQGIPRQGRRTRRHQLSTHFLPVLVSS

SEQ ID NO: 74 - protein, 170 aa, Bos taurus
MRGRLWLGLVWLLLARAPGTAGTLNTPRRPRSYPHLEGDVRWRRLFSSTHFFLLVDPSGRVQGTRWRDNPDSVLEIRSIRVGVVVLKAVHSGFYVAMNRLGRLYGSRFCAAHCRFRERIEENGYNTYASVRWRHQGRPMFLALDGRGAPRLGGRTQRHHPSTLFLPVLVS

SEQ ID NO: 75 - protein, 251 aa, Homo sapiens
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 76 - protein, 251 aa, Mus musculus
MLGTCLRLLVGVLCTVCSLGTARAYPDTSPLLGSNWGSLTHLYTATARTSYHLQIHRDGHVDGTPHQTIYSALMITSEDAGSVVITGAMTRRFLCMDLHGNIFGSLHFSPENCKFRQWTLENGYDVYLSQKHHYLVSLGRAKRIFQPGTNPPPFSQFLARRNEVPLLHFYTVRPRRHTRSAEDPPERDPLNVLKPRPRATPVPVSCSRELPSAEEGGPAASDPLGVLRRGRGDARGGAGGADRCRPFPRFV

SEQ ID NO: 77 - protein, 251 aa, Rattus norvegicus
MLGACLRLLVGALCTVCSLGTARAYSDTSPLLGSNWGSLTHLYTATARNSYHLQIHRDGHVDGTPHQTIYSALMITSEDAGSVVIIGAMTRRFLCMDLRGNIFGSYHFSPENCRFRQWTLENGYDVYLSPKHHYLVSLGRSKRIFQPGTNPPPFSQFLARRNEVPLLHFYTARPRRHTRSAEDPPERDPLNVLKPRPRATPIPVSCSRELPSAEEGGPAASDPLGVLRRGRGDARRGAGGTDRCRPFPRFV

SEQ ID NO: 78 - protein, 245 aa, Bos taurus
MLGARLGLWVCTLSCVVQAYPNSSPLLGSSWGGLTHLYTATARNSYHLQIHGDGHVDGSPQQTVYSALMIRSEDAGFVVITGVMSRRYLCMDFTGNIFGSHHFSPESCRFRQRTLENGYDVYHSPQHRFLVSLGRAKRAFLPGTNPPPYAQFLSRRNEIPLPHFAATARPRRHTRSAHDSGDPLSVLKPRARATPVPAACSQELPSAEDSGPAASDPLGVLRGHRLDVRAGSAGAERCRPFPGFA

SEQ ID NO: 79 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 80 - protein, 820 aa, Mus musculus
MWGWKCLLFWAVLVTATLCTARPAPTLPEQAQPWGVPVEVESLLVHPGDLLQLRCRLRDDVQSINWLRDGVQLVESNRTRITGEEVEVRDSIPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMLGSVIIYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSIPLDQYSPSFPDTRSSTCSSGEDSVFSHEPLPEEPCLPRHPTQLANSGLKRR

SEQ ID NO: 81 - protein, 822 aa, Rattus norvegicus
MWGWRGLLFWAVLVTATLCTARPAPTLPEQAQPWGVPVEVESLLVHPGDLLQLRCRLRDDVQSINWLRDGVQLAESNRTRITGEEVEVRDSIPADSGLYACVTNSPSGSDTTYFSVNVSDALPSSEDDDDDDDSSSEEKETDNTKPNRRPVAPYWTSPEKMEKKLHAVPAAKTVKFKCPSSGTPSPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYDQILKTAGVNTTDKEMEVLHLRNVSFEDAGEYTCLAGNSIGLSHHSAWLTVLEALEERPAVMTSPLYLEIIIYCTGAFLISCMVGSVIIYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPNPGVPVEELFKLLKEGHRMDKPSNCTNELYMMMRDCWNAVPSQRPTFKQLVEDLDRIVALTSNQEYLDLSMPLDQDSPSFPDTRSSTCSSGEDSVFSHEPFPEEPCLPRHPTQLANGGLNRR

SEQ ID NO: 82 - protein, 820 aa, Bos taurus
MWSRKCLLFWAVLVTATLCTAKPAPTLPEQAQPWGAPVEVESLLVHPGDLLQLRCRLRDDVQSINWLRDGVQLADSNRTRITGEEVEVRGSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIIYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDRPNRVTKVAVKMLKSDATEKDLSDLISEMEMMKMIGKHKNIINLLGACTQDGPLYVIVEYASKGNLREYLQARRPPGLEYCYNPSHHPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 83 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 84 - protein, 821 aa, Mus musculus
MVSWGRFICLVLVTMATLSLARPSFSLVEDTTLEPEEPPTKYQISQPEAYVVAPGESLELQCMLKDAAVISWTKDGVHLGPNNRTVLIGEYLQIKGATPRDSGLYACTAARTVDSETWIFMVNVTDAISSGDDEDDTDSSEDVVSENRSNQRAPYWTNTEKMEKRLHACPAANTVKFRCPAGGNPTSTMRWLKNGKEFKQEHRIGGYKVRNQHWSLIMESVVPSDKGNYTCLVENEYGSINHTYHLDVVERSPHRPILQAGLPANASTVVGGDVEFVCKVYSDAQPHIQWIKHVEKNGSKNGPDGLPYLKVLKAAGVNTTDKEIEVLYIRNVTFEDAGEYTCLAGNSIGISFHSAWLTVLPAPVREKEITASPDYLEIAIYCIGVFLIACMVVTVIFCRMKTTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPTNCTNELYMMMRDCWHAVPSQRPTFKQLVEDLDRILTLTTNEEYLDLTQPLEQYSPSYPDTRSSCSSGDDSVFSPDPMPYEPCLPQYPHINGSVKT

SEQ ID NO: 85 - protein, 841 aa, Rattus norvegicus
MGLPSTWRYGTGPGIGTVTMVSWGRFICLVLVTMATLSLARPSFSLVEDTTLEPEEPPTKYQISQPEACVVAPGESLELRCMLKDAAVISWTKDGVHLGPNNRTVLIGEYLQIKGATPRDSGLYACAAARTVDSETLYFMVNVTDAISSGDDEDDTDSSEDFVSENRSNQRAPYWTNTEKMEKRLHAVPAANTVKFRCPAGGNPTPTMRWLKNGKEFKQEHRIGGYKVRNQHWSLIMESVVPSDKGNYTCLVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEMDAGEYICKVSNYIGQANQSAWLTVLPKQQAPVREKEITASPDYLEIAIYCIGVFLIACMVVTVIFCRMKTTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDRPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPTNCTNELYMMMRDCWHAVPSQRPTFKQLVEDLDRILTLTTNEEYLDLTQPLEQYSPSYPDTRSSCSSGDDSVFSPDPMPYDPCLPQYPHINGSVKT

SEQ ID NO: 86 - protein, 839 aa, Bos taurus
MGLTSTWRYGRGQGIGTVTMVSWGRFLCLVVVTMATLSLARPSFNLVDDTTVEPEEPPTKYQISQPEVYVAAPRESLELRCLLRDAAMISWTKDGVHLGPNNRTVLIGEYLQIKGATPRDSGLYACTAARNVDSETVYFMVNVTDAISSGDDEDDADGSEDFVSENSNSKRAPYWTNTEKMEKRLHAVPAANTVKFRCPAGGNPTPTMRWLKNGKEFKQEHRIGGYKVRNQHWSLIMESVVPSDKGNYTCVVENDYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPVREKEIPASPDYLEIAIYCIGVFFIACMVVTVILCRMRNTTKKPDFSSQPAVHKLTKRIPLRRQVSAESSSSMNSNTPLVRITTRLSSTADTPMLAGVSEYELPEDPKWEFPRDKLTLGKPLGEGCFGQVVMAEAVGIDKEKPKEAVTVAVKMLKDDATEKDLSDLVSEMEMMKMIGKHKNIINLLGACTQDGPLYVIVEYASKGNLREYLRARRPPGMEYSYDINRVPEEQMAFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQLLEQYSPSYPDTRSSCSSGDDSVFSPDPMPYEPCLPQYPHRNGSVKT

SEQ ID NO: 87 - protein, 806 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 88 - protein, 800 aa, Mus musculus
MVVPACVLVFCVAVVAGATSEPPGPEQRVVRRAAEVPGPEPSQQEQVAFGSGDTVELSCHPPGGAPTGPTVWAKDGTGLVASHRILVGPQRLQVLNASHEDAGVYSCQHRLTRRVLCHFSVRVTDAPSSGDDEDGEDVAEDTGAPYWTRPERMDKKLLAVPAANTVRFRCPAAGNPTPSISWLKNGKEFRGEHRIGGIKLRHQQWSLVMESVVPSDRGNYTCVVENKFGSIRQTYTLDVLERSPHRPILQAGLPANQTAILGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELMETDEAGSVYAGVLSYGVVFFLFILVVAAVILCRLRSPPKKGLGSPTVHKVSRFPLKRQVSLESNSSMNSNTPLVRIARLSSGEGPVLANVSELELPADPKWELSRTRLTLGKPLGEGCFGQVVMAEAIGIDKDRTAKPVTVAVKMLKDDATDKDLSDLVSEMEMMKMIGKHKNIINLLGACTQGGPLYVLVEYAAKGNLREFLRARRPPGMDYSFDACRLPEEQLTCKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPASCTHDLYMIMRECWHAVPSQRPTFKQLVEDLDRILTVTSTDEYLDLSVPFEQYSPGGQDTPSSSSSGDDSVFTHDLLPPGPPSNGGPRT

SEQ ID NO: 89 - protein, 800 aa, Rattus norvegicus
MVVPACVLVFCVAVVAGVTSEPPGPEQRVGRRAAEVPGPEPSQQEQVAFGSGDTVELSCHPPGGAPTGPTLWAKDGVGLVASHRILVGPQRLQVLNATHEDAGVYSCQQRLTRRVLCHFSVRVTDAPSSGDDEDGEDVAEDTGAPYWTRPERMDKKLLAVPAANTVRFRCPAAGNPTPSIPWLKNGKEFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDRELEVLSLHNVTFEDAGEYTCLAGNSIGFSHHSAWLVVLPAEEELMEVDEAGSVYAGVLSYGVGFFLFILVVAAVTLCRLRSPPKKGLGSPTVHKVSRFPLKRQVSLESNSSMNSNTPLVRIARLSSGEGPVLANVSELELPADPKWELSRTRLTLGKPLGEGCFGQVVMAEAIGIDKDRTAKPVTVAVKMLKDDATDKDLSDLVSEMEMMKMIGKHKNIINLLGACTQGGPLYVLVEYAAKGNLREFLRARRPPGMDYSFDACRLPEEQLTCKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAVPSQRPTFKQLVEDLDRILTVTSTDEYLDLSVPFEQYSPGGQDTPSSSSSGDDSVFTHDLLPPGPPSNGGPRT

SEQ ID NO: 90 - protein, 802 aa, Bos taurus
MGAPARALAFCVAVAVMTGAALGSPGVEPRVARRAAEVPGPEPSPQERAFGSGDTVELSCRLPAGVPTEPTVWVKDGVGLAPSDRVLVGPQRLQVLNASHEDAGAYSCRQRLSQRLLCLFSVRVTDAPSSGDDEGGDDEAEDTAGAPYWTRPERMDKKLLAVPAANTVRFRCPAAGNPTPSITWLKNGKEFRGEHRIGGIKLRQQQWSLVMESVVPSDRGNYTCVVENKFGRIQQTYTLDVLERSPHRPILQAGLPANQTAVLGSDVEFHCKVYSDAQPHIQWLKHVEVNGSKVGPDGTPYVTVLKTAGANTTDKELEVLSLRNVTFEDAGEYTCLAGNSIGFSHHSAWLVVLPAEEELVEAGEAGGVFAGVLSYGLGFLLFILAVAAVTLYRLRSPPKKGLGSPAVHKVSRFPLKRQVSLESSSSMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREYLRARRPPGTDYSFDTCRLPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSVPFEQYSPGGQDTPSSGSSGDDSVFAHDLLPPAPSGSGGSRT

SEQ ID NO: 91 - protein, 802 aa, Homo sapiens
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTDIILYASGSLALAVLLLLAGLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 92 - protein, 799 aa, Mus musculus
MWLLLALLSIFQGTPALSLEASEEMEQEPCLAPILEQQEQVLTVALGQPVRLCCGRTERGRHWYKEGSRLASAGRVRGWRGRLEIASFLPEDAGRYLCLARGSMTVVHNLTLLMDDSLTSISNDEDPKTLSSSSSGHVYPQQAPYWTHPQRMEKKLHAVPAGNTVKFRCPAAGNPMPTIHWLKDGQAFHGENRIGGIRLRHQHWSLVMESVVPSDRGTYTCLVENSLGSIRYSYLLDVLERSPHRPILQAGLPANTTAVVGSDVELLCKVYSDAQPHIQWLKHVVINGSSFGADGFPYVQVLKTTDINSSEVEVLYLRNVSAEDAGEYTCLAGNSIGLSYQSAWLTVLPEEDLTWTTATPEARYTDIILYVSGSLVLLVLLLLAGVYHRQVIRGHYSRQPVTIQKLSRFPLARQFSLESRSSGKSSLSLVRGVRLSSSGPPLLTGLVNLDLPLDPLWEFPRDRLVLGKPLGEGCFGQVVRAEAFGMDPSRPDQTSTVAVKMLKDNASDKDLADLVSEMEVMKLIGRHKNIINLLGVCTQEGPLYVIVECAAKGNLREFLRARRPPGPDLSPDGPRSSEGPLSFPALVSCAYQVARGMQYLESRKCIHRDLAARNVLVTEDDVMKIADFGLARGVHHIDYYKKTSNGRLPVKWMAPEALFDRVYTHQSDVWSFGILLWEIFTLGGSPYPGIPVEELFSLLREGHRMERPPNCPSELYGLMRECWHAVPSQRPTFKQLVEALDKVLLAVSEEYLDLRLTFGPFSPSNGDASSTCSSSDSVFSHDPLPLEPSPFPFSDSQTT

SEQ ID NO: 93 - protein, 800 aa, Rattus norvegicus
MWLLLALLSIFQETPAFSLEASEEMEQEPCPAPISEQQEQVLTVALGQPVRLCCGRTERGRHWYKEGSRLASAGRVRGWRGRLEIASFLPEDAGRYLCLARGSMTVVHNLTLIMDDSLPSINNEDPKTLSSSSSGHSYLQQAPYWTHPQRMEKKLHAVPAGNTVKFRCPAAGNPMPTIHWLKNGQAFHGENRIGGIRLRHQHWSLVMESVVPSDRGTYTCLVENSLGSIRYSYLLDVLERSPHRPILQAGLPANTTAVVGSNVELLCKVYSDAQPHIQWLKHIVINGSSFGADGFPYVQVLKTTDINSSEVEVLYLRNVSAEDAGEYTCLAGNSIGLSYQSAWLTVLPAEEEDLAWTTATSEARYTDIILYVSGSLALVLLLLLAGVYHRQAIHGHHSRQPVTVQKLSRFPLARQFSLESRSSGKSSLSLVRGVRLSSSGPPLLTGLVSLDLPLDPLWEFPRDRLVLGKPLGEGCFGQVVRAEALGMDSSRPDQTSTVAVKMLKDNASDKDLADLISEMEMMKLIGRHKNIINLLGVCTQEGPLYVIVEYAAKGNLREFLRARRPPGPDLSPDGPRSSEGPLSFPALVSCAYQVARGMQYLESRKCIHRDLAARNVLVTEDDVMKIADFGLARGVHHIDYYKKTSNGRLPVKWMAPEALFDRVYTHQSDVWSFGILLWEIFTLGGSPYPGIPVEELFSLLREGHRMERPPNCPSELYGLMRECWHAAPSQRPTFKQLVEALDKVLLAVSEEYLDLRLTFGPYSPNNGDASSTCSSSDSVFSHDPLPLEPSPFPFPEAQTT

SEQ ID NO: 94 - protein, 800 aa, Bos taurus
MRLLLVLLGVLLGAPGAPALSFEASEETELEPCLAPSPEQQEQELTVALGQPVRLCCGRAERSGHWYKEGSRLTPAGRVRGWRGRLEIASFLPEDAGQYLCLSRGSLLLHNVTLVVDDSMTSSNGDEDPKIHRGPLNGHVYPQQAPYWTHPQRMEKKLHAVPAGNTVKFRCPAAGNPMPTIRWLKDGQDFHGEHRIGGIRLRHQHWSLVMESVVPSDRGTYTCLVENSLGSIRYSYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDLTWTATAPEGRYTDIILYSSGSLALIVFLLLVGLYRRQTLLTRHHRQPATVQKLSRFPLARQFSLESGSSAKSSLSLVRGVRLSSSGPPLLAGLVSLDLPLDPLWEFPRDRLVLGKPLGEGCFGQVVCAEAFGMDPTRPDQASTVAVKMLKDNASDKDLADLVSEMEVMKLIGRHKNIINLLGVCTQEGPLYVIVECAAKGNLREFLRARRPPGPDLSPDGPRSSEGPLSFPALVSCAYQVARGMQYLESRKCIHRDLAARNVLVTEDNVMKIADFGLARGIHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPAGGDASSTCSSSDSVFSHDPLPLRPSSFSFPGVQT

SEQ ID NO: 95 - DNA, 18 bp, Artificial Sequence
  Description of Artificial Sequence Synthetic primer
caagctgcacccgagctt

SEQ ID NO: 96 - DNA, 22 bp, Artificial Sequence
  Description of Artificial Sequence Synthetic primer
tgattcgatgaaggtgatttcg

SEQ ID NO: 97 - DNA, 19 bp, Artificial Sequence
  Description of Artificial Sequence Synthetic primer
tgatgattggacgctgcaa

SEQ ID NO: 98 - DNA, 18 bp, Artificial Sequence
  Description of Artificial Sequence Synthetic primer
cattggccacatcgcttg

SEQ ID NO: 99 - DNA, 19 bp, Artificial Sequence
  Description of Artificial Sequence Synthetic primer
accaaggccaaaacgacag

SEQ ID NO: 100 - DNA, 22 bp, Artificial Sequence
  Description of Artificial Sequence Synthetic primer
gggctcacattgttctacttga

SEQ ID NO: 101 - DNA, 20 bp, Artificial Sequence
  Description of Artificial Sequence Synthetic primer
ggcggagcatatgctgatcc

SEQ ID NO: 102 - DNA, 20 bp, Artificial Sequence
  Description of Artificial Sequence Synthetic primer
ccacaggcactagggaaggc

SEQ ID NO: 103 - DNA, 23 bp, Artificial Sequence
  Description of Artificial Sequence Synthetic primer
tcaacctcgtcttcaagtggatt

SEQ ID NO: 104 - DNA, 24 bp, Artificial Sequence
  Description of Artificial Sequence Synthetic primer
ctgctttattataggcacggagct

SEQ ID NO: 105 - DNA, 21 bp, Artificial Sequence
  Description of Artificial Sequence Synthetic primer
actgccacacctccagtcatt

SEQ ID NO: 106 - DNA, 21 bp, Artificial Sequence
  Description of Artificial Sequence Synthetic primer
ctttgcctcactcaggattgg

SEQ ID NO: 107 - DNA, 20 bp, Artificial Sequence
  Description of Artificial Sequence Synthetic primer
cggacaggattgacagattg

SEQ ID NO: 108 - DNA, 20 bp, Artificial Sequence
  Description of Artificial Sequence Synthetic primer
caaatcgctccaccaactaa